(19) United States
(12) Patent Application Publication
(10) Pub. No.: US 2017/0304815 A1
(43) Pub. Date: Oct. 26, 2017

(54) Antimicrobial And Biological Active Polymer Composites And Related Methods, Materials and Devices

(71) Applicants: David John VACHON, Spokane, WA (US); Scott Frithjof NORDAL, Spokane, WA (US); Dana Christopher WALTERS, Spokane, WA (US); IASIS MOLECULAR SCIENCES, Spokane, WA (US)

(72) Inventor: David J. VACHON, Spokane, WA (US)

(21) Appl. No.: 15/509,834

(22) PCT Filed: Sep. 9, 2015
(86) PCT No.: PCT/US15/49255

§ 371 Date: Mar. 8, 2017

Related U.S. Application Data
(60) Provisional application No. 62/047,655, filed on Sep. 9, 2014.

(51) Int. Cl. B01J 45/00; A01N 25/10; A01N 25/16; A01N 33/12; A01N 41/04; A01N 41/12; A01N 43/40; A01N 59/00; A01N 59/16; A61H 19/00; A61K 31/14; A61K 31/155; A61K 31/616; A61K 47/58; G01N 33/18; C09J 183/04; C09J 11/08; C09J 9/00; C09D 201/00; C09D 5/16; C09D 5/14; C08L 83/04; C08L 75/04; C08L 63/00; C08L 57/06; C08L 23/12; C08J 9/36; C08J 3/12; B65D 65/42; B65D 65/40; B01J 39/20; A61L 29/16; A61L 29/08; A61L 27/54; A61L 27/18; A61L 26/00; A61L 15/58; A61L 15/44; A61L 15/24; A61K 33/38; G01N 33/24

(52) U.S. Cl. CPC B01J 45/00; G01N 33/24; A01N 25/10; A01N 25/16; A01N 33/12; A01N 41/04; A01N 41/12; A01N 43/40; A01N 59/00; A01N 59/16; A61H 19/44; A61K 31/14; A61K 31/155; A61K 31/616; A61K 47/585; G01N 33/18; C09J 183/04; C09J 11/08; C09J 9/00; C09D 201/00; C09D 5/1637; C09D 5/14; C08L 83/04; C08L 75/04; C08L 63/00; C08L 57/06; C08L 23/12; C08J 9/365; C08J 3/12; B65D 65/42; B65D 65/40; B01J 39/20; A61L 29/16; A61L 29/085; A61L 27/54; A61L 27/18; A61L 26/0085; A61L 26/0066; A61L 15/58; A61L 15/44; A61L 15/24; A61K 33/38; A61L 2300/104; A61L 2300/11; A61L 2300/206; A61L 2300/208; A61L 2300/404; C08J 2205/05; G01N 2033/245; C08J 2325/08; C08J 2375/04; C08J 2425/02; C08J 2425/08; A61L 2300/606; C08J 2325/02

(57) ABSTRACT

Biologically activated ion-exchange polymer salts are made by exchanging biologically active ionic agents onto ion-exchange polymers. The activated polymers are uniquely surface active and stable to thermal degradation and chemical and other forms of decomposition. The activated ion-exchange polymer salts may be processed and combined with polymer precursors using novel methods and materials to produce stable, biologically activated polymer composites, including antimicrobial and antifouling polymer composites.

### TECHNICAL FIELD

The invention relates to biologically active materials, coatings and devices employing functionalized ion-exchange materials associated with active antimicrobial agents, therapeutic agents and other biologically active agents. In more detailed aspects the invention relates to polymer composites integrating biologically activated, functionalized ion-exchange materials.

### BACKGROUND

Increased human population and intermingling of populations have facilitated pathogen transmission and rendered it more difficult to control disease spread. Until 1987, the Centers for Disease Control and the American Hospital Association focused on patients as the principal vector for pathogen transmission, because the CDC regarded nosocomial infections to be generally unrelated to microbial contamination of (Cozad, A., and R. D. Jones. 2003) Evidence now clearly establishes that fomites (objects and surfaces that can become contaminated with pathogenic microorganisms) play a key role in spreading infection, including nosocomial infections. (Aitken, C., and D. J. Jeffries. 2001, Barker, J., D. Stevens, and S. F. Bloomfield. 2001)

Fomites readily serve as vehicles for transmission to living subjects (England, B. L. 1982; Ilaas, C. N., J. B. Rose, and C. P. Gerba. 1999; Reynolds, K. A., P. Watts. S. A. Boone, and C. P. Gerba. 2005; Sattar. S. A. 2001). Fomites readily become contaminated by direct contact with body secretions or fluids, soiled hands, aerosolized virus generated via talking, sneezing, coughing, or vomiting, or airborne virus settling after disturbance of a contaminated fomite. Once a fomite is contaminated, the transfer of contamination may readily occur between the contaminated fomite and another fomite or living host (Goldmann, D. A. 2000)

As rates of nosocomial and resistant infections in hospitals, dental offices, veterinary care centers, daycares, schools, gyms and other public places continue to increase, there is a growing urgency to develop biologically active materials that resist or prevent contamination of surfaces and materials present in these environments.

A related need exists for materials that disable or kill contaminating pathogens following contact with the materials, to prevent transmission from a fomite surface composed of, or treated with, the materials to another fomite or a living subject.

Additional objects that remain unsatisfied in the art include development of contamination-resistant and contamination-preventive and infection-preventive materials that can be utilized in a variety of materials, devices and applications.

Further beyond the reach of current technology are polymer materials having a broad range of intrinsic surface biologically active properties, where the materials can incorporate a large diversity of surface active agents and can be incorporated in diverse compositions and methods and adapted for broad use in clinical, hygiene, environmental, and therapeutic methods and materials.

### SUMMARY OF EXEMPLARY EMBODIMENTS OF THE INVENTION

The invention fulfills these needs and satisfies additional objects and advantages by providing novel polymer materials that are biologically activated by incorporating ionic biologically agents, for example ionic antimicrobial agents (e.g., ionic or ionizable forms of antibiotic agents, antiseptic agents, antifungal agents, etc.)

The incorporation of biologically active ionic agents in novel polymeric biomaterials and coatings of the invention is achieved by combining one or more ionic biologically active agents with an ion-exchange polymer salt, for example a functionalized ion-exchange resin material. In exemplary embodiments, a porous ion-exchange resin material is combined with a cationic or anionic biologically active agent (e.g., a cationic antibiotic or an oligodynamic metal) in an aqueous medium under conditions that mediate substitution of the ionic active agent onto the resin (by salt exchange)—typically by displacement of a similarly charged anionic or cationic counter-ion originally bound (ionically bound, electrostatically surface-associated, or adsorbed) onto the resin (or non-resin polymer) to form a substituted, biologically activated polymer salt.

In more detailed embodiments of the invention, a “biologically activated” ion-exchange polymer salt is constructed by ionically modifying the polymer salt to carry an ionic biologically active agent, for example, ionic silver (Ag⁺) substituted for a like-charged counter-ion originally bound on the resin, for example ionic sodium (Na⁺). The resulting activated ion-exchange polymer salt material is processed using novel materials and methods. In certain embodiments, larger, biologically activated particles of the polymer salt are processed using a novel size reducing milling technology to generate a fine particulate activated ion-exchange polymer salt resin product, milled to a high degree of particle size uniformity.

The biologically activated polymer salts of the invention are useful alone and in a diverse array of antimicrobial and other biologically active polymer “composite” materials. Within certain embodiments, a biologically activated fine particulate ion-exchange polymer salt material is combined with a thermoset or thermoplastic or photocuring polymer, or other curable polymer, or with water soluble polymers in order to form solid activated polymer composites.

In yet another detailed embodiment of the invention, an ion-exchange polymer salt is constructed by ionically modifying the polymer salt to carry an ionic agent, for example, ionic barium (Ba⁺⁺) substituted for a like-charged counter-ion originally bound on the resin, for example ionic sodium (Na⁺). These particulates can act as radiocontrast agents for X-ray imaging in methods and compositions currently employing barium sulfate, with less safety concerns (e.g., due to retention and/or soluble barium exposure). In illustrative embodiments radiocontrast effective ionic agents, exemplified by barium, are incorporated in an ion-exchange polymer salt, which can be used directly (although it will typically milled to a desired particle size) for gastrointestinal (GI) imaging, e.g., by delivering a suspension or colloid of the radioconstrast effective activated polymer salt to a GI tract of a patient (e.g., by ingestion) in conjunction with conventional barium GI imaging tools and methods. In related embodiments, catheters, endoscopes, laproscopic instruments and other devices are provided that integrate polymer composite materials made using radioconstrast effective activated polymer salts as described herein. In one exemplary embodiment, a portion (such as a longitudinal stripe) of an angioplasty tube is radiocontrast marked for localization within a vascular site by incorporating a radiocontrast effective activated polymer composite within a portion (e.g., a linear stripe portion) of an angioplasty tubing (e.g., by co-extrusion with another polymer).

Novel milling technologies are also provided herein employing a porous particulate ion-exchange polymer salt material activated by incorporation of an ionic biologically active agent. In exemplary embodiments, the activated porous polymer salt material is subjected to high energy milling employing a liquid non-solvent. The non-solvent liquid is added to the activated polymer salt particles before milling to occupy channels, voids and pores within the resin particles during milling. Occupancy of these channel and void spaces by the non-solvent surprisingly facilitates normalized particle rupture and size reduction to generate a fine particulate activated resin product in micro- and nano-meter particle diameter size ranges. These fine particles exhibit a high degree of size predictability and uniformity. These novel size properties of the activated polymer salt particles provide additional unexpected advantages, uses, biological activities and performance characteristics for these materials, particularly when combined with a thermoset or thermoplastic or photocuring polymer, or other curable polymer, to yield novel polymer composites that are curable to form solid materials, coatings, paints, laminates, and related materials, components and devices.

The methods, materials and composites of the invention can employ or integrate a large diversity of antimicrobial agents and activities. In additional embodiments, these methods, materials and composites can incorporate a host of other types of biologically active, ionic or ionizable agents, including a diverse array of clinically useful and therapeutic agents.

In certain embodiments of the invention, fine particulate biologically activated resin materials are incorporated in solid polymer composites, and these materials provide an astounding array of useful manufactures, textiles, objects, devices, coatings, laminates and the like for use in health care, institutional, environmental, laboratory and other settings. In exemplary applications, the materials and manufactures of the invention are useful in medical, dental, orthopedic and veterinary facilities, tools, materials, implants, devices and equipment.

In other exemplary embodiments the biologically activated resin materials are incorporated in solid polymer composites and the composites are pelletized for other applications including molding, extrusion, and other processing methods.

Other uses and constructions of the materials and methods herein are described for consumer products, textiles, apparel, athletic equipment and accessories, sports therapy and gymnasium facilities and equipment, lavatory and food service materials and equipment, transportation materials and equipment, and HVAC materials and equipment, among many other designs and applications.

Within certain embodiments of the invention, methods for producing fine particulate ion-exchange polymer salt materials are described, allowing for biological activation of the polymer salt by ionic association with a biologically active ionic agent. According to these methods generally, particles of a water-insoluble polysulfonated, polycarboxylated, polyaminated, or polyphosphorylated polymer salt material, for example a polymer salt of a cross-linked, functionalized resin, are combined with a biologically active ionic agent in an aqueous medium under conditions to allow substitution of the biologically active ionic agent by salt-exchange for a counter-ion (e.g., a sodium ion) initially associated with the ion-exchange polymer salt material. This yields a biologically activated ion-exchange polymer salt particle having the biologically active ionic agent ionically associated with the ion-exchange polymer salt material. According to the teachings herein, the biologically active ionic agent is thus rendered insoluble and will not freely dissociate from the biologically activated polymer salt material in deionized water.

A wide array of biologically useful ionic or ionizable drugs, compounds and other active agents can be employed to form the biologically activated ion-exchange polymer salts of the invention. In exemplary embodiments, the biologically active ionic agent is an anti-microbial agent. Suitable anti-microbial agents include ionic or ionizable antibiotics, antiseptics, antivirals, antiparasitics, and antifungals, and oligodynamic metals. In other exemplary embodiments, an oligodynamic metal selected from silver, copper, zinc, iron, gallium, or bismuth is employed. In other exemplary embodiments, a cationic antibiotic is employed. Exemplary cationic antibiotics include tetracyclines or anthracycline and aminoglycosides. In more detailed embodiments, a tetracycline is selected from tetracycline, doxycycline, minocycline, lymecycline, or apicycline, or combinations thereof and aminoglycosidcs include gentamicin and/or tobramycin. In yet additional exemplary embodiments, a cationic antiseptic is employed. Exemplary cationic antiseptics may comprise a guanidinium group (e.g., as exemplified by chlorhexidine or polyhcxamethylenebiguanide), or a quaternary ammonium group (e.g., as exemplified by chlorhexidine, benzalkonium, cetylpyridinium, cetrimonium (cetrimide) and quaternary ammonium).

In additional exemplary embodiments, anionic biologically active agents are incorporated by ionic association within the ion-exchange polymer salts of the invention. Exemplary biologically active anionic agents (including agents modifiable to an anionic form) include acetylsalicylic acid-CO₂—, dexamethasone sodium phosphate, fusidic acid (fusidate), and vitamin C (ascorbate), among others.

Exemplary ion-exchange polymer salts for use within the invention may comprise an ion-exchange polymer salt comprising one or more of a styrene, acrylic, acrylate, sulfonate, carboxylate, phosphate, protonated amine, ammonium, and/or quaternary ammonium functional group(s). In certain embodiments, the ion-exchange polymer salt material comprises a cross-linked polymer resin, for example a cross-linked styrene, acrylic, or acrylate polymer resin.

In more detailed aspects of the invention, novel biologically activated polymer “composites” and methods for preparing these composites, are provided. In exemplary embodiments, the activated composites are made by first providing an ion-exchange polymer salt, as summarized above. The ion-exchange polymer salt is typically a water-insoluble polysulfonated, polycarboxylated, polyaminated, or polyphosphorylated polymer salt. In exemplary embodiments the particles have a porous construction, with individual particles defining channel, void and pore space surrounded by walls and partitions of polymer salt material. The ion-exchange polymer salt particles are combined with a biologically active ionic agent in an aqueous medium to substitute the biologically active ionic agent by salt-exchange for a counter-ion initially associated with the ion-exchange polymer salt material. This yields a biologically activated porous ion-exchange polymer salt particle having the biologically active ionic agent ionically associated with the ion-exchange polymer salt material. By virtue of this novel preparation method and construction, the biologically active ionic agent is rendered insoluble, in that it will not freely dissociate from the insoluble ion-exchange polymer salt material in deionized water.

Following construction of the activated ion-exchange polymer salt material the material is dried to remove most or all of the water present in the original aqueous medium (e.g., water or an aqueous solution such as an alcohol). The biologically activated ion-exchange polymer salt particles are then milled by a high energy milling process. Generally this involves use of porous particles milled in the presence of a non-solvent liquid, which is added to occupy channel, void and pore spaces within the polymer salt particles. The non-solvent liquid provides compression resistance as described to oppose mechanical and pressure forces of milling as described, to mediate more efficient and uniform particle disruption and size reduction during milling.

The resultant fine particulate biologically activated ion-exchange polymer salt material is optionally blended with thermoset or thermoplastic or photocuring polymer precursors to form a fluid or semi-solid thermoset or thermoplastic or photocuring polymer (or other curable polymer) composite mixture. This mixture comprises the fine particles of biologically activated ion-exchange polymer salt thoroughly or incompletely admixed with the polymer precursors (e.g., to form a homogeneous or heterogeneous dispersions, or to blend the polymer salt particles only through a discrete portion of the composite mixture). After blending to a desired degree of mixing, the mixture of the fine particulate, activated polymer salt and curable polymer precursors may be hardened or “cured” to form a biologically activated solid polymer composite. Hardening or curing of the mixture may involve thermal-facilitated polymerization and/or cross-linking of the polymer precursors (e.g., attended by a heat-producing reaction, or facilitated by external heating). In other embodiments, hardening or curing of the composite mixture can involve polymerization or cross-linking of polymer precursors accompanying removal of water (e.g., normal drying at room temperature, optionally under vacuum) or removal of a non-aqueous, organic solvent (e.g., as in dry-curing of certain epoxy and lacquer composite mixtures of the invention). Alternatively, in the case of photocuring composite mixtures, polymerization and/or cross-linking of the polymer precursors to solidify or cure the mixture to a cured or substantially solid form may be mediated by external application of light energy (e.g., ultraviolet radiation from a photocuring device). Upon hardening or curing of the composite mixture, the fine particulate biologically activated polymer salt particles are integrated within the thermoset, thermoplastic, photocuring or other curable polymer matrix, collectively forming a solid biologically activated polymer composite.

Any biologically acceptable thermoset, thermoplastic, photocuring or other curable polymer can be employed within aspects of the invention relating to biological or biomedical materials and devices. For other embodiments that do not require the use of biologically acceptable polymers (e.g., for certain marine antifouling coatings), industrial grade materials are acceptable.

In exemplary embodiments, thermoset, thermoplastic, photocuring and other curable polymers for making polymer composites (by admixing with the fine particulate, biologically activated polymer salt), may be selected from, for example, a polysiloxane, polyalkylene, polyamide, epoxy, polycarbonate, polyester, vinyl, acrylic, polyurethane, plastisol (e.g., a suspension of polyvinylchloride or PVC), or polyvinylidinefluoride (PVDF) polymer, or mixtures thereof, while in other embodiments different polymers may be used (provided they are equivalent, i.e., operable within the methods and compositions of the invention, as described here). In certain embodiments, the polymer precursors comprise non-vulcanized silicone rubber precursors.

When silicone rubber polymer precursors are used, these can be combined so as to form a highly-adhesive silicone gel or liquid that is particularly useful in certain manufacturing methods and products of the invention. Silicone polymer composites can be cured under a range of conditions, for example at about 150 degrees for 5 to 10 minutes or with the addition of an appropriate photoactive catalyst, the polymer may be cured by exposure to UV radiation (e.g., using Momentive Performance Materials). Within more detailed embodiments, the biologically active ionic agent is an oligodynamic metal and the activated fine particulate product incorporating the oligodynamic metal is blended with silicone gel or liquid further comprising an oligodynamic metal darkens the hardened silicone product.

Also provided within the invention are materials and composites made according to the foregoing processes, and articles and devices incorporating these materials and composites. In certain aspects, biomaterials, products, tools and equipment are made that incorporate a fine particulate, biologically activated ion-exchange polymer salt or resin material as described. In more detailed aspects of the invention, biologically activated, stable polymer composites are provided that comprise a fine particulate polymer salt ionically associated with a biologically active ionic agent, where the polymer salt is dispersed within a thermoset or thermoplastic or photocuring polymer to form solid, biologically activated polymer composite. Biologically activated polymer composites of the invention remains intact and biologically active without substantial chemical degradation, oxidation, hydrolysis, chemical decomposition, or photodegradation of the integrated ionic biologically active agent (e.g., wherein the biologically active agent remains stable and retains most if not all of its biological activity during preparation of the ion-exchange polymer salt, and preparation and hardening/curing of the thermoset or thermoplastic or photocuring polymer).

Additional novel aspects of the invention include the provision of novel materials and methods for producing “self-regenerating” or “renewable” biomaterials and polymer composites. Polymer composites described herein can passively renew or regenerate their original surface biological activity, or can be rehabilitated, restored or recharged to approximate their initial (post-fabrication) biological activity, after being partially or completely chemically exhausted, reacted, degraded, decomposed or discharged. In exemplary embodiments, a fine particulate biologically activated ion-exchange resin material is integrated throughout a solid polymer structure to provide for passively renewable surface activation following discharge. For example, after a period of normal surface wear or erosion, polymer composites of the invention will often exhibit a measurable amount of “discharge” of the biologically active ionic agents, including by release or dissociation of activated ion-exchange resin material and/or biologically active ionic agents from the polymer surface, chemical reaction, decomposition, photodegradation, at the polymer surface, and or loss by erosion of micro- or nano-particles of the fine particulate ion-exchange polymer salt material embedded within the composite (or composite surface layer(s)). In these embodiments, “recharging” of biological activity at the polymer surface is passively mediated by erosive wear, which passively debrides an outermost layer of the composite and exposes underlying material that is fully charged with intact, non-discharged particles of the activated ion-exchange polymer salt bearing a full (original as fabricated) load of biologically active ionic agent—effectively restoring or replacing the original surface activity.

In related embodiments, erosive recharging is actively mediated, for example by debriding or polishing a partially discharged polymer composite surface with an abrasive paper, cloth, paste or solution. This manual resurfacing/recharging is likewise mediated by debriding a discharged surface layer of the polymer composite to expose fresh, non-discharged particles of the activated ion-exchange polymer salt bearing a full (comparable to original, e.g., at least 75%-90% of original surface activity) load of surface active biologically active ionic agent.

In alternative “recharging” constructions and methods, after partial discharge of biological activity from a polymer surface, the surface can be actively recharged by manual methods involving novel chemical re-treatment. For example, discharge of biologically active ionic agent comprising ionic silver (Ag⁺) from an activated polymer composite can occur when tissues or physiological fluids are contacted with the surface of the activated composite (e.g., by counter-ionic exchange of sodium (Na⁺) in the physiological fluid with silver ions originally “loaded” within the composite. This discharges some of the total silver ion activity (e.g., expressed in terms of antimicrobial activity) from an original “loading capacity” “selected sub-capacity loading” or “post-fabrication biological activity potential”. Under these circumstances, the invention provides novel materials and methods allowing for recharging or reloading (even above original loading or post-fabrication activity) of most or all of the original loading or activity potential, for example by treating a partially discharged composite surface with a solution of a silver salt (e.g., silver acetate or silver nitrate) to reload ionic silver in renewed ionic association with the ion-exchange polymer to regenerate the biologically activated polymer salt at the surface of the polymer composite. In other embodiments ionic antiseptics (e.g., benzalkonium-based antiseptics) can be similarly recharged as reloaded, biologically active ionic agents at a surface of a polymer composite (e.g., by wiping or saturating the surface with a benzalkonium chloride solution).

Additional novel materials and methods are provided where a polymer composite surface containing a biologically active ionic agent combined with a polymer salt integrated in the polymer composite can be newly “activated” by post-manufacturing surface treatment. Within these discrete methods and materials, the composite can be “surface activated” after manufacturing by chemically modifying the surface to a biologically activated condition. In one exemplary embodiment, a composite surface is activated by exposing the surface to peroxide (e.g., simply by wiping, immersing or spraying the surface with a peroxide solution). This generates superoxides at the surface of the material, rendering the surface strongly antimicrobial.

The forgoing and additional objects, features, aspects and advantages of the present invention will become apparent from the following detailed description.

### BRIEF DESCRIPTION OF THE DRAWINGS

Embodiments of the disclosure are described below with reference to the following accompanying drawings.

FIG. 1 is a graphical representation of results from a time to kill assay for Sulfonated polystyrene-co-divinylbenzene-Ag (2 wt %) (IRP69-Ag) modified silicone rubber (Q7-4750). Using an inoculum of 10⁸ CFU/mL of E. coli in synthetic urine (recipe), samples were incubated at 37° C. and at time points of 0, 3, 8, 16, 24, and 32 hours samples were removed from test and adherent bacteria removed and counts determined. The data reveal that after 3 hours a one-log reduction is observed and after 32 hours a 6-log reduction is evident. These data demonstrate surface properties and activities of the novel polymer composites of the invention provide surprising advantages of reducing the likelihood of bacterial colonization and survival (e.g., on an exterior surface and lumen of a urinary catheter).

### DETAILED DESCRIPTION OF EXEMPLARY EMBODIMENTS OF THE INVENTION

Described herein are compositions of polymeric ion-exchange materials incorporating biologically active ionic agents to form novel, biologically activated ion-exchange polymer salts. These materials are useful for a variety of purposes, including as stable biologically active constituents of uniquely functional solid polymer composites. The activated or derivatized ion-exchange polymer salts can be combined with thermoplastic or thermoset polymer precursors to generate biologically activated polymer composite mixtures, including moldable, extrudable, layerable and paintable, activated polymer composite mixtures. These mixtures can be hardened or cured to make uniquely surface activated hardened polymer composite materials, coatings and components of textiles, devices, furnishings and apparatus, among other products.

Primary compositions of the invention comprise activated ion-exchange materials typically provided in the form of polymer salts, including activated salts of polymer resins (insoluble cross-linked polymers). Suitable ion-exchange polymers include cation-exchange polymers as well as anion-exchange polymers. The polymer salts incorporate one or more biologically active ionic agents, for example an ionic or ionizable antimicrobial agent (for example an ionic antimicrobial such as an oligodynamic metal, or ionic antibiotic, or an antimicrobial converted to an ionic form by chemical modification.

A wide assemblage of ionic or ionizable antimicrobial agents can be incorporated in activated polymer salts of the invention, including antibacterial drugs, antibiotics, antiviral agents, antifungal agents, organometallic compounds, and oligodynamic metals. Other useful biologically active agents within the methods and compositions of the invention include antiseptics, antimycotics, anti-inflammatory agents, antiproliferative agents, antineoplastic agents, chemotherapeutic agents, antihypertensive agents, anti-arrhythmic agents, anticoagulants, antioxidants, antiparasitic agents, anticonvulsant agents, antimalarial agents, amine-containing pharmaceutical agents, and other therapeutic agents obtainable in ionic form for use within the compositions and methods of the invention.

Biologically active ionic or ionizable agents are captured or bound in an insoluble matrix by ionic association with ion-exchange polymers, often cation or anion-exchange polymer “resins.” Useful ion-exchange polymers are often insoluble in non-ionic aqueous media (e.g., distilled water and alcohols). In some embodiments the ion-exchange polymer may be insoluble or poorly soluble in non-ionic and ionic aqueous media. Associated with aqueous solubility, the subject ion-exchange polymers often possess hydrophobic character, e.g., as is true for polymers crosslinked with bifunctional hydrocarbon monomers (such as divinylbenzene), where both ionic and non-ionic aqueous media will not substantially wet or saturate the ion-exchange polymer (typically wetting or hydration potential will be marked by less than 25% water saturability by weight of the material soaked in aqueous media, often less than 20%, less than 10%, or less than 5% w/w). Hydrophobicity and hydrophilicity are, in part, related to the relative amount of crosslinking of the ion-exchange system and as such can be adjusted for different materials and uses according to the teachings herein (e.g., by altering the ionic component of the system, crosslink density, and/or counter-ion bound to the resin system).

Useful cation-exchange materials for constructing biologically activated polymer salts may include weak or strong cation-exchange materials. Weak cation-exchangers may contain, for example, carboxyl (—CO₂⁻) functionalities (alternatively “moieties,” or terminal or side functional groups). Strong cation-exchangers are exemplified by sulfonates (—SO₃⁻). In general, carboxylates have lower binding constants than do their strong cation-exchanging counterparts, such as sulfonates. Accordingly, carboxylates will give up (exchange, release or allow dissociation of) dications such as copper (II) and monocations such as silver (I) more readily than more electronegative functionalities (e.g., sulfonates).

In illustrative embodiments of the invention, strong cation-exchange materials are constructed comprising polysulfonated salts of polymerized styrene (polystyrene). In other illustrative embodiments, polyphosphorylated materials such as cellulose phosphate or phosphates of synthetic organic structures are constructed. These polymeric ion-exchange materials, such as those based upon polystyrene, may be cross linked with divinylbenzene to form insoluble styrene-divinylbenzene copolymer materials with varying degrees of solubility and hydrophilicity (water loving character) depending upon the amount of cross linking agent included. These materials can be crosslinked to form insoluble ion-exchange materials. Exemplary cross linking agents include, but are not limited to diacrylates to form acrylic-co-diacrylate copolymers or divinyl compounds such as divinylbenzene to form acrylic-co-divinylbenzene copolymers. Weak cation-exchange materials are also provided, exemplified by polycarboxylic acid materials (salts or protonated form) that may be acrylic structures formed by polymerization of acrylate materials. In alternative embodiments, cation-exchange materials can include any of a diverse selection of polymers, including styrene, acrylic, vinyl, sulfonate, carboxylate, and phosphate, among others. A variety of initial counter cations can be associated with the ion-exchange polymer base or scaffold, including for example sodium ions, potassium ions, and hydrogen ions.

Thus in different exemplary embodiments of the invention cation-exchange resins are primarily functionalized as polysulfonated salts, polycarboxylated salts, or polyphosphorylated salts. In some embodiments, the ion-exchange polymer will include two or more different polymer salts. Exemplary ion-exchange polymer mixtures include blends of polysulfonates, polycarboxylates, or polyphosphonates. These can be biologically activated by salt exchange according to the methods herein with any of a diverse selection of cationic biologically active agents, for example oligodynamic metal cations, organic cations, or mixtures of organic cations and metal cations.

Anion-exchange materials can include strongly basic or weakly basic anion-exchange materials. Strongly basic anion-exchange materials generally include poly(quaternary ammonium ion) salts and weakly basic anion-exchange materials generally include polyamines that are generally secondary amine structures but can include tertiary amines as well. These ion-exchange materials can be copolymers of styrene and divinylbenzene, sometimes referred to as styrene-divinylbenzene copolymers. In some embodiments, anion-exchange materials can include polymers such as styrene, vinyl, amine, quaternary ammonium as well as counter anions such as chloride ion and hydroxide ion, for example.

The anion- or cation-exchange materials may be functionalized as described and ionically bound to one or more biologically active ionic agents that possess a distinct biological activity (which may comprise a specific therapeutic efficacy, such as an antimicrobial or anti-inflammatory activity). Useful biologically active ionic agents include any biologically active agent (e.g., an antimicrobial or anti-inflammatory agent) that can be prepared in an ionic form, such as an ionizable salt form. The biologically active agent is loaded onto the ion-exchange polymer typically as a substitute counter-ion by ion-exchange to replace an initial counter-ion (e.g., Na+) and form a new, biologically activated polymer salt. The biologically active replacement counter-ion can include any of a diverse selection of ionic or ionizable agents having a desired biological or therapeutic activity, including for example one or more of a metal cation, quaternary ammonium compound, organic ion, protonated amine, carboxylate, phosphate, cation or anion surfactant, and/or a biguanide. In exemplary embodiments, the counter-ion material can include one or more mono, di, and/or trivalent cation(s). Exemplary metal cations include, but are not limited to, Na⁺, Ag⁺, Au⁺, Cu⁺⁺, Ga+⁺⁺, Zn⁺⁺, and Ce⁺⁺⁺, Fe⁺⁺, and/or combinations thereof. Exemplary quaternary ammoniums include, but are not limited to, benzalkonium chloride, cetrimonium (cetrimide) chloride, and cetylpyridinium chloride. Exemplary protonated amines include, but are not limited to doxycycline hydrochloride, minocycline hydrochloride, Exemplary biguanides include, but are not limited to chlorhexidine diacetate, metformin, proguanil, and chlorproguanil.

Useful biologically active cationic and anionic agents for binding to ion-exchange polymer materials include, but are not limited to, antimicrobial compounds including oligodynamic metal ions, charged pharmaceutical agents including therapeutic agents or drugs effective in the treatment and care of multicellular organisms, and other ionic substances that can be improve the improve a particular clinical or biological environment. Among exemplary antimicrobial agents illustrated here are antibacterial drugs, including antibiotics, antiviral agents, anticoagulants, antifungal agents, organometallic compounds, antiparasitic drugs, as well as oligodynamic metals. Exemplary therapeutic agents include, but are not limited to, anti-inflammatory agents, chemotherapeutic agents, antibiotics, antioxidants, antimalarials, contraceptive agents including spermicidal agents, amine-containing pharmaceutical agents and the like.

Useful ion-exchange polymer materials for association with biologically active ionic agents may be soluble or insoluble. In some embodiments, the ion-exchange polymer material is an insoluble matrix or support polymer, which can take the form of small particles or beads on the order of millimeters in diameter. Exemplary ion-exchange resin materials of this type desirably possess porous particulate structures, with pores on the surfaces and channels and voids communicating with the surfaces of the resin particles. This porous construction enhances ion-exchange functionality of the resin particles (i.e., it increases ability of the particles to communicate with and exchange biologically active ions for original counter-ions associated with the resin material from which the particle is formed).

Exemplary ion-exchange polymers for use within the invention include styrene, acrylic, vinyl, polymethacrylic acid divinyl benzene, and/or polyalkalines, among others. In certain embodiments, the ion-exchange polymer is cross-linked to modify solubility and ion-exchange potential. Exemplary cross-linked polymers include, but are not limited to, polyarylenevinylsulfonate, polystyrene-sulfonate, polyvinylsulfonate, polyalkylenesulfonate polyantholesulfonate, and/or acrylamidomethyl propane sulfonate polymer. In one exemplary embodiment, a polystyrene is employed that is variably or adjustably crosslinked through addition of 0.1-55 mole % of divinylbenzene:styrene during polymer polymerization—to create a range of selectable strength ion-exchange capacity, loading potential (i.e., selectable total load capacity of biologically active counter-ion) and optionally a variable potential for dissociating the biologically active ion for drug delivery purposes (e.g., when in contact with physiological, ionic fluids and tissues).

Ion-exchange polymer materials for use within the invention are generally functionalized to bind or tightly associate ionically with cations or anions. For example, acrylics, styrenes and polyalkylenes may be functionalized by binding to one or more sulfonate, carboxylate and/or phosphorylate ions—to form such exemplary useful polymers as arylenevinyl sulfonate, styrene sulfonate, vinyl sulfonate, or divinyl benzene. These polymers will typically be employed in a first (unactivated) polymer salt form, lacking the “biologically active ionic agent”, and instead having an inactive, “initial counter-ion” present to exchange with the biologically active agent, such as sodium (Na+).

Functionalized ion-exchange materials are often provided in the form of a “first ion-exchange polymer salt”, for example sodium polystyrene sulfonate. Illustrating general “salt exchange” designs contemplated here, the Na+ ionic component of the first polymer salt (sodium polystyrene sulfonate (a polymetallosulfonate)), can be exchanged with any of a variety of biologically active (e.g., antimicrobial or therapeutically effective) metal cations to prepare mono, di, tri, and even tetravalent metal ion, “biologically activated polymer salt” derivatives. Similarly, polymetallosulfonates such as sodium polystyrene sulfonate can be converted to a polyorganosulfonate derivative (e.g., by exchange of sodium for any nitrogen atom containing salt/protonatable nitrogen compound of interest). Exemplary nitrogen atom containing salts/protonatable nitrogen compounds for use in these aspects of the invention include amines, ammonium ions, amidines, amidinium ions, imines (iminium ions), thiazoles, imidazoles, guanidines, guanidinium ions, and/or pyridines, and pyridinium ions. In other illustrative embodiments, ammonium ion-exchange polymer salt derivatives can be produced by exposing amino compounds to acid forms of polymers, for example and acid form of polysulfonate.

To produce primary biomaterials of the invention comprising activated ion-exchange polymer salts, ion-exchange polymers are associated with biologically active counter-ions as shown in Reaction scheme 1. In this scheme an exemplary polysulfonated material is used, where Cat^{m+} is an organic or oligodynamic metal cation. R is a carbon containing group, m=z(q), where z and q are whole numbers greater than 1, n is a number greater than 1, and X is a counter-ion.

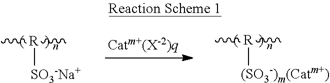

The (R)ₙ is any oligomeric or polymeric backbone. The R group may include monomers such as arylenevinyl sulfonate, styrene sulfonate, divinyl benzene, and/or vinyl sulfonate monomers as well as nonsulfonated monomers. In some embodiments, the oligomer or polymer can include repeating units of the same monomer or a plurality of different monomers. The oligomer may be copolymerized with monomers and/or other oligomers to form a co-polymer. For example, the polymer backbone of polysulfonated cetylpyridinium salt may be polyarylenevinylsulfonate, polystyrene-sulfonate, polyvinylsulfonate, polyantholesulfonate, and/or acrylamidomethyl propane sulfonate polymer. In other embodiments a co-monomer may serve to crosslink the polymer to increase stability and decrease solubility or hydrophilic character.

In exemplary constructions of activated ion-exchange polymer salts, the initial ion-exchange polymer may be selected from a commercially available polymer, for example a commercially supplied polysulfonated resin, such as Amberlite™ IRP69 (Sodium Polystyrene Sulfonate USP, an insoluble, strongly acidic, sodium form cation-exchange resin supplied as a dry fine powder) or Amberlite™ IR88F (Polacrillin Potassium NF, a weakly acidic potassium form cation-exchange resin supplied as a dry fine powder).

Insoluble ion-exchange materials can be created by cross-linking. At lower levels of cross linking (produced with a lower concentration of cross linking agent), the polymer may possess some hydrogel-like character, whereas at higher crosslink densities the absorption of water is minimized and solubility of the resin material is reduced to a point generally recognized in the art as “insoluble”. In exemplary embodiments, insoluble polysulfonated ion-exchange materials are created by addition/copolymerization of a vinyl derivative, such as styrene, with a di- or tri-functional cross linking agent such as divinyl benzene. In this and similar examples, the ion-exchange material, will typically have a crosslinking unit density or concentration in a range of between 0.1 and 20 mole percent, which will generally be correlated with a desired ion-exchange capacity of the resin. Desired ion-exchange capacities found useful for production of activated ion-exchange polymer salts of the invention will typically range between 0.1 and 20.0 mEq/gram.

In certain aspects of the invention, ion-exchange polymer salts are provided in particulate form before activation by salt exchange (i.e., to exchange the initial, inactive counter-ion with a biologically active ionic agent to form the activated ion-exchange polymer salt). Suitable particles sizes of ion-exchange polymers for preparation of activated polymer salts by salt exchange (to form biologically activated ion-exchange polymer salt particles) will often have an average particle size or diameter in a range of a conventional ion-exchange, for example from about 0.05 mm to about 2.5 mm, about 0.05 mm to about 1.5 mm, or about 0.075 mm to about 0.5 mm. In other embodiments, the particle size diameter of the ion-exchange polymer starting material will be from about 300-500 μm, or about 500-700 μm.

Exemplary ion-exchange polymer salts employ a polymer matrix that is effectively water insoluble. Insolubility as used here means that essentially all (at least 95-98%) of the subject polymer material remains insoluble (e.g., precipitated) in deionized water at room temperature. Generally the polymer matrix will remain insoluble even in ionic solutions, such as saline or physiological fluids. In illustrative embodiments, sodium polystyrene sulfonate and poly(vinyl carboxylic acid), otherwise known as polyacrylic acid, sodium salts are water soluble materials. These and like materials can be rendered more or less insoluble for use within different aspects of the invention by variable cross-linking, as described. One exemplary useful commercial product Amberlite IRP69, (Rohm and Haas Company, a subsidiary of Dow Chemical Company, Philadelphia, Pa. 19106-2399), is a sulfonated styrene co-divinyl benzene (crosslinked) ion-exchange resin. Another exemplary commercial product for use within the invention is Amberlite IRP64, (Rohm and Haas Co.) a polymethacrylic acid, co-divinylbenzene (crosslinked) ion-exchange material. Both of these materials are essentially insoluble in water (by virtue of the divinylbenzene crosslinking of the polymers). Generally, the percentage of crosslinking agent is represented as mol %, however it may also be presented as wt % and by potential for swelling by water absorption. Ion-exchange capacity, hydrophobicity and insolubility are all generally directly proportional to amount or percentage of cross linking. By using greater or lesser percentages of cross linking, ion-exchange capacity of polymers within the invention can be varied, as can potential for water absorption and for “reversible association” of loaded biologically active counter-ions (i.e., the potential to release the counter-ion from the activated (loaded) polymer salt into an aqueous medium or ionic fluid or tissue compartment by ionic dissociation). Cross-linked ion-exchange polymer salts are thermally stable, allowing for drying under vacuum at elevated temperatures (e.g., up to 150° C.).

Activating soluble and insoluble ion-exchange materials with biologically active ionic agents (for illustration here, a cation (Cat)^{m+}) results in materials with two different types of solubility behavior. For example, an IRP69 modified product can release (Cat)^{m+}(X⁺)ₘ in the presence of salt solutions such as NaCl (Na⁺X⁻) such as saline or physiologic fluids. The exchange reaction between sodium polystyrene sulfonate and an organic cation salt or a multivalent metal cation may produce an insoluble salt such as with doxycycline:HCl or gallium nitrate when prepared in deionized water. However upon exposure to saline solution the complex salt can dissolve thus liberating sodium polystyrene sulfonate and doxycycline:HCl and in the case of gallium polystyrene sulfonate, gallium chloride (GaCl₃) and sodium polystyrene sulfonate.

Activating an initial ion-exchange polymer salt by salt exchange to substitute biologically active ionic agents (including metallic and organic ionic or ionized compounds) may be aided by addition of heat or pressure, by the use of columnar flow reactors, and use of various solvents, as elsewhere described.

As shown in illustrative Reaction Scheme 1, the number of positive charges in the depicted ion-exchange polymer material is equivalent to the number of sulfonate groups present in the exemplary polymer. Accordingly, if the cation is a di-cation for example, it can be associated with more than a single sulfonate, carboxylate or phosphorylate group. In one exemplary embodiment, sodium polystyrene sulfonate is associated with an oligodynamic metal as shown in reaction scheme 2.

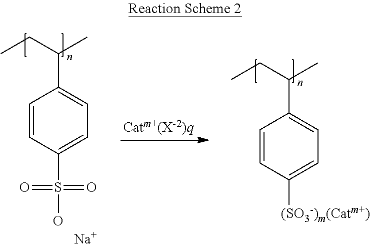

In another exemplary embodiment, polystyrene sulfonic acid-co-divinylbenzene is combined with the acetate salt form of an organic or metal cation (e.g. silver acetate or copper (II) acetate) in deionized water. The byproduct odor of acetic acid is evidence that the reaction has proceeded to yield the metal or organic sulfonate. With completion of this reaction the product can be titrated to determine residual sulfonic acid content which is an indicator of the degree of substitution on the resin backbone and an indicator of yield. Mass balance can provide corroborating yield data. In the event that residual acid is present in the final product the resin can be detrimental to any matrix it may be incorporated into as a result of acid-mediated hydrolysis or degradation of the matrix. Residual acid in the activated polymer salt resin was determined to cause degradation of the resin during attempts to dry the product after synthesis. Furthermore, residual sulfonic acid was observed to catalyze the formation of ethers when combined with hydroxyl compounds such as isopropanol or ethanol. To avert this problem it is often useful to back-titrate mixed metal/organic sulfonic acid resins and other resins modified to incorporate ionic active agents, for example using sodium acetate or another appropriate acetate salt to prepare acid-free mixed ion resins that did not react with hydroxyl compounds or cause degradation of the polymer matrices they were formulated into.

These studies reveal unexpected results useful for synthesizing novel resin biocides in high yield, and in stable, adjustable and/or titratable forms, for safe incorporation into a variety of activated polymer composites. It is important to note that all sulfonic acid residues must be completely converted to salt form in order to utilize them for incorporation in a polymer matrix. Optimally the subject manufacturing stages for the construction of activated polymer salts are conducted using wet or at least non-dried materials, as drying results in degradation as described (e.g., as observed by mass loss, darkening of the resin, odor and presence of extractable acid). Further, the exchange capacity is optimally characterized in detail, for example by calculating exchange capacity of wet sulfonic acid resin using titration, as described. This ensures proper stochiometry, e.g., as with respect to incorporation of exemplary acetate salt. In another exemplary embodiment, the acid form of polymethacrylic acid-co-divinylbenzene can be reacted with an acetate salt of a metal or organic cation to yield byproduct acetic acid and the metal or organic salt of the methacrylic acid copolymer. The biologically active, exchanged counter-ions can be variably loaded, e.g., for titrated activation and/or selected or metered release kinetics, onto the polymer backbone to finely adjust surface activity properties of the materials and products herein, for example by associating a selectable load concentration or density of from 1% to 100%, often 5-80%, 10-50%, in some embodiments from 20-30% (of available exchange sites loaded with active agent), to calibrate loading and ultimate surface activity and release kinetics (of the biologically active, substituting counter-ion with functionalizing ion, e.g., sulfonate) on the polymer backbone. Typically “non-loaded” functionalizing ions remaining associated with the original counter-ion (e.g., Na+). In other aspects of the invention, a polymer backbone for constructing activated polymer salts may be modified to include more than one active ion, for example cetylpyridinium(+) and silver(+), or any other combination of multiple active ions identified herein.

At least a portion of the biologically activated ion-exchange polymer salt particles produced here will typically retain at least some “non-loaded” functionalizing ions. In other words, the biologically active exchange counter-ion will not be associated with the polymer at all available ion-exchangeable sites. Even while the loading of these sites with biologically active counter-ion is “variable” or “selectable” using ion-exchange chemistry methods described here, the maximum loading of fully exchange biologically activated polymer salts will typically be less than or about 90% absolute ion-exchange saturation capacity of the ion-exchange polymer (e.g., 90% replacement of total available initial counter-ion, such as Na+, substituted by the biologically active counter-ion, e.g., Ag+). Expressed alternatively, the maximal loading of biologically active counter-ion onto selected ion-exchange materials will often range no higher than 90% of a theoretical maximum ion-exchange capacity of the subject ion-exchange material. Within this range the materials and products of the invention can be finely tuned for selected levels of biological activity (depending mostly on the agent and specific biological activity being employed) by metered loading of the ionic active agent onto the polymer backbone. For different activities where a lower “dosage” or loading is desired (e.g., when the biologically active ionic agent is particularly potent, or perhaps toxic, or when materials may be used in contact with sensitive tissues), the loading may be reduced to a minimum level (for example where only 1-5% or 1-10% of the projected ion-exchange potential of a polymer is occupied, or where only 1-5% or 1-10% of initial counter-ions (e.g., Na+) available in the ion-exchange polymer are replaced by the biologically active substitute counter-ion (e.g., Ag+). For intermediate potency biologically active ionic agents, intermediate ion-exchange polymer loading levels may be selected of between 10-25%, 25-45%, 45-65% or higher. For low potency biologically active ionic agents, or more resistant targets, higher titer loading is employed, for example in ranges of 50-70% (e.g., % of maximum ion-exchange potential, or % of initial counter-ions actually exchanged by biologically active substitute counter-ion), 70-85%, 85-90% or even higher (up to practical saturation).

Variable loading of ion-exchange polymers with one or more biologically active counter-ions to make biologically activated polymer salts often involves use of ionic salt solutions having selectable concentrations (higher for higher targeted loading), and use of other variable conditions (e.g., varying temperature and/or pH, use of other solvents in addition to water, addition of other salts, etc.) conventionally used for ion-exchange. Also considered in this context are differences in ion-exchange capacity, for example cation-exchange capacity (CEC), of a selected polymer. CEC represents the maximum quantity of total cations of any class that a polymer is capable of holding at a given pH value. CEC can be expressed as milliequivalent (mEq) of cation per gram or per 100 grams (mEq/g or mEq/100 g) of ion-exchange material. One insoluble ion-exchange material employed here for illustrative purposes, Amberlite IRP69, has an estimated CEC on the order of 4.6 mEq/g. In one exemplary ion-exchanged IRP69 resin, where sodium counter cation is replaced with silver, a final “activated polymer salt” composition incorporates approximately 37% Ag by weight. Increasing the silver titer or load of this exemplary construct to 100% of the theoretical maximum exchange capacity of silver ion for sodium, would yield a maximally activated polymer salt comprising roughly 53% silver by weight. Employing the variable loading methods and materials described here, the invention provides for variable loading of ion-exchange polymer salts with different oligodynamic metals to yield variably loaded ion-exchange polymer salts comprising 1-10%, 10-20%, 20-30%, 40-50%, or greater, up to practical saturation, of the oligodynamic metal ion by weight.

In some embodiments, the functionalizing ion in the ion-exchange material may be carboxylate. As shown in Reaction Scheme 3

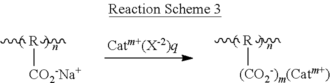

In some embodiments, the polycarboxylated salt may possess an exchange capacity of between 1.0 and 15.0 mEq/gram. The polycarboxylated salt may comprise one or more monomers of acrylic acid, methacrylic acid, vinylbicenzoic acid, arylenevinyl carboxylate, or divinyl benzene. In exemplary embodiments, the polycarboxylated ion-exchange resin may be a commercially available product such as Ambcrlite™ IRP64 (Polacrilix resin) or Dow Chemical's MAC-3 resin systems (polyacrylic). Amberlite IRP64 has a reported exchange capacity of 10.0 mEq/g. Biologically active counter-ions can be associated with one or more of the carboxylate groups in the ion-exchange polymer. In some embodiments, the composition may include a blend of at least two different salts of a polycarboxylated compound, whereby the cation occupies from 1% to 100% of available carboxylates. In more detailed embodiments, the biologically active cation or anion occupies from 1-10%, 10-20%, 20-40%, 40-60%, 60-80% or 80-95%, up to between 90% and complete practical saturation, of available functional groups/exchange sites as active counter-ion within the activated ion-exchange polymer salt.

In some embodiments, the functionalizing ion in the ion-exchange material may be phosphate. An exemplary cation-exchange polymer of this type is cellulose phosphate. This material can be activated by antimicrobial cations such as copper (II), for example. Cellulose phosphate is a strong cation-exchange material of variable ion-exchange capacity (generally around 7 mEq/gram).

Biologically active counter-ions for activating ion-exchange polymer materials can include any number of inorganic or organic cations or anions. Counter-ions that can be readily associated (without chemical conversion to an ionic or salt form) with useful ion-exchange polymers can include one or more metal cations, organic cations, quaternary ammonium compounds, protonated amines, carboxylates, phosphates, amine containing therapeutic agents, ammonium containing antibiotics and antimicrobial agents, nitrogen containing antibiotics and/or biguanides.

In some embodiments, the cationic or anionic biologically active agent is a mono, di, or trivalent metal including, but not limited to, an oligodynamic metal cation such as silver(I)/Ag(II), copper(II)/Cu(II), zinc(II)/Zn(II), iron(II)/Fe(III), gallium/Ga or bismuth(II)/Bi(II), amenable to ionic association with a sulfonate, carboxylate, or phosphate anion, for example. In other examples, the metal cation may be one or more of a monocationic species Na⁺, Ag⁺, K⁺, Li⁺, Au⁺, a dicationic species Ba++, Ca⁺⁺, Cu⁺⁺, Zn⁺⁺, Mn++, Mg⁺⁺, Fe⁺⁺, or a trication species such as Bi⁺⁺⁺, Ga⁺⁺⁺, and/or Ce⁺⁺⁺ or combinations thereof. In illustrative embodiments, useful materials for association by counter-ion-exchange with an ion-exchange polymer salt (e.g., a polysulfonated resin salt), include, for example, a silver salt, copper (II) salt, cerium (III) salt, Gallium (III) salt, cetylpyridinium salt, benzalkonium salt, chlorhexidine salt, centrimonium (centrimide) salt, octenidine salt, zinc (II) salt, iron (II) salt, or minocycline salt, or combinations thereof, as shown in the exemplary structural diagrams below.

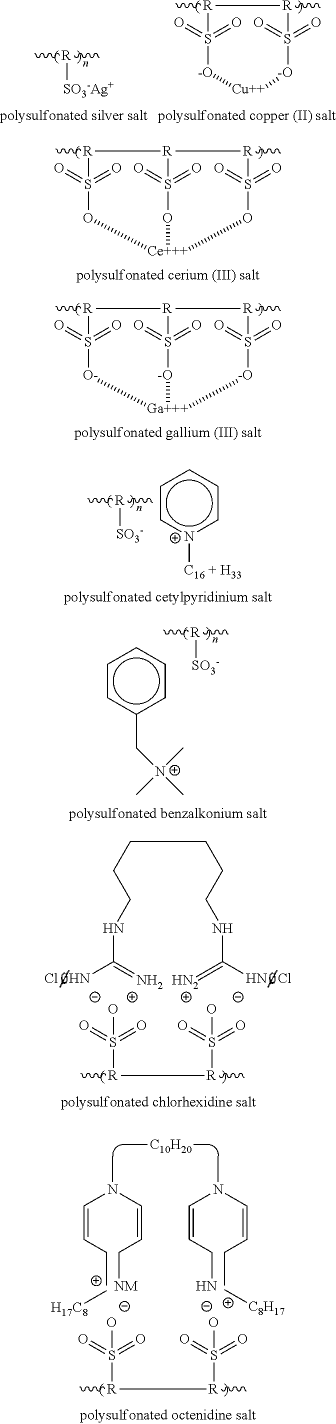

The sulfonate may also be associated with NH₄⁺, RNH₃⁺, R R″NH₂⁺, RR′R″NH⁻, or⁺, RR′R″R′″N⁺, for example where R represents an aryl, alkyl or mixed aryl alkyl groups or the sulfonate can be associated with a pyridinium cation. According to another example, the sulfonate group can be associated with one or more of organic species including nitrogen containing organic species such as an amino acid, a tetracycline, doxycycline, arginine, gentamycin, ammonium chloride, cetyltrimethylammonium bromide, lysine, glutathione, lidocaine, albuterol, and/or alkyl/benzylammonium, pyridinium such as cetyl pyridinium, a guanidinium ion such as with chlorhexidine or polyhexanide, amino or oxazole, triazole, or thiazole containing compounds such as antifungal agents to include ketoconazole, or clotrimazole, and (dihydropyridinyl) species such as octenidine for example.

In some embodiments, the copolymeric ion-exchange material may be ionically bound to a plurality of therapeutically useful counter-ions. For example, an oligodynamic metal ion and a quaternary ammonium ion may both be bound to the same copolymeric ion-exchange material. In other embodiments, more than one therapeutically useful counter-ion from the same class may be bound to the same copolymeric ion-exchange material, for example a plurality of two oligodynamic metal ions may be bound to the same copolymeric ion-exchange materials as shown with copper and zinc in Table 1.

TABLE 1
ICP/MS Results from IRP69-Cu/Zn (binary on same resin)

| | Concentration | Concentration |
|---|---|---|
| | Cu | Zn |
| True Conc Zn + Cu in sample | 11494812 | 9459786 |
| (ppb) | | |
| Conc in ppm | 11494.81 | 9459.786 |
| Mass (g) | 0.149433 | 0.122977 |
| Mols | 0.002372 | 0.001892 |

Useful antimicrobial counter-ion materials in the self-disinfecting compositions described herein include, but are not limited to, antibacterial drugs, including antibiotics, antiviral agents, antifungal agents, organometallic compounds, antiparasitic drugs, as well as oligodynamic metals.

Useful antibiotics include, but are not limited to, natively cationic antibiotic and antibiotics that are readily protonated to cationic form (each of which can be readily associated with either polycarboxylated, polyphosphorylated, and/or polysulfonated ion-exchange polymers). Exemplary antibiotics include semisynthetic penicillin such as ampicillin and amoxicillin; monobactams such as aztreonam; carboxypenems such as imipenem; aminoglycosides including streptomycin; gentamicin; glycopeptides such as vancomycin: lincomycins including clindamycin; macrolides such as erythromycin; polypeptides such as polymyxin; bacitracin; polyenes such as amphotericin; nystatin; rifamycins such as rifampicin; tetracyclines; and doxycycline, among others.

Exemplary antiviral agents include, but are not limited to, acyclovir, idoxuridine, etravirine, and tromantadine.

Exemplary antifungal agents include, but are not limited to, miconazole, ketoconazole, fluconazole, itaconazole, econazole, terconazole, oxyconazole, grisefulvin, clotrimezole, naftifine, and polyenes such as amphotericin B or nystatin/mycostatin.

Exemplary anti-amoebics include, but are not limited to, metronidazole and tinidazole.

Exemplary antihistamines include, but are not limited to, diphenylhydramine, chlorpromazine, pyrilamine and phenyltoloxamine.

Useful antioxidant counter-ion materials include, but are not limited to, glutathione and carnosine.

Other therapeutically useful counter-ions may comprise ionic or ionizable chemotherapeutic and anticancer agents, such anthracycline antibiotics (e.g., doxorubicin) which can be readily associated with ion-exchange resins of the invention and incorporated in useful composites effective to treat cancer (e.g., by implantation or other delivery of the composite to a site of a tumor). Other useful biologically active agents for use as active ionic (or ionized) agents within the polymer salts and polymer composites of the invention chemotherapeutics include, for example, alkaloids such as morphine, ephedrine, berberine (antibacterial), and caffeine; antihypertensive agents such as verapamil and nifedipine; anxiolytics; sedatives and hypnotics (such as benzodiazepines, diazepam, nitrazepan, flurazepam, estazolam, flunitrazepam, triazolam, alprazolam, midazolam, temazepam, lormetazepam, brotizolam, clobazam, clonazepam, lorazepam and oxazepam); anti-migraine agents such as sumatriptan; anti-motion sickness agents (such as cinnarizine); anti-emetics (such as ondansetron, tropisetron and granisetrone); adrenergics such as amphetamine: antispasmodics (such as aminopentamide, metixene, papaverine, ethaverine and dicyclomine); ataractics such as benactyzine; antihypertensives such as hexamethonium and pentamethonium; analgesics such as 2,6-diamino-3-phenyl-azopyridine and morphine; antitussives (such as dihydrocodeine, phenylpropenolamine, guaiacol, cloperastine and dextromorphen); bronchodilators such as dimethylephedrine; antipsychotics such as imipramine; coronary dilators such as etafenone; antiarrhythmics such as procainamide; hypotensives such as hydralazine and clonidine; and peripheral vasoconstrictors such as tolazoline, among others.

Further examples of amine-containing drug compounds useful within the activated polymer salts and polymer composites of the invention include, for example, acetophenazine, amitriptyline, bromopheniramine, carbinoxamine, chlorcyclizine, cyclizine, desipramine, dexbrompheniramine, dexchlorpheniramine, ergotamine, nortriptyline, quinidine, benztropine, flunarizine, fluphenazine, hydroxychloroquine, hydroxyzine, meclizine, mesoridine, methdilazine, methysergide, pheniramine, pyrilamine, tripelennamine, triprolidine, promazine and quinidine as well as compounds containing functional groups such as a pyrolidine, atropine, pyrrolizidine, quinolizidine, indolizidine, pyridine, isoquinoline, oxazole, thiazole, quinazoline, acridine, quinolone, indole, imidazole, purine, phenethylamine, muscarine, benzylamine, spermine, or spermidine, antihypertensive agents such as verapamil and nifedipine.

Other exemplary compounds with ionizable nitrogen atoms within the structure include: methotrexate; adriamycin; cytosine arabinoside; arabinosyl adenine; PAM: I-PAM; phenylalanine mustard); procarbazine dactinomycin (actinomycin d); mitomycin; aminoglutethimide; estramustine; leuprolide; tamoxifen; amsacrine (m-AMSA); adriamycin; arabinosyl; procarbazine; and dacarbazine; nitrogen mustards: chlorambucil; cisplatin; oxaliplatin; BBR3464: dacarbazine; mechlorethamine; procarbazine; temozolomide; uramustine; methotrexate; pemetrexed; raltitrexed; cladribine; clofarabine; fludarabine; mercaptopurine; thioguanine; capecitabine; cytarabine; gemcitabine; vinblastine; vincristine; vindesine; vinorelbine; daunorubicin; doxorubicin; epirubicin; idarubicin; mitoxantrone; bleomycin; mitomycin; topotecan; irinotecan; aminolevulinic acid; methyl aminolevulinate; porfimer sodium; verteporfin; dasatinib; erlotinib; gefitinib; imatinib; lapatinib; nilotinib; sorafenib; sunitinib; vandctanib (ZD6474); altretamine, anagrelide, bortezomib, estramustine, pentostatin, alagebrium (3-phenacyl-4,5-dimethylthiazolium, anti-helminthics; antitoxins; antivenins; theophylline; aminophylline; hemin; muramyldipeptide; muramyltripeptide; N-acetyl-muramyl-L-alanyl-D-isoglutamine; ketoconazole; nystatin; flucytosine (5-fc); miconazole; amphotericin B; sulfazecin; cyanocobalamin; amelexanox; glutathione; carnosine; p-aminosalicylic acid; isoniazid; capreomycin; cycloserine; ethambutol; ethionamide; pyrazinamide; rifampin; and streptomycin; acyclovir; amantadine; ribavirin and vidarabine; diltiazem; nifedipine; verapamil; dapsone; chloramphenicol; neomycin; cefaclor; cefadroxil; cephalexin; erythromycin; clindamycin; lincomycin; bacampicillin; carbenicillin; dicloxacillin; cyclacillin; picloxacillin; hetacillin; methicillin; nafcillin; oxacillin; penicillins (G&V); ticarcillin; rifampin; doxycycline; mefenamic acid; oxyphenbutazone; phenylbutazone; piroxicam; sulindac; tolmetin; chloroquine; hydroxychloroquine; metronidazole; quinine; quinidine; meglumine; penicillamine; paregoric; codeine; heroin; methadone; morphine; opium; and papaverine; noscapine; atracurium; gallamine; metocurine; pancuronium; succinylcholine (suxamethonium); tubocurarine; vecuronium; flurazepam; methotrimeprazine; midazolam; temazepam; triazolam; bupivacaine; chloroprocaine; etidocaine; lidocaine; mepivacaine; procaine; marcaine; tetracaine; droperidol; etomidate; fentanyl; ketamine; benzyl trimethyl ammonium, chlorhexidine; amino acids (natural & synthetic); nicotinic acid; nicotinamide, pyridoxine; nuclcosides (purines); thiamine; coenzyme A; pentoxifylline; 3-amino-4-hydroxybutyric acid; 6-diazo-5-oxo-L-norleucine; aceclofenac; acediasulfone; alminoprofen; amfenac; amoxicillin; ampicillin; apalcillin; apicycline; aspoxicillin; azaserine; aztreonam; biapenem; bromfenac; bucillamine; bumadizon; candicidin(s); carbcnicillin; carprofen; carumonam; cefamandole; cefatrizine; cefbuperazone; cefclidin; cefdinir; cefditoren; cefepime; cefetamet; cefixime; cefmenoxime; cefminox; cefodizime; cefonicid; cefoperazone; ceforanide; cefotaxime; cefotetan; cefotiam; cefozopran; cefpimizole; cefpiramide; cefpirome; cefprozil; cefroxadine; ceftazidime; ceftcram; ceftibuten; ceftriaxone; cefuzonam; cephaloglycin; cephalosporin C; cephradine; ciprofloxacin; clinafloxacin; cyclacillin; denopterin; diclofenac; edatrexatc; enfenamic acid; enoxacin; epicillin; etodolac; flomoxef; flufenamic acid; grepafloxacin; hetacillin; imipenem; lomefloxacin; lymecycline; meclofenamic acid; melphalan; mcropenem; moxalactam; mupirocin; mycophenolic acid; nadifloxacin; niflumic acid; norfloxacin; oxaceprol; panipenem; pazufloxacin; penicillin N; pipemidic acid; podophyllinic acid 2-ethylhydrazide; procodazole; pseudocphedrine; ptcropterin; quinacillin; ritipenem; romurtide; S-adenosylmethionine; salazosulfadimidine; sparfloxacin; streptonigrin; succisulfone; sulfachrysoidinc; sulfaloxic acid; teicoplanin; tematloxacin; temocillin; tetracycline; tolfenamic acid; (N-((5-(((1;4-Dihydro-2-methyl-4-oxo-6-quinazolinyl)methyl)methylamino)-2-thienyl)carbonyl)-L-glutamic acid); tosufloxacin; trovafloxacin; doxycycline; mafenide; minocycline; tigemonam; or vancomycin; lucensomycin; natamycin or; 6-diazo-5-oxo-L-norleucine; denopterin; edatrexate; eflomithine; and (N-((5-(((1;4-Dihydro-2-methyl-4-oxo-6-quinazolinyl) methyl) methylamino)-2-thienyl)carbonyl)-L-glutamic acid)-ubenimex.

Exemplary contraceptives include spermicidal agents, anti-motility agents effective to disable spermatozoa flagellar function, anti-ovulation agents, and anti-conception agents, among others. An exemplary spermicidal agent that can be associated with activated polymer salts and thereby integrated in biologically activated polymer composites of the invention include, for example, 3α,7α,12α-Trihydroxy-5β-cholan-24-oic acid sodium salt, Cholalic acid, (common name=cholate), presently used effectively in spermicidal vaginal sponges and other anti-conceptive materials and formulations (optionally in conjunction with benzalkonium chloride).

In certain embodiments, spermicides and/or other contraceptive agents are incorporated in the biologically activated polymer composites of the invention, which are fabricated to provide functionally and/or anatomically formed contraceptive devices. In one exemplary embodiment, intrauterine contraceptive devices (IUiDs) are provided combining an activated polymer composite incorporating a copper derivative associated with an activated polymer salt particulate embedded in the composite (e.g., in a form SO3-, CO2-, OPO3-). Comparable active agents and polymer salts can be readily incorporated in vaginal sponge and cervical diaphragm devices, for example, formed partly or entirely from activated polymer composites of the invention. These novel devices are constructed to deliver spermicidal and/or anti-conceptive ionic copper at a surface of the IUD device, sponge, condom or diaphragm, or in other embodiments to deliver a metered or titered dose (i.e., a spermicidal and/or anti-conceptive effective amount) of solubilized ionic copper to a target site, such as a vaginal, cervical or uterine compartment, to mediate effective contraception (often by activatable dissociation/solubilization of the active ionic agent triggered by contact of the activated composite surface with an ionic, e.g., physiological, fluid). In other embodiments employing fundamental compositions and methods of the invention, vaginal sponges, condoms, cervical diaphragms and IUDs are provided incorporating an anti-conceptive effective amount of benzalkonium and cholalic acid (cholate) in an activated polymer composite. Yet additional vaginal sponges, condoms, cervical diaphragms and IUDs will incorporate an anti-conceptive effective composition comprising an acid form of sulfonated polystyrene divinylbenzene, fabricated in a high surface area activated polymer composite construct (e.g., a sponge, or a lattice-like, blown or open cellular fabricated and/or molded silicone composite) to generate anti-conceptive effective amounts of hydronium at the surface of the device or effectively solubilized to mediate activity away from the composite surface. Notably, benzene sulfonic acid has a dissociation constant of 10³ and thus can effect local pH reduction at targeted compartments, for example a cervical orifice. Because sperm require high pH for conceptive function, these activated composite compositions and constructs will provide highly effective contraceptive materials and devices.

Within other novel embodiments of the invention, biologically activated polymer composites will incorporate ionic or ionizable forms of anticoagulants and other hematologically active compounds useful to prevent blood clotting, inflammation, atherosclerosis, restenosis, stroke and other adverse sequelae associated with vasculary and coronary pathologies, and/or with conventional use of vascular stents, shunts, grafts (artificial and autologous), prostheses or implants (e.g., coronary valves, pacemakers and electrodes). In exemplary embodiments, a low molecular weight heparin such as Dalteparin can be effectively employed within composites of the invention incorporated within these biomaterials and devices as described, to prevent clotting and/or restenosis after vascular or coronary surgery. Additional embodiments will employ Cloricromen, a platelet aggregation inhibitor. Other embodiments will employ Benzamidine-based thrombin inhibitors such as α-NAPAP (N-alpha-(2-naphthylsulfonylglycyl)-4-amidinophenylalanine piperidide). Direct Thrombin Inhibitors such as Dabigatran (Ethyl 3-{[(2-{[(4-{N′-hexyloxycarbonylcarbamimidoyl}phenyl)amino]methyl}-1-methyl-1H-benzimidazol-5-yl)carbonyl](pyridin-2-yl-amino) propanoate) are similarly useful within anti-clotting, anti-sclerotic, anti-thrombotic, anti-restenotic, anti-stroke, anti-arrhythmic, and anti-coronary arrest biomaterials, devices and methods, among other related compositions, implants, apparatus and methods.

Additional operative embodiments of the invention employ peptide-based therapies, with biologically activated composites incorporating ionic or ionizable peptides, peptide fragments, peptide conjugates and other useful peptide drugs and compositions. Peptide drugs can be challenging to deliver given their susceptibility to the gut and to proteases that can degrade activity. Small peptides can be associated with biologically activated ion-exchange polymer salts according to the teachings herein, and these can be formulated within polymer composites in a wide array of useful biomaterials and devices. In one exemplary embodiment, a peptide active agent is incorporated in an activated polymer composite of the invention as a vaginal, colonic or oral sponge, capsule, implant or particulate suspension for delivery of the active peptide to a highly vascularized mucosal tissue of the vagina, uterus, lower colon or rectum, or oral mucosa. Other mucosal peptide delivery forms include nasal delivery composites. In certain embodiments, fine particulate polymer salts alone will be delivered as an active particulate aerosol to carry aerosolized particulates carrying ionic active agents to an intranasal or intrapulmonary target tissue, where the ionic agents may be released (dissociated and solubilized from the polymer salt carrier following contact with physiological ionic fluid) or mediate surface active drug, antimicrobial or therapeutic activity.

In additional aspects of the invention, a wide range of orthopedic biomaterials and devices will beneficially incorporate activated polymer salts and polymer composites of the invention. Among many orthopedic uses contemplated, posts of implants are known to be high-risk conduits for entry of microbial infectious agents into hip implant patients. The invention provides a variety of useful composites to prevent this contamination/infection risk, including epoxy, silicone, and acrylic plugs compounded with sulfonated polystyrene-divinylbenzene-tobramycin or gentamicin salt (or polymethacrylic acid-divinylbenzene-tobramycin or gentamicin salt) for placement at a site of a hip implantation post. These composites and devices provide effective slow release of ionically associated drug over time. In more detailed embodiments, these composites (generally useful for adjunctive use with a diverse array of prosthetic implants, including dental and surgical posts, pins, anchors, sutures, stents, etc.) are often formed as a porous solid composite (e.g., spongiform, lattice form, open cellular, blown or extruded composite), which can be facilitated by addition of any of a variety of known useful polymer foaming agents—to increase surface area for enhanced drug delivery (i.e., with higher kinetics or doses of drug delivered, and more effective sustained delivery—e.g., with effective delivery amounts maintained for 1-3 days or weeks, 1-3 months or longer). Polyurethane-based polymer composites described herein are particularly amenable to fabrication involving foaming, due to the ease forming CO₂ during cure exhibited by these polymers.

Many useful drugs and other therapeutic agents that do not natively exist in an ionic form, or which are not available in a useful salt form to provide for preparation of ion-exchange polymer salts, can be rendered into such useful forms by a variety of chemical processing and/or chemical modification methods. Methods for generating drug forms amenable to binding to anion-exchange polymer materials, for example, include formation of carboxylate (CO₂—) by hydrolysis of esters (CO₂R), where R is generally an alkyl group. In comparably useful processing methods, sulfonates can be generated in the same fashion (although sulfonic acid esters can be alkylating agents and therefore mutagens or carcinogens when encapsulated into a polymer matrix, particularly hydrophilic matrices wherein the sulfonic acid esters can hydrolyze to release a hydroxyl-terminal component of the ester). One method for the delivery of hydroxyl-terminated drugs therapeutic agents) involves the formation of the sulfonic acid ester of a strong cation-exchange resin such as IRP69 (IRP69-SO₂—OR) where OR represents the hydroxyl-terminated active agent. In the presence of water, within a polymer matrix, such as a matrix designed to absorb water (a hydrogel for example), the sulfonic acid ester is hydrolyzed to yield the hydroxyl-terminated active agent (HOR) plus the sulfonic acid of the ion-exchange resin (IRP69-SO₃H). One such active agent for functionalizing the resin is dexamethasone by reaction with the sulfonic acid chloride. Similar chemistry can be applied to phosphate esters as well, as these compounds can be hydrolyzed in similar fashion.

For conversion of active drugs and therapeutics for binding to cation-exchange polymer materials, protonation is a readily practiced modification method (here, quaternization is generally referred to alternatively, as alkylation at nitrogen of principally tertiary amines). Protonation of amino functionalities (primary, secondary, and tertiary amines) by acid forms of ion-exchange polymers (e.g., carboxylic, sulfonic, and phosphoric functionalized ion-exchange polymers) provides a ready and efficient tool for converting target active drugs and therapeutics to render them suitable as polymer salt exchange counter-ions in this fashion. One method that can be applied is the hydrolysis of ester functionalities in order to yield carboxylates so that they may be bound to an anion-exchange resin for example. In other embodiments, hydrolysis catalyzed by an acid functionalized form of ion-exchange polymers provides another broadly applicable tool to convert non-ionic target drugs and therapeutics to useful salt-exchange counter-ions to form activated polymer salts and related composites.

In certain embodiments of the invention, activated ion-exchange polymer salt particles are further processed to achieve size reduction from an original ion-exchange particulate size. Typically this size reduction processing involves fracturing of the original ion-exchange particle, but this can be achieved also by mechanical cutting, shearing, grinding or erosive techniques. Particle fracturing can be achieved using a variety of particle size reduction/milling methods.

Briefly, the starting ion-exchange material (before activation) is generally provided in the form of particles ranging from about 100 μm to about 2,500 μm in average diameter, often in the range of 500 μm to 1,500 μm. In various embodiments, it is desired to achieve substantial size reduction of these particles by milling to generate a fine particulate, activated ion-exchange polymer salt or resin material. Desired size ranges for these materials range from about 10 nm to about 100 μm in average diameter. In certain embodiments the average particle diameter of the fine particulate, activated ion-exchange polymer salt or resin material will be from about 100 nm to about ten μm. In other detailed embodiments the fine particle diameter will range from about 100 to about 700 nm, from about 200 to about 600 nm, and in certain exemplary embodiments about 300 nm, 400 nm, or 500 nm. Desirably, the fine particulate milled, activated ion-exchange polymer salt material will demonstrate a desired uniformity of particle size variation, in some embodiments a Gaussian distribution of particle size variability (e.g., as determined by laser particle analysis).

While different methods, apparatus and compositions for milling may be used for different embodiments and aspects of the invention, one exemplary mode of milling of the activated ion-exchange polymer salt particles employs high energy milling, for example using centrifugal/planetary ball milling methods, compositions and devices. Within more detailed embodiments, high-energy milling is combined with a porous construction design of the ion-exchange polymer salt particles prior to milling. In exemplary embodiments, ion-exchange polymer salt particles may be provided with a microporous construction, wherein individual particles define small channels, voids and pore spaces within the body of the resin particle (the pore spaces and channels being surrounded by walls or partitions of the polymer salt material). After the porous polymer salt particles have been biologically activated by salt exchange with the biologically active ionic agent in aqueous media, the particles are dried to remove some or all of the water present in advance of milling. Subsequently the biologically activated porous ion-exchange polymer salt particles are milled by a high energy milling process to render the fine particulate biologically activated ion-exchange polymer salt particles as described.

In exemplary embodiments, high energy milling of activated, porous ion-exchange polymer salt particles is conducted in the presence of a non-solvent liquid. The non-solvent liquid is added to occupy channel, void and pore spaces within the polymer salt particles. It has been discovered here that using these novel high energy milling materials and methods, the non-solvent liquid mediates size reduction of the polymer salt particles in an unexpectedly efficient and uniform manner. This is effected by the non-solvent liquid providing compression resistance against interior surfaces of the particle walls and partitions, which opposes pressure and mechanical forces exerted on the opposite surface of a wall or partition (e.g., an “external” surface of a wall defining a void space, or an opposite surface of a wall flooring a pore, or dividing two void spaces or channels filled with the non-solvent liquid (in contact with the internal or “facing” surface of the wall or partition). This compression resistance enhances efficiency and uniformity of particle size reduction during milling by facilitating structural failure or fracture of the walls and partitions throughout the porous polymer salt particle. This failure or fracture mediated by shear, pressure and other mechanical forces imparted by the milling has been determined to be facilitated by the presence of the non-solvent medium, and without the medium fracture/failure would be less efficient and uniform due to elasticity and compressibility of the porous particle walls and partitions. The resulting product of this and equivalent high energy milling processes and formulae provided by the invention, is a novel, fine particulate biologically activated ion-exchange polymer salt material, having an average milled particle diameter between about 10 nm to 100 μm, often as small and uniform as from 100 nm to 10 μm, and in certain embodiments ranging between about 400 nm to 600 nm (for example having an average fine particle diameter of 500 nm).

In one illustrative milling protocol provided here, larger porous activated ion-exchange polymer salt particles are placed into a stainless steel container lined with a hard ceramic, such as zirconium oxide. A non-reactive organic liquid (for example heptane or octane) and suitable milling media (for example barrel- or ball-shaped, ceramic milling media, such as zirconium oxide bearings) are added to the stainless steel container. The mixture is then subject to colloidal milling. In some embodiments, the resulting particles are further processed through multi-stage milling, for example using zirconium oxide milling media of decreasing size.

Once the milling is complete, a homogeneous composition of fine particulate, biologically activated ion-exchange polymer salt particles is obtained (often after separation of the particles from the non-solvent liquid by evaporation, and from the milling media by mechanical separation, e.g., sieving). This activated, fine particulate ion-exchange polymer salt product has been shown to be cosmetically acceptable, with excellent biological activity potential (e.g., antimicrobial character) over a broad range of weight % loadings of the starting ion-exchange polymer salt with biologically active substitute counter-ions.

In more detailed examples of high energy milling, porous activated ion-exchange polymer salt particles are size-reduction milled by high energy milling with milling media and a non-solvent liquid (typically in a sealable milling container, but alternatively in high-throughput, pass-through milling apparatus). In some embodiments, the sealable milling container has a liner made of suitable material of comparable hardness as the milling media, for example a ceramic lining adapted for ceramic milling media. The milling media, for example zirconium, beads may be in any suitable size from about 0.1 mm to about 10 mm in diameter, about 0.5 to about 5 mm, about 1 to about 5 mm, about 4 to about 5 mm, about 0.5, to about 1 mm. The non-solvent liquid may be any low volatility liquid inert to the resin and the biologically active agent. In certain embodiments, the non-solvent liquid is an organic non-solvent such as an alkane. Exemplary alkanes include heptane, isooctane, and octane, among other known alkanes with suitably low boiling points.

The non-solvent liquid fills the voids within and between the porous ion-exchange polymer salt particles (and interstices between these particles and milling media) and functions to oppose compression of particle structures (particularly walls and partitions of voids, pores and channels) from impact, shear, friction, pressure and other mechanical forces during the milling process. To effectuate this efficient and uniform particle fracturing, the milling container may be filled to ⅓ of its volume with the porous activated ion-exchange resin particles, and to roughly a remaining ⅔ of its volume with the milling media. This leaves approximately ⅓ of the container volume available as interstitial space between milling media units, within the interstices between polymer salt particles and media, and within porous depressions, voids and channels of the activated ion-exchange polymer salt material. This approximately ⅓ remaining volume within the milling chamber of the container is filled with the non-solvent liquid to fill the interstitial and void spaces and channels as described.

During milling the combination of high energy milling forces and the milling media rupture the walls of void spaces in the resin occupied by the non-solvent. The solvent renders the particles non-compressible to impact, shear and other forces during the milling—resulting in highly efficient and uniform fracturing and rupture of the particles to a final milled average size and size variation as described. This novel milling process for biologically activated ion-exchange polymer salts is additionally aided by controlling milling temperature. Here, milling apparatus and methods are selected which provide for a controlled milling temperature in a range from about 70 to about 95° C. often between about 75 to about 90° C., and in exemplary embodiments from about 82 to about 87° C. or approximately 85° C. In certain embodiments of the invention, artificial heating of the milling chamber is not required, rather heat generated by high energy milling friction passively controls the milling temperature (adjustable by controlling milling speed, media composition and size, non-solvent liquid selection, etc.) within a selected range of from about 70 to about 90 degrees, about 75 to about 90 degrees, or other adjustable milling temperature ranges, for example at or about a target milling temperature of 85 degrees or 90° C.

Using these and other exemplary high energy milling methods, apparatus and formulae, fine particulate ion-exchange polymer salt materials for use within the invention can be routinely produced with desired particle diameters between about 10 nm to 100 μm, about 30 nm to about 50 μm, about 100 nm to about 10 μm, about 200 nm to about 1 μm, or about 400 nm to about 600 nm, for example. In some embodiments, the material is milled to a uniform particle size of about 200 nm, 400 nm, 600 nm, or 800 nm. In other exemplary embodiments, an average particle diameter of 500 nm is provided, with very low particle size variation as described. Each of the specified, distinct particle size values described here corresponds to novel biological activity potential for the fine particulate ion-exchange polymer salt materials, and for polymer composites incorporating these novel materials. This degree of particle size selectability and uniformity is not obtainable with other milling methods, such as dry milling methods—in part due to the tensile strength, elasticity and compressibility of ion-exchange polymer salts under ordinary milling conditions.

In certain embodiments, targeted milling size distributions possess larger standard deviations for a first reduction, e.g., from particles as large as 5000 micron (with ±5-10 microns as an exemplary standard deviation, in other embodiments between ±2-7 micron, or between ±1-3 microns or lower) while following a second reduction step final particle size may average 500 nm average diameter with a standard deviation of approximately ±0.75 microns (in other embodiments lesser than or equal to ±0.50 microns, or lesser than or equal to ±0.25 microns).

Once the fine particulate activated ion-exchange polymer salt particles are milled to a desired size, they are isolated if required (e.g., separated from milling media and non-solvent liquid). In exemplary embodiments, ceramic or other milling media may be removed by mechanical separation, such as sieving. Non-solvent liquids may be removed by any means generally used, most often involving evaporation. In some embodiments, due to the volatile nature of some non-solvents, this liquid is removed by controlled evaporation (to prevent harmful release of evaporated solvent into the environment, and to prevent “bumping” of the fine particulate ion-exchange polymer salt material during drying. Controlled evaporation may be conducted in a static or vacuum oven depending on the volatility of the solvent.

In some embodiments high energy milling is a multi-stage process, for example where milling is repeated 2 or more times with successively smaller sized milling media to achieve a desired particle size. The same or different grinding media and the same or different non-solvent liquids may be used in successive milling stages as required to achieve appropriately sized fine particulate ion-exchange polymer salt products as described.

In certain embodiments of the invention gel-based resins are selected for constructing biologically activated polymer salts. These discrete resins, often used for conventional water treatment applications, have inherently greater loading capacity and regeneration efficiency. Macroporous (aka macroreticular) resins are generally preferred in more aggressive applications where their highly cross-linked structure is an advantage. (Examples: applications subjected to large osmonic shock, feedwater with elevated chlorine content, higher temperature applications). The milling procedures and materials described here can be applied to both gel-types, macroreticular and macroporous. In certain examples, a conventional gel type, styrenic ion exchanger is built on a matrix prepared by co-polymerizing styrene and DVB. In these systems, porosity is inversely related to the DVB cross-linking. Gel resins exhibit microporosity with pore volumes typically up to 10 or 15 Angstroms. Macroporous (macroreticular) ion exchange resins have pores of a considerably larger size than those of the gel type resins with pore diameters up to several hundred øngstroms. Their surface area may reach 500 m2/g or higher. Macroporous polymers are generally highly cross-linked and therefore exhibit little volume change (swelling). Because of the high cross-linkage in the matrix, the apparent oxidation stability of macroporous resins is improved. However, at similar crosslinkages, macroporous resins have greater exposure to potential oxidants than gel resins due to their greater porosity and surface area

Functionalized anion- or cation-exchange materials are reversibly or non-reversibly associated with a selected, anionic or cationic biologically active agent by various operable methods and formulae for ion-exchange chemistry. Typically, the selected ion-exchange polymer (functionalized and associated with initial counter-ion, e.g., Na+ for cation-exchange examples, as described) is placed in an aqueous medium in a particulate form and combined with the replacement, biologically active counter-ion (typically added to the aqueous medium as a salt form of the biologically active agent (e.g., silver acetate). Combining the particles of ion-exchange polymer material with a salt comprising an antimicrobial cation, for example, in an aqueous medium will mediate salt-exchange of the antimicrobial cation for the initial counter-cation present on the ion-exchange polymer—to yield an antimicrobially activated polymer salt derivative (having the antimicrobial cation ionically associated with the polymer). Typically, these salt-exchange processes will render the newly-associated, biologically active counter-ion effectively insoluble in water (i.e., the active counter-ion will not freely dissociate in distilled water).

This insolubility or non-dissociability can be controlled to allow for partial solubility or dissociability of the active counter-ion from the activated ion-exchange polymer salt, for example by using weaker ion-exchange materials, multivalent active counter-ion agents, and other methods. Thus, in certain embodiments of the invention, the biologically active counter-ion agent may be partially soluble in ionic aqueous media, or may be completely, reversibly associated with the ion-exchange polymer such that it is insoluble in distilled water and other non-ionic media, but rendered freely soluble in ionic media such as saline and physiological fluids. In this manner the biologically activated polymer salts and related composites of the invention can function in multiple activity modalities. In primary activity modality, the activated polymer salts and composites exert their biological effects mostly as “surface activity”, where the biologically active ionic agent functions primarily at a surface of the polymer salt or composite, without appreciable (e.g., greater than 5%) dissociation (typically solubilization) of the active ionic agent from the surface.

In an alternative or combined modality, the activated polymer salts and composites can also exert “non-surface” biological effects as drug delivery materials or devices, wherein in addition to “surface activity” the biologically active ionic agent is also “reversibly-associated” with functional groups on the ion-exchange polymer salt materials in the composites. They are therefore ionically dissociable from the composite surface under certain conditions, and can be released in a soluble form following exposure to, e.g., ionic aqueous media including physiological fluids. In these aspects of the invention, polymer composites incorporating activated ion-exchange polymer salts function as drug and active agent delivery materials and devices—i.e., to deliver dissociated, biologically active ionic agents to tissue and compartments adjacent to or distant from the polymer salt/polymer composite surface.

Generally, the surface area of the device is a significant factor in delivery (e.g. foams yield high surface areas, versus a lower surface area, textured or solid composite material). Surface area of different constructs can be controlled, for example by material choice, and by fabrication and molding techniques (such as spraying, coating, blowing, molding and extrusion techniques that include co-extrusion). In certain embodiments it is important to restrict contact of an activated (e.g., silicone) composite material with a surface (e.g., an inner lumen) or portion of a device the composite is being attached, layered or molded to. The hydrophilicity of the polymer matrix may also plays a role in the surface release characteristics of materials and devices of the invention.

The dissociation constants of the fine particulate activated ion-exchange polymer salt particles can be compared to the counterpart simple salts, particularly for silver given the known (low) solubility for silver salts. For example, silver sulfate (Ag₂ SO₄) possesses a solubility constant (Kₛₚ) of 1.2×10⁻⁵, silver chloride (AgCl) possesses a Kₛₚ of 1.77×10⁻¹⁰, and silver phosphate possesses a Kₛₚ of 1.8×10⁻¹⁸. As such, strong cation-exchange fine particulate activated ion-exchange polymer salt particles modified to include silver will certainly possess a Kₛₚ<1.2×10⁻⁵. With the replacement of silver by a cationic replacement ion and its simultaneous release and pairing with an anion (chloride, phosphate etc.), dissociability of the product salt is important. A surprising advantage of the instant invention is that replacement of the departing ion (e.g., silver) from the fine particulate activated ion-exchange polymer salt particles remedies the general concern of void spaces that would otherwise form when soluble components dissolve from conventional polymers and coatings.

To control dissociability and/or drug delivery kinetics of biologically active ionic agents from activated polymer salt materials and related composites, more and less hydrophilic and hydrophobic polymer matrices can be used. Distinct performance characteristics provide for sensitive construction of activated polymer salts having a full range of activity modalities, from purely surface activity to increasing levels of reversible or dissociable loading (including adjustable release and solubilization of initially bound, biologically active counter-ion agent, as can optionally be triggered by contact with physiological/ionic fluids). For preparing these adjustable release, activated polymer salt constructs having different activity modalities and dissociation potential/kinetics, a wide range of useful ion-exchange polymer salts are provided.

In more detailed embodiments of the invention, the fine particulate activated ion-exchange polymer salt materials thus produced are useful in a wide variety of biomedical methods, compositions, materials, polymer composites, and devices including devices where a hydrophilic matrix (carrier) is employed. Such applications include hydrophilic coatings on the surfaces of medical devices such as catheters (tubing) and hydrophilic carriers such as in foams, sponges, and sheet-stock materials that can be used in wound healing (vacuum-assisted closure), wound dressings, vaginal sponges and the like.

In other embodiments of the invention, the fine particulate activated ion-exchange polymer salt materials may be incorporated into bitumen, asphalt, or tar for the purposes of coating substrates. One such application may include coating of the inside of duct work in order to minimize pathogens in environments that require good adhesion and chemical stability for example. In addition, the incorporation of the fine particulate activated ion-exchange polymer salt materials into cellulose (paper) and/or gypsum board material can allow for the fabrication of gypsum wall board with antimicrobial properties as for example to minimize or prevent the growth of fungi. This may be done with the use of a copper salt of the fine particulate activated ion-exchange polymer salt material or a more active (organic) cationic fungicide derivative.

Certain embodiments of the invention employ fine particulate activated ion-exchange polymer salt materials absent a polymeric binder, or with only an aqueous-based carrier that can be employed in order to disperse the particulate materials. For example, the fine particulate activated ion-exchange polymer salt materials may be used in farming to deliver fungicides, nutrients, or insecticides for example. One such example is an azide derivative of an anionic exchange material. Azide is used often in pest control. In this instance, the fine particulate activated ion-exchange polymer salt materials may be encapsulated into a starch carrier thus allowing for safer and more facile spreading of the particulates.

In other embodiments of the invention, azide derivatives of fine particulate activated ion-exchange polymer salt materials are employed in the fabrication of airbags and are safer to handle and will perform better than the conventional airbag material sodium azide. Similar products of the invention also possess preservative activity and can also be used in the fabrication of detonators and other explosives (particularly employing high surface area constructs). For these applications crosslinked materials are employed wherein the crosslinker is enzymatically degradable, for example a divinyl adipate. Similar to azide, fulminate derivatives of fine particulate activated ion-exchange polymer salt materials may also be employed as a detonator composition.

In certain embodiments of the invention, cyano (cyanide) derivatives of fine particulate activated ion-exchange polymer salt materials are employed as a means of forming cyanide. These derivatives can be used as a means of dispersing (weaponizing) hydrogen cyanide.

Fine particulate activated ion-exchange polymer salt materials of the invention are also useful for environmental recovery of soluble metallic and organic contaminants, particularly in fresh water. These compositions and methods employ high surface area foam materials containing dispersed fine particulate activated ion-exchange polymer salt materials. The subject foams, pads and/or sponges can be constructed for capture of selected metal(s), for example lead (wherein Pb (II) is captured by a weak cation-exchange material integrated in a moderately hydrophilic material coated onto a three-dimensional lightweight substrate such as a polymer foam, metal substrate such as a fence-like substrate, tubes with pores, or a carbon construct, for example). These constructs are placed into an environment at risk of contamination and removed and replaced as needed.

In certain embodiments of the invention, fine particulate activated ion-exchange polymer salt materials are combined with other polymer materials to produce biologically activated solidified polymer composites. The fine particulate ion-exchange polymer salt is generally admixed in effective amounts with precursors of a thermoset or thermoplastic or photocuring polymer, to form fluid or semi-solid antimicrobial polymer composite mixtures. The mixtures can be solidified using a wide range of polymer manufacturing methods and conditions and in a diverse array of composite mixtures and final hardened composite forms (e.g., solid cast or molded articles or components, extruded, spun into fiber, or blown into solid or cellular set polymer (film) materials, laminates, coatings, paints, and the like. The biologically activated solid polymer composites are formed by solidifying, drying or curing the polymer precursors admixed with the fine particulate biologically activated ion-exchange polymer salt material. In some embodiments, the fine particulate polymer salt material is distributed throughout the resulting, activated polymer composite for example as in a polypropylene suture as fabricated by drawing, extrusion, or spinning and incorporating an evenly distributed composition of the fine particulate polymer salt material(s). In such an embodiment, the fine particulate polymer salt material may be modified to include one or more of tobramycin, minocycline, or silver or mixtures of the individual fine particulate polymer salt materials may be used for example in order to render the suture material antimicrobial. In another example embodiment, a polypropylene composite material, for example to include silver (I), copper (II), zinc (II), benzalkonium, sodium, alone or in combinations thereof can be spun into a non-woven (fabric) composition and the non-woven material composition used in the fabrication of air filters, carpet, furniture, medical textiles, and geotextiles. One example application is for use as a (diagnostic) substrate when formulated to include IRP69-Na. Such a substrate can be placed below ground, allowed to dwell for some period of time and subsequently harvested (removed from the ground) and the fabric analyzed for metal uptake (e.g. Cu (II), Fe (II), As (III, V for example) with the aid of atomic absorption (AA) or inductively coupled plasma (ICP) spectroscopy. In yet another embodiment a hernia repair patch may be constructed using similar means yet with an IRP69 derivative functionalized with tobramycin for example. In other embodiments, the fine particulate polymer salt material is unevenly distributed within the final solid composite. This can be achieved, for example, by mixing the fine particulate activated polymer salt material only with specific parts or layers of a composite mixture prior to hardening. In this manner, setting of the ion-exchange polymer salt in the hardened polymer composite will determine its localization in a predetermined functional spatial distribution within the hardened composite, for example by concentrating the polymer salt particles at upper, outer, luminal, or other defined sites, surfaces, layers or areas within a solid composite form or structure. Methods available for site-specific location of the particles includes coating these areas using dipping, spraying, or painting (e.g., acrylic, latex, enamel or epoxy-based paints) onto surfaces, direct application (including affixing by direct adhesion, attachment means, or gluing) of fluid, semi-fluid or solid composites onto surfaces, including laminating or molding over metal, wood, polymer or other types of substrates, co-extrusion, etc. The various methods and forms of composite application provided here include, for example, for example, coating an exterior surface or interior surface of a medical device, tool, apparatus, appliance, furnishing (or medical facility wall, floor, ceiling or fixture) or medical or surgical material (e.g., an outer surface or lumen of a medical tube, endoscopic device or catheter). Because extrusion is a continuous process, certain surface coatings comprising a composite of the invention will be formed continuous along a surface of a device, device component, functional surface, product or finished material. In the event that more than one modified fine particulate activated ion-exchange polymer salt material is desired in a single construct, more than one extrusion feed may be used.

In yet another example, inkjet technology may be employed to deposit an array of various coatings or paints comprising a fine particulate activated ion-exchange polymer salt material integrated in a suitable polymer or polymer mixture, to form a paintable liquid polymer composite mixture that can be sprayed, painted or otherwise coated or laminated onto a surface (e.g., of a medical device, surgical device, tool or furnishing, or diagnostic or environmental testing tool (e.g., a probe to test environmental contamination such as heavy metals). In other examples, small molecule probes may be isolated onto particles and further isolated onto an array to probe for viruses or bacteria, or to analyze/detect genetic markers of a pathogen, parasite, food-borne infectious or toxic microbe, or any of a wide range of other clinical diagnostic, environmental or infectious disease variables.

The thermoset or thermoplastic or photocuring polymers used to form solid biologically activated polymer composites herein can be selected from a broad assemblage of useful polymers, for example polysiloxane, polyalkylene, polyamide, epoxy, polycarbonate, polyester, vinyl, acrylic, and polyurethane polymers, and combinations thereof. In certain embodiments, the thermoset or thermoplastic or photocuring polymer mixed with the fine particulate activated ion-exchange polymer salt material (comprising the “polymer composite mixture”) is cast, sprayed, formed, spun, blown or extruded into a desired shape or article prior to solidifying. The polymer composite mixture may be solidified by any means generally used, for example by drying or curing under normal conditions (e.g., at room temperature in air). In certain embodiments the polymer composite mixture may be cooled during hardening process, while in other embodiments the polymer composite mixture is cured using heat. In additional embodiments, the second, thermoset or thermoplastic or photocuring polymer precursors are provided in the form of a polymer lacquer, the lacquer comprising a solvent, the solidifying step comprising evaporating the solvent from the polymer lacquer to form the solid biologically active polymer composite. The resulting solid biologically active composite may contain a selected amount or weight ratio of the activated ion-exchange polymer salt material, as described, to optimize the composites for specific uses and concentrations (or effective dosage levels) of incorporated biologically active ionic agent to mediate specific biological activities and/or therapeutic effects.

In certain embodiments relating to construction of polymer composites, the thermoset or thermoplastic or photocuring polymer precursors are non-vulcanized silicone rubber precursors. These precursors combine to form a highly adhesive silicone gel or liquid. The silicone gel or liquid is cured after addition of a selected amount or ratio of the fine particulate activated ion-exchange polymer salt, often at an elevated temperature of about 150° C. (typically for a curing period of about 5 to 10 minutes). In certain embodiments where the fine particulate ion-exchange polymer salt incorporates an oligodynamic metal, such as silver, as the activating ionic agent, curing of the silicone polymer results in discoloration, marked by darkening (often with a reddish tint) of the hardened biologically activated polymer composite.

Yet another useful and unexpected discovery of the invention is that certain activated polymer composites may be further processed to reverse normal curing discoloration, to yield a re-lightened final solid polymer composite. The further processed, lightened polymer composite is more advantageous for medical and other uses, from both a basic cosmetic appeal perspective (lighter polymer materials appear more hygienic), and from an actual hygiene and safety perspective (because the lighter color allows for better visualization of soiling agents and contaminants, including possible toxic, pathogenic or corrosive contaminants).

Reversal of discoloration from normal curing of activated polymer composites of the invention (particularly those containing silver and other metallic ions) can be achieved by employing the novel polymer composite mixtures provided herein, and by subjecting these discrete polymer composite mixtures to a modified curing regimen. The latter discovery focuses on extended curing times and/or elevated curing temperatures, which alone or in combination (typically in the presence of oxygen) yields a surprising reversal of color darkening observed following conventional curing procedures.

Discoloration reversal can be achieved for example by extending curing times beyond conventional curing times (e.g., 5-10 minutes for silicone). Thus in certain embodiments curing times may be extended for an additional 10-30 minutes, one-three hours, or longer depending upon composition of the polymer composite. In other embodiments initial and/or extended curing may be conducted at a higher temperature than conventional curing, for example at temperatures greater than 150° C., greater than 175° C., up to 200° C. or higher. In exemplary protocols, normal curing is conducted at 150 degrees for 5-10 minutes, and extended curing is carried out for an additional time period until a desired extent of discoloration reversal is observed. These curing changes, in various protocols following the teachings herein, yield novel biologically activated polymer composites having desirable, lightened color properties for medical and other uses.

Certain activated polymer composites of the invention are made using multiple different polymer precursors, for example a mixture of polymer precursors of polyalkylene, polysiloxane, polyamide, epoxy, polycarbonate, polyester, polyol, polyarylene, vinyl polymer, acrylic polymer (polyacrylonitrile, polyacrylate), asphalt, bitumen, polysaccharide, cellulosic, and/or polyurethane. The polymer precursors for making the activated polymer composites can include one, two or more types of precursors selected from silicone rubber, methacrylic acid, polypropylene oxide, polyethylene oxide, polyvinyl alcohol, polyurethane, hydrocolloid, a polyester, a polycarbonate, a vinyl polymer (PVC, PVA, PVAc, Polyvinylidene chloride, polyisoprene, styrenic polymers including polystyrene, styrene-isobutylene-styrene triblock copolymer (SIBS), acrylonitrile-butadiene-styrene copolymer ABS, styrene-butadiene-styrene copolymer (SBS), hydrogenated vinyl polymers including hydrogenated SBS, e.g. styrene-ethylene-butylene-styrene copolymer (SEBS), and polyalkylenes such as polyethylene and polypropylene, a polyamide, an epoxy, a phenolic resin, a polyurea, an acrylic, a cellulosic, a fluoropolymer, or a biopolymer such as collagen, hyaluronic acid, gelatin, a hydrogel polymer, and/or an alginate, among other polymer types. The precursors may include like or different monomers including monomers of block, graft and statistical copolymers, asphalt, bitumen, and/or blends of various polymers.

Solid polymer composites of the invention can include a plurality of polymer chains from at least one polymer type forming a solid polymer matrix. The same polymer precursors can be used to form different types of solid or semi-solid polymer matrices. For example a silicone rubber polymer solid or semi-solid matrix can comprise a silicone rubber adhesive, a tacky silicone gel, a liquid silicone rubber, or a high consistency silicone rubber. The solid polymer matrix may be an elastomer, which when in solid form employed for making durable materials and products will often have a hardness (durometer) in the range of 10 shore A to 90 shore D. In some embodiments, the hardness of biologically activated solid polymer composites and manufactures may be between 15 shore A and about 65 shore D. Other “engineering polymers” may also be employed. These include acrylics, polycarbonate, poly(ether-ether-ketone) (PEEK), acrylonitrile-butadiene-styrene (ABS) polymers, as well as other materials amenable to thermal processing or processing into lacquers for coating processes.

Production of certain biologically activated solid polymer composites of the invention is schematically depicted in manufacturing Scheme 4, where R is a group containing carbon and n is greater than 1.

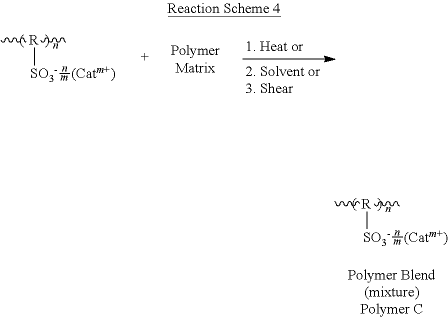

The polymer matrix (precursors) may be a polymeric composition that includes one or more useful polymer precursor types, for example from the group silicone rubber, polyurethane, a polyester, a polycarbonate, a vinyl polymer (PVC, PVA, PVAc, Polyvinylidene chloride, polyisoprene, SIBS, ABS, SBS, polystyrene, hydrogenated vinyl polymers, e.g. SEBS), a polyalkylene such as polyethylene, a polyamide, an epoxy, an acrylic, a cellulosic, a fluoropolymer, or a biopolymer such as collagen, hyaluronic acid, gelatin, a hydrogel polymer, and/or an alginate.

In some embodiments, the polymer precursors comprising the polymer matrix may be provided as one or more polymer precursors in a substantially unsolidified (fluid or semi-solid) state. Prior to solidifying the polymer composite, the precursors are blended with biologically activated ion-exchange polymer salt particles to form a polymer composite mixture. This mixture is then solidified to form activated solid polymeric composites, and related biomaterials and products.

In various embodiments, activated polymer composites are made with any of a diverse array of polymer precursors classified as thermoplastic, thermoset, elastomer, and/or rigid polymer precursors. Exemplary polymeric precursors include, but are not limited to, one or more of polyalkylene, polysiloxane, polyamide, epoxy, polycarbonate, polyester, polyol, polyarylene, vinyl polymer, acrylic polymer (polyacrylonitrile, polyacrylate, methylmethacrylate), asphalt, bitumen, polysaccharide, cellulosic, and/or polyurethane. Exemplary polymer precursors comprise nonvulcanized silicone rubber precursors.

In other embodiments, solid (hard materials) such as polycarbonates, and epoxies can be combined with fine particulate biologically activated polymer salts and these types of polymer composite mixtures can be formed and solidified to provide harder materials having smoother, harder, more impact resistant and defect-free surfaces than other polymer composites herein. Exemplary activated polymer composites produced according to the teachings herein are listed in Tables 2a-2c below, for illustrative purposes.

TABLE 2A

| | Matrix | Active Agent - Singular formulations | | | | | | | | | | | | | |
|---|---|---|---|---|---|---|---|---|---|---|---|---|---|---|---|
| Resin | Material | Ag | BA | CP | Dox | Rif | Min | CHX | Oct | Zn | Fe(II) | Cu | Ag/Cu | Ag/Zn | BA/Ag |
| IRP69 | Silicone | x | x | x | x | x | x | x | x | x | x | x | x | x | x |
| | Polyurethane | x | x | x | x | x | x | x | x | x | x | x | x | x | x |
| | Epoxy | x | x | x | x | x | x | x | x | x | x | x | x | x | x |
| | Acrylic | x | x | x | x | x | x | x | x | x | x | x | x | x | x |
| IRP64 | Silicone | x | x | x | x | x | x | x | x | x | x | x | x | x | x |
| | Polyurethane | x | x | x | x | x | x | x | x | x | x | x | x | x | x |
| | Epoxy | x | x | x | x | x | x | x | x | x | x | x | x | x | x |
| | Acrylic | x | x | x | x | x | x | x | x | x | x | x | x | x | x |

TABLE 2B

| | | Binary Formulations (Individual Singular Additives) | | | | | | |
|---|---|---|---|---|---|---|---|---|
| | Matrix | Ag & | Ag & | Ag & | Ag & | Rif & | Dox & | Ag & |
| Resin | Material | BA | CP | Zn | Zn | Min | Rif | CHX |
| IRP69 | Silicone | x | x | x | x | x | x | x |
| | Poly- | x | x | x | x | x | x | x |
| | urethane | | | | | | | |
| | Epoxy | x | x | x | x | x | x | x |
| | Acrylic | x | x | x | x | x | x | x |
| IRP64 | Silicone | x | x | x | x | x | x | x |
| | Poly- | x | x | x | x | x | x | x |
| | urethane | | | | | | | |
| | Epoxy | x | x | x | x | x | x | x |
| | Acrylic | x | x | x | x | x | x | x |

TABLE 2C

| Active Agents | | | Polymer | Company | Product Name |
|---|---|---|---|---|---|
| Ag = Silver | Rif = Rifampicin | Zn = Zinc | Silicone | Nusil | 4950 |
| BA = Banzalkonium | Min = Minocycline | Fe = Iron | | Dow Corning | Q7-4750HCR |
| CP = Cetylpyridinium | CHX = Chlorhexidine | Cu = Copper | Polyurethane | Lubrizol | MG-8020 |
| Dox = doxycycline | Oct = Octenidine | | | Lubrizol | TG-500 |
| | | | | Lubrizol | SP-80A-150 |

| | | | |
|---|---|---|---|
| Ion Exchange Resins | Epoxy | EPO-TECH | 301 |
| IRP64 = Poly(methacrylic acid-co-divinylbenzene) | Acrylic | SCIGRIP | 40 |
| IRP69 = Sulfonated Poly(styrene-co-divinylbenzene) | | | |

Primary products of the invention (biologically activated ion-exchange polymer salts) can be combined with a variety of thermoset or thermoplastic or photocuring polymer precursors to make solid composites having a range of biological surface activities (and optionally, non-surface drug delivery activity) and a commensurate array of applications and methods of use. Surface activation of the inventive polymer composites (i.e., specific biological activity or activity potential, exhibited at an exposed surface of the polymer composite) can vary depending on the type and identity biologically active ionic agent incorporated in the ion-exchange polymer salt, as well as on the amount and distribution of the activated polymer salt within the hardened polymer composite.

Among the powerful discoveries here, biologically activated solid polymer composites can incorporate varying amounts of the activated ion-exchange polymer salt material to yield predetermined or “metered” activity potential at the solid polymer composite surface. Varying the amount or distribution of activated ion-exchange polymer salt can increase or decrease the surface activity of the finished polymer composite, by increasing or decreasing a surface concentration (e.g., by weight or by surface area) and activity of the biologically active ionic agent associated within the activated polymer salt. This ability to adjust or “meter” surface activity of polymer composites is readily achieved according to multiple teachings herein. In one example, this is achieved by adjusting “loading” of the ion-exchange polymer as described (e.g., by increasing or decreasing a percentage of biologically active counter-ion-exchange for initial counter-ion within the ion-exchange polymer—expressed for example as a percent of actual exchange (with activating counter-ion) of real or theoretic maximum ion-exchange potential, or in another example as, e.g., weight of silver or other active counter-ion loaded per total dry weight of ion-exchange material).

In another (alternative or complementary) method for controlling surface biological activity of activated polymer composites of the invention, the instant disclosure provides for variable or metered “dosing” of polymer composites by combining different amounts of fine particulate, biologically activated ion-exchange polymer with thermoset or thermoplastic or photocuring precursors to form the activated composites. Surface activity potential (and in related embodiments dissociation and drug delivery kinetics) are therefore adjustable across a wide range of selectable values, simply by adjusting a weight percentage of activated polymer salt to thermoset or thermoplastic or photocuring polymer precursors, as described. A selected weight ratio of 10-20% of activated, fine particulate polymer salt combined with silicone precursors to form an activated composite, for example, will yield approximately twice the surface activity potential (and optionally twice the dissociation or drug delivery kinetic value) of a like composite formed using only 5-10% by weight of the activated fine particulate polymer salt.

In certain embodiments of the invention, the biologically activated polymer salts and polymer composites are useful to prevent attachment, colonization and/or survival of microbes (e.g., bacteria, viruses and/or fungi) or other pathogens or parasites transmissible by surface contamination on a fomite or other targeted surface. To the extent colonization of a surface bearing an activated, antimicrobial polymer salt or polymer composite of the invention is subject to “contamination” by a subject microbe or other pathogen, the activated polymer salt or composite functions distinctly by reducing or preventing secondary transmission of viable pathogens to a vulnerable living subject, for example a veterinary or human patient in a clinical or home medical care environment. By possessing activated, antimicrobial (e.g., bactericidal or bacteriostatic) surface activity, the invention either prevents or limits contamination, or reduces bacterial growth or viability on contaminated surfaces, such that when these surfaces are secondarily brought into contact with a living subject the rate of transmission or “infection” from an activated polymer surface to the subject (e.g., compared to a surface made of the same material and exposed to the same experimental contamination inoculum, not activated by incorporation of a biologically activated polymer salt carrying the biologically active ionic (antimicrobial) agent).

These distinctly potent antimicrobial and other antipathogenic activities are readily demonstrated using conventional assays. For example, antimicrobially activated polymer composites of the invention incorporating an ionic antibacterial agent (e.g., silver, or an ionic antibiotic) will exhibit a much reduced risk of effective contamination compared to the same material that is non-activated. In side-by-side tests (e.g., of the same silicone polymer, with and without incorporation of an antibacterially activated polymer salt resin as described here, where the test and control polymers are subject to the same inoculum of contaminating test bacteria), re-plating the contaminating bacteria (e.g., by swiping an equal area of test and control, “contaminated” surface across an agar culture medium) to a secondary test surface demonstrates great efficacy of the materials of the invention in preventing and controlling microbial contamination. Comparable efficacy is obtained using related embodiments of the invention incorporating fungicidal and fungistatic ionic agents, antiviral ionic agents, and anti-parasitic ionic agents (while some of these agents will have efficacy against multiple pathogen groups).

To quantify these distinct surface properties, the invention as tested using antimicrobially activated polymer composites effectively prevents or reduces microbial contamination and transfer up to 100% in side-by-side assays (e.g., as demonstrated by Kirby-Bauer disk diffusion assays described below). In more detailed aspects, the biologically activated polymer salts and polymer composites of the invention prevent or reduce persistent microbial contamination (and, commensurately reduce microbial transfer potential) by at least 20-30%, 30-50%, 50-75%, or 75-90%, up to as much as 90-95%, or 98% or greater compared to persistent contamination and transfer potential observed using control materials. In various assays demonstrating these novel activities, microbial survival, viability and/or growth potential is reduced within these value ranges after inoculating test and control surfaces, waiting for a suitable post-inoculation period (to allow for activity potential of the test and control samples to be expressed, e.g., to permit bactericidal and bacteriostatic activity to take place), followed by “transfer plating” or “transfer culturing” to test survival and viability/transferability of microbial contaminants from the test and control materials/surfaces. The latter determination is made, for example, by directly contacting contaminated test and control surfaces to a “transfer” culture plate or liquid culture medium, or using lavage to transfer any intact and/or viable microorganisms from test and control surfaces, then detecting presence, numbers, or viable contagious units (e.g., colony forming units, or CFUs) in the transfer growth plate or medium.

According to these methods, the activated polymer salts and composites of the invention exhibit extraordinarily high levels of surface decontamination activity (e.g., bactericidal and/or bacteriostatic surface activity). This potent activity manifests within as little as 1-10 minutes after inoculation/contamination of these unique biomaterials. Within a half hour after surface contamination, or in some instances after from one hour to three hours, full expression of maximal surface decontamination activity is observed for many antimicrobially activated polymer salts and polymer composites of the invention. In many instances this amounts to an effective total surface decontamination, where consistently no viable microorganisms remain viable or transferable from a contaminated surface after a post-inoculation activity expression period. These observed results are truly remarkable in comparison to contamination and transfer data observed from similarly treated control biomaterials (i.e., comparable ion-exchange polymer salt materials not activated by association with biologically active counter-ions, or comparable polymer composites incorporating ion-exchange polymer salt materials not activated with biologically active counter-ion).

In exemplary embodiments, microbial survival and/or transfer potential (e.g., expressed in terms of microbial numbers or growth observed after transfer plating from the contaminated surface/material) from contaminated test samples (of either the fine particulate ion-exchange polymer salt, or polymer composites made therewith) is less than 50% of microbial survival and/or transfer potential observed from control samples. In other embodiments, the microbial survival and/or transfer potential for test materials is less than 25%, 15%, 5% or 1% of the microbial survival and/or transfer potential observed from control materials. These and even higher levels of decontamination and transfer risk reduction are achieved for various microbial pathogens, including different forms of pathogenic bacteria, as well as pathogenic fungi and other microbial pathogens. In exemplary, antibacterial materials and composites, the level of bacterial control and decontamination mediated by polymer salts and composites of the invention confers at least a 50-75% reduction, often a 75%-95% reduction, up to a 95%-100% reduction and/or prevention of persistent contamination and/or transfer risk.

Results for post-contamination transfer potential, or infection risk, are even more surprising and beneficial using the antimicrobially activated materials and composites of the invention. The subject materials and composites have such novel and powerful surface antimicrobial efficacy, they can substantially eliminate surface-to-living subject transfer of viable pathogens targeted by their surface-loaded ionic antimicrobial agents. For ease of description, retransmission potential (e.g., as measured by ability to transfer viable colony forming units of a targeted bacterium from a contaminated surface following a “decontamination period” (of, e.g., 10-30 minutes, 1-3 hours, or longer) is reduced by at least 75-95%, often greater than 95%, and reproducibly at levels of up 98-100% compared to similarly contaminated controls of like polymer materials not antimicrobially activated according to the invention.

The profound antimicrobial surface activity exhibited by novel polymer composites of the invention renders these materials widely effective against a large host of the most serious bacterial contaminants found in institutional care settings and environments. Effective materials and products are provided against the most refractory, costly and dangerous sources of infection found in medical and veterinary care hospitals, assisted living facilities, penal housing institutions, food processing and packaging facilities, and HVAC and other environmental control systems, among other environments. Targeted microbes subject to reduction of surface contamination, and elimination of surface-to-live subject transfer risk, as described, include, for example, Staphylococcus, Pseudomonas, Escherichia coli, Klebsiella pneumoniae, Legionella, Mycobacteria, Streptococcus, Acinetobacter, Haemophilus, and Enterococcus, Aspergillis, and Listeria.

Yet additional advantages afforded by the instant invention include a novel utility and efficacy against infectious targets resistant to many drugs, such as MRSA (methicillin-resistant Staphylococcus aureus), resistant Streptococcus strains, and resistant airborne pathogens such as Mycobacterium tuberculosis and Legionella pneumophila. Using the embodiments of the invention described herein, resistant organisms may be addressed using compositions of distinctly modified fine particulate activated ion-exchange polymer salt materials in combination. In one embodiment a silver-modified fine particulate activated ion-exchange polymer salt material is combined with a chlorhexidine-modified a fine particulate activated ion-exchange polymer salt material and the mixture is added to a polymer composition to produce a binary delivery system. Because each of the antiseptics kill bacteria using unique mechanisms, the likelihood of selecting for resistant strains is greatly diminished.

Yet additional advantages afforded by the instant invention include a novel utility and efficacy against infectious fungal diseases such as onychomycosis (fungal infection of the toe- and fingernails), tinea pedis, jock itch, ring worm, or cutaneous candidiasis. Antifungal agents can include copper (II), polyenes, imidazoles, triazoles, thiazoles, allylamines, echinocandins (caspofungin), flucytosine, and crystal violet. Generally, the aforementioned functional compound types may be used topically more effectively than by oral delivery. For example, for the treatment of onychomycosis, where Trichophylon rubrum is the most common dermatophyte involved in onychomycosis. Other dermatophytes that may be involved are Trichophyton interdigitale, Epidermophyton floccosum, T. violaceum, Microsporum gypseum, Trichophyton tonsurans, and Trichophyton soudanense. Topical agents include: clotrimazole, amorolfine or butenafine nail paints. All of these compounds are amenable to incorporation into the fine particulate ion-exchange materials. Topical treatments need to be applied daily for prolonged periods (at least 1 year). For example, terbinafine-modified fine particulate activated ion-exchange polymer salt material may be a candidate for treatment. Incorporation of this salt into a hydrophilic lacquer to be spread onto the nail bed is anticipated to be an appropriate treatment. In another embodiment reflective of the flexibility to mix and match various active ion-exchange species, a laboratory bench to be used for tissue culture for example may be fabricated to include a mixture of antibacterial, antifungal, and antiviral agents thus minimizing the likelihood of contamination of cell lines from environmental contamination.

Antiviral compounds such as acyclovir (a synthetic nucleoside for treating herpes zoster and genital herpes), zidovudine or azidothymidine (a nucleoside analog for treating HIV/AIDS), abacavir (a nucleotide reverse transcriptase inhibitor), and lamivudine (a nucleoside nucleotide reverse transcriptase inhibitor) are readily bound to the fine particulate activated ion-exchange polymer to yield the ion-exchange salt. One potential application for the antiviral-modified fine particulate activated ion-exchange polymer salts is to include the particulate into a hydrophilic matrix for placement into the vagina or anus for the delivery of the drug over time. Both of these locations are ideal for drug deliver due to the high vascularity thus allowing the drug to be effectively administered.

Other candidate active agents for the treatment of parasitic diseases can be incorporated onto the ion-exchange backbones. For example chloroquinine, mefloquine, or doxycycline for the treatment of malaria can be readily bound to IRP69, IRP64, as well as phosphates such as cellulose phosphate. Compounds for the treatment of amoebozoa infections that cause dysentery including azoles (metronidazole and tinidazole), diiodohydroxyquinoline, and paromomycin for example can be employed with IRP69, IRP64, or polyphosphates. Helminth (nematode) infection particularly of the intestinal tract in humans and livestock can be treated using IRP69-, IRP64- or polyphosphate-ion-exchange materials modified to include piperazine, benzamidazoles, levamisole, pyrantel, or morantel. These compositions may be incorporated into materials that may be used as a

In an exemplary embodiment, a water filtration device fabricated from a non-woven fabric (e.g. polyester) filtration units formulated to include one or more of the antimicrobial additives of the present invention may be used in the sanitation of water.

Yet additional advantages afforded by the instant invention include the ability to yield antiparasitic-modified fine particulate activated ion-exchange polymer salt materials in order to provide novel utility and efficacy against infectious parasitic diseases that include treatment of sleeping sickness caused by Trypanosoma brucei) using Melarsoprol-modified material, sleeping sickness using Eflornithine modified material, vaginitis caused by Trichomonas using Metronidazole-modified material, intestinal infections caused by Giardia using Tinidazole-modified material, the treatment of visceral and cutaneous leishmaniasis using Miltefosine-modified material.

The novel biologically activated polymer salts and polymer composites of the invention remain fully biologically active during preparation and for an extended period of shelf life thereafter, even though preparation of the polymer salts in fine particulate form involves non-solvent exposure and temperatures elevated to 85° C. or higher, and despite that curing of the polymer composites often involves elevated temperatures of up to 150 degrees, or 200° C. or higher. In addition, the biologically activated polymer salts and polymer composites remain active with the biologically active ionic agent incorporated therein being stable to degradation, oxidation, chemical decomposition, and photodegradation for an extended shelf period after production as described. Additionally, the novel biologically activated polymer composites of the invention retain not only their biological activity potential, but also their structural integrity for extended shelf and use periods. This activity retention and structural stability is marked by no greater than about 2 to about 5% of chemical loss, degradation, decomposition, destructive hydrolysis or oxidation for the biologically active ionic agents incorporated in the polymer salts and composites, and no greater than about 2 to about 5%, loss of tensile strength, environmental stress cracking, hardness change, or loss of elasticity of the composites during production, including during extended curing of composites at 200° C. with the exception of fine particulate ion-exchange powder salt materials that may interfere with cure for example as a consequence of interference with a catalyst for example. In other embodiments, the stable retention of biological activity structural integrity of these novel polymer composites fabricated as compatible blends, i.e. the fine particulate ion-exchange powder salt material does not interfere with curing of polymer systems or used as matrix materials, is marked by no greater than about 1 to about 5 wt % loss under reasonable operating conditions and when tested alone, the resin systems exhibit remarkable stability well beyond the stability measured using the simple ion salt counterparts of the biologically active component. In general, the fine particulate ion-exchange powder salt materials possess overall greater chemical stability, reduced thermal degradation and decomposition, and greater stability to destructive hydrolysis or oxidation for the biologically active ionic agents incorporated in the polymer salts and composites, and no greater than about 5 to about 15%, loss of tensile strength, change in hardness and/or modulus, or loss of elasticity for the composites over 1-3 months, 6 months, and up to a year or more in normal storage conditions (e.g., at standard laboratory room temperature and humidity, without use or mechanical wear). In more detailed embodiments, activity retention and structural stability is marked by no greater than about 1 to about 20% of chemical loss, degradation, decomposition, destructive hydrolysis or oxidation for the biologically active ionic agents incorporated in the polymer salts and composites, and no greater than about 1 to about 20%, loss of tensile strength, environmental stress cracking, hardness change, or loss of elasticity for the composites following extended exposure (up to 1-3 hours or longer) of the cured or hardened composites to extreme temperatures exceeding 200 degrees, 300 degrees and even 400° C. (allowing for a much broader array of clinical and industrial uses and post-production treatments of these novel composites and biomaterials).

After periods of use, the surfaces of biologically activated composites and related biomaterials of the invention may start to lose their peak biological activity potential. For example, the biologically active ionic agents incorporated in the composites may become partially exhausted due to mechanical abrasion and other mechanisms of loss, ionic dissociation (particularly when used in contact with physiological or other ionic fluids), chemical reaction, chemical change by oxidation or hydrolysis, photodegradation, or other types of removing, discharging, destructive, transforming or deactivating factors.

Among the most surprising and medically advantageous discoveries of the invention herein are materials having a surface biological activity that is “self-recharging”, “self-regenerating”, or “renewable” following an initial period of use (wherein an initial biological activity potential is partially or completely exhausted or discharged). In exemplary embodiments, a fine particulate biologically activated ion-exchange resin material is integrated throughout a solid polymer structure to provide for renewable surface activation following discharge (e.g., due to surface wear or erosion, chemical or ultraviolet degradation of biologically active agents, release or dissociation of activated ion-exchange resin material and/or biologically active ionic agents from the polymer surface, etc.)

In alternative embodiments, the biologically activated ion-exchange resin material is integrated within an outer or inner surface portion only of a solid polymer structure, and may be absent from all or part of deeper internal, core or interstitial portions of the polymer structure. In other alternative embodiments, the biologically activated ion-exchange resin material is integrated within a coating or multi-layer laminate formed of the solid polymer, which can be applied or co-formed to cover a different polymer or non-polymer structure that does not incorporate the biologically activated ion-exchange resin material.

Within these and related embodiments, as the biologically activated ion-exchange resin material, and or the integrated ionic biologically active agent, is discharged, degraded, dissociated or exhausted at the surface of the activated polymer composite (e.g., by mechanical wear or debridement, light or chemical degradation, chemical reaction on contact with external chemical species, oxidation, hydrolysis, decomposition, and/or ionic dissociation of the active ionic agent through exposure to physiological or other ionic fluids, chemical reaction), most or substantially all of an original surface biological activity of the polymer structure is maintained, either passively, for example by “erosive recharging” (wearing that debrides old surfaces and brings out a newly-exposed, fully charged surface), or actively through manual recharging (e.g., manual debridement to expose a new surface with full activity potential, such as by abrasive polishing), or chemical recharging or reconditioning.

In one “self-regenerating” embodiment of the invention, recharging of surface biological activity following partial or complete “discharge” of the ionic biologically active agent initially present (e.g., after the polymer composite is newly formed and hardened) at the polymer surface is achieved by passive erosive recharging. Here, normal contact abrasion (e.g., rubbing of surgical or catheter tubing against another object) wears away an immediate, outermost surface “layer.” This abrades away or debrides the outermost layer, which will frequently comprise some discharged material (i.e., where the outermost layer of polymer exhibits less than the initial loading or activity capacity of the biologically active ionic agent). This exposes a “new”, “regenerated” or “restored” outer layer fully invested with the ionic biologically active agent in a non-degraded or discharged state. In exemplary, “self-disinfecting” embodiments, products incorporating antimicrobially-activated polymer composites of the invention may have an erodible surface and function such that abrasion of the erodible surface exposes new (originally subsurface) antimicrobial particles (activated fine particles of ion-exchange resin material incorporating an ionic antimicrobial agent).

In additional embodiments of the invention, activated polymer composite are provided having “rechargeable” surface structure, chemistry and biological activity after partial or complete “discharge” (including loss of structural or chemical surface active components, chemical degradation of surface active components from an original exposed surface, etc., as described above). In exemplary embodiments, the surface of a biologically activated polymer composite of the invention is rehabilitated or recharged after becoming partly or completely discharged by chemical degradation, decomposition or dissociation of some or all of an initial “surface load” (e.g., surface concentration or titer of exposed metal ions, or ionic molecules, per square inch of exposed surface) or “surface activity potential” (e.g., initial biological activity, such as potential to inhibit microbial contamination, growth or effective re-transmission from the active composite surface).

Restoration the surface of a biologically stable composite material may occur following a natural wearing away by abrasion or other mechanical wearing away of the surface. This may be particularly useful for antimicrobial active materials as most transfer of pathogens in hospital settings involves contact between surfaces. In this instance, the more extensive the contact, the more regenerative activity is provided. In other embodiments, restoration is provided by deliberate manual abrasion or polishing of a subject surface to remove an exhausted outer portion of the material wherein the active agent is set not only within the surface, but within the layers of the polymer surface or throughout the polymer. Abrading and polishing can be done by any number of materials such as abrasive sheets, abrasive pastes, and abrasive gels. Such abrasive and polishing materials may contain different grades of abrasive material with the finest necessary grade leaving the outer surface smooth so that there are no contaminable pores or voids.

In other embodiments, the surface of the biologically active polymer composite may be recharged chemically. For example, biologically active polymer composites comprising oligodynamic metals may become ionically exchanged in physiological fluid causing a loss of the biologically active agent. The surface of the biologically active polymer may be recharged by exposing the surface to an ion-exchange liquid comprising a salt of the biologically active agent such as, but not limited to, silver acetate, copper chloride or copper salt. Exposure of the surface of the biologically stable composite material to an ion-exchange liquid restores about 10 to about 50% of the activity of a new surface of the biologically stable composite material, about 25 to about 75% of the activity of a new surface of the biologically stable composite material, about 15 to about 25% of the activity of a new surface of the biologically stable composite material. Such recharging may take at any time, but is frequently done when the biologically stable composite material has lost about 10%, about 20%, about 25% or more of its peak biological activity.

The surface of the biologically stable composite material may additionally be activated from an original, post-fabrication unactivated state by surface chemical activation (alternatively, surface charging or chemical potentiation). In one exemplary “surface activatable” composite, a “Fenton reaction” is employed externally upon a finished composite surface to activate the surface (and embedded ion-exchange polymer salt components) to generate de novo superoxide from the activated surface. Within these and related embodiments, the fine particulate activated polymer salt comprises and activatable ionic agent, such as ionically associated iron (II). When the polymer composite surface is sprayed, dipped or wiped with a solution of hydrogen peroxide, an activation chemical reaction occurs to generate superoxide at the surface of the composite, yielding a potent surface antimicrobial activation effect. Further, it is known that Fenton's reagent and hydrogen peroxide can be used to oxidize contaminants or waste waters. As such, high surface area substrates coated with oxidation-stable polymer matrixes (such as with a fluoropolymer (Teflon) or rubber such as isobutylene or styrene-isobutylene-styrene and incorporating the activatable IRP69-Fe or IRP69-Cu resin could be placed into holding tanks along with hydrogen peroxide to provide a means of generating superoxide in a controlled fashion while allowing the excess Fenton reagent to be easily removed from the waste water stream.

In one exemplary embodiment, a port of a central venous catheter (CVC) comprising a polycarbonate (female) luer connector fitted with a silicone rubber septum and both components formulated to include fine particulate ion-exchange powder salt in Fe(II) form and at the time of pairing the female luer connector of the CVC with the male luer counterpart for the delivery of medicament or nutrition, the female luer connector is swabbed with a sponge containing hydrogen peroxide solution. The sponge may be fitted onto a male luer connector in order to allow the cap to be turned to rub/swab the hydrogen peroxide moistened sponge across the surfaces of the female luer connector thus enhancing fluid contact and the uniform generation of superoxide as a means of adequately disinfecting the inner surfaces of the connector. This embodiment is described a means of preventing catheter-related blood stream infections (CRBSIs).

In various embodiments, biologically activated polymer composites of the invention can be restored, reactivated, rehabilitated or regenerated after partial or complete discharge to regain 10 to 15% of an initially-loaded, post-fabrication activity potential, 15 to 25% of initial activity potential, 25 to 50% of initial activity potential, 50 to 90% of its initial activity potential, or total initial activity potential or full “recharge” (e.g., where the same level of initial post-fabrication “loading” of functional ion-associating groups on the surface-exposed fine ion-exchange polymer salt particles are effectively “reloaded” with biologically active counter-ion, or otherwise restored (by ion-exchange or chemical reactive restoration, as described). Other means for evaluating restoration of “activity potential” include direct biological activity comparisons (e.g., Kirby-Bauer assays, adhesion assays, biofilm formation assays, colonization assays and the like), for example to test activity potential between initially loaded composites immediately after fabrication, compared with partially discharged or exhausted composites after prolonged storage, use, or exposure to environmental degradation factors (e.g., deionizing, corrosive, oxidative, hydrolytic, chemical reactive, photodegradative, or thermal degradation factors), compared with passively or self-regenerated, mechanically regenerated, or chemically regenerated, restored or rehabilitated composites.

Polishing of surfaces yields freshly active solutions and may be carried out at predetermined intervals. In critical environments, such as in the clinic this may be carried out on a weekly basis for example. In environments where polishing may not be possible, recharging of the surface using a simple “active” salt such as copper (II) chloride or silver nitrate can be accomplished. In order to carry out such tasks perhaps the most logical way to approximate how much active “recharging” agent is needed is to use surface area and in conjunction with the binding capacity of the slat system to undergo recharging. For example, a 24 inch×24 inch×2 inch composite material (22 lbs) that is formulated to include a 5 wt % additive of silver (sulfonated polystyrene-co-divinyl benzene) contains (0.05*22 lb)*454 grams/lb=500 grams. If we assume that a micron thick slice of this composite is what requires recharging we may assume that 4% of the biologically active additive was lost and requires replacement (19.7 grams of additive). If the additive is for example a silver derivative of sulfonated polystyrene-co-divinyl benzene, we can assume that we need approximately 30% of the weight of the additive (95 mmoles) to be recharged with silver. In the case of silver nitrate, this amounts to 16.2 grams of material dispersed into a carrier (water) and allowing the surface to be recharged over some period of time. A separate method would require a first treatment of the surface with HCl and a second treatment with dilute silver acetate. The activity remaining in any particular surface may be determined by surface analysis (evaluation for the particular active ion). This may be done perhaps colorimetrically, using test swabs, strips, of by polishing the surface and evaluating the residue from the polishing process.

In some embodiments, the self-regenerating or rechargeable composites described herein may additionally contain secondary stabilizing materials, for example antioxidants, UV stabilizers, fillers, colorants, fillers and the like.

Various assays and model systems can be readily employed to determine the effectiveness of the fractured copolymeric ion-exchange material with therapeutically useful counter-ions incorporated into polymer matrices. For example, antimicrobial effectiveness may be shown by using a Kirby-Bauer Assay. The Kirby-Bauer Assay (Disk diffusion/Zone of inhibition) is a test method that uses antimicrobial-impregnated wafers to test whether particular bacteria are susceptible to specific antimicrobial agents. In this method, bacteria are grown on agar plates in the presence of samples containing relevant antibiotic agents. If the bacteria are susceptible to a particular antibiotic, an area of clearing surrounds the sample where bacteria are not capable of growing (referred to as a zone of inhibition).

Kirby-Bauer assays can be used to evaluate the effectiveness of the materials (ion-exchange material loaded with oligodynamic metal ions and ammonium ions, and blended silicone LSR materials) and the materials can be shown to possess broad antimicrobial capability against Gram-negative and Gram-positive organisms, and fungi including but not limited to: Staphylococcus, Pseudomonas. Escherichia coli, Klebsiella pneumoniae, Legionella, Mycobacteria, Streptococcus, Acinetobacter, Haemophilus, and Enterococcus. As well as Aspergillis. These agents can be tailored to address multidrug resistant organisms and a variety of airborne pathogens including Mycobacterium tuberculosis and Legionella pneumophila.

Kirby-Bauer assays for a variety of biocides and composites were carried out using the methodology previously described. The results for the biocide powders alone are incorporated into Table 3, the results for RTV curing silicone composites (MED 4955, (Nusil Technology, Carpinteria Calif. 93013) are demonstrated in Table 4, the results for UV-curing silicone composites (Silopren 2060 UV silicone gel (Momentive, Waterford, N.Y. 12188) are included in Table 5, and the results from polyurethane hydrogel composites (TG500, TG500/TG2000 blends) containing SCE and WCE biocides are tabulated in Table 6.

Table 3 below details GARDION™ (IRP64 and IRP69 resin forms) biocide powder activity vs. multiple organisms in Kirby Bauer agar diffusion assays. (x=bacterial surface kill, (o)=no antibacterial activity observed, (-) indicates not tested). Ag=silver, BA=benzalkonium, CHX=chlorhexidine, Oct octenidine, Dox=doxycycline, CP=cetylpyridinium.

TABLE 3
Compiled Kirby Bauer disk diffusion assay results
for GARDION ™ biocide powders

| GARDION ™ | | S. | P. | S. | E. |
|---|---|---|---|---|---|
| Biocide | matrix | Aureus | aeruginosa | Epidermidis | faecalis |
| IRP64-Ag | powder | x | x | — | — |
| IRP64-BA | powder | x | ∘ | — | — |
| IRP64-Cu | powder | ∘ | ∘ | — | — |
| IRP69-Ag | powder | x | x | — | — |
| IRP69-BA | powder | x | x | — | — |
| IRP69-Cu | powder | x | x | — | — |
| Mac 3-Ag | powder | x | x | x | x |
| Mac 3-Cu | powder | x | x | x | x |
| Cell. Phos. Cu | powder | x | x | x | ∘ |

Table 4 Shows antibacterial activity of GARDION™ biocides loaded into MED 4955 silicone gel (Nusil Technology, Carpinteria Calif. 93013) vs. multiple organisms in Kirby Bauer agar diffusion assays. (x=bacterial surface kill, o=no antibacterial activity observed, -=was not tested). Ag=silver, BA=benzalkonium, CHX=chlorhexidine, Oct=octenidine, Dox=doxycyclinc, CP=cetylpyridinium.

TABLE 4
Compiled Kirby Bauer disk diffusion assay results for silicone
composites containing GARDION ™ Biocides

| GARDION ™ | | | | | |
|---|---|---|---|---|---|
| Biocide | | S. | P. | S. | E. |
| (load %) | matrix | Aureus | aeruginosa | Epidermidis | faecalis |
| IRP64-Ag | 4955 | x | x | x | x |
| (5%) | | | | | |
| IRP64-BA | 4955 | x | x | x | x |
| (5%) | | | | | |
| IRP64-CP | 4955 | x | ∘ | x | x |
| (5%) | | | | | |
| IRP64-CHX | 4955 | x | ∘ | x | x |
| (5%) | | | | | |
| IRP64-Oct | 4955 | x | ∘ | x | x |
| (5%) | | | | | |
| IRP64-Dox | 4955 | x | x | x | x |
| (5%) | | | | | |
| IRP69-Ag | 4955 | x | x | x | x |
| (5%) | | | | | |
| IRP69-BA | 4955 | x | ∘ | x | x |
| (5%) | | | | | |
| IRP69-CHX | 4955 | x | x | x | x |
| (5%) | | | | | |
| IRP69-CP | 4955 | — | — | x | — |
| (5%) | | | | | |
| IRP69-Oct | 4955 | — | — | x | — |
| (5%) | | | | | |
| IRP69-Dox | 4955 | — | — | x | — |
| (5%) | | | | | |
| IRP69-Ag/ | 4955 | x | ∘ | x | x |
| BA (5%) | | | | | |
| IRP69-Ag/ | 4955 | x | x | x | x |
| CHX (5%) | | | | | |
| IRP69-Ag/ | 4955 | x | x | x | x |
| Dox (5%) | | | | | |
| IRP69-Ag/ | 4955 | x | x | x | x |
| CHX (5%) | | | | | |
| IRP69-BA/ | 4955 | x | x | x | x |
| CHX (5%) | | | | | |
| IRP69-BA/ | 4955 | x | x | x | x |
| CHX (5%) | | | | | |
| IRP69-BA/ | 4955 | x | x | x | x |
| Oct (5%) | | | | | |
| IRP69-CHX/ | 4955 | x | x | x | x |
| Oct (5%) | | | | | |
| Amberlite Ag | 4955 | x | — | — | x |
| (5%) | | | | | |
| Amberlite BA | 4955 | x | — | — | x |
| (5%) | | | | | |
| Amberlite Cu | 4955 | x | — | — | x |
| (5%) | | | | | |
| Amberlite Oct | 4955 | x | — | — | x |
| (5%) | | | | | |
| Amberlite (1:1) | 4955 | x | — | — | — |
| Ag/BA (5%) | | | | | |
| Amberlite | 4955 | x | — | — | — |
| CHX (5%) | | | | | |

Table 5. Shows antibacterial activity of GARDION™ biocides loaded into Silopren 2060 UV silicone gel (Momentive, Waterford, N.Y. 12188) vs. multiple organisms in Kirby Bauer agar diffusion assays. (x=bacterial surface kill, o=no antibacterial activity observed, -=was not tested). Ag=silver, BA=benzalkonium, CHX=chlorhexidine, Oct=octenidine, Dox=doxycycline, CP=cetylpyridinium.

TABLE 5
Compiled Kirby Bauer disk diffusion assay results for UV-cured
silicone composites containing GARDION ™ Biocides

| GARDION ™ | | | | | |
|---|---|---|---|---|---|
| Biocide | | S. | P. | S. | E. |
| (load %) | matrix | Aureus | aeruginosa | Epidermidis | faecalis |
| IRP64Ag | 2060 | x | x | — | — |
| (10%) | | | | | |
| IRP69Ag | 2060 | x | x | — | — |
| (10%) | | | | | |
| Amberlite | 2060 | x | — | — | x |
| Ag (5%) | | | | | |
| Amberlite | 2060 | x | — | — | x |
| BA (5%) | | | | | |
| Amberlite | 2060 | x | — | — | x |
| Cu (5%) | | | | | |
| Amberlite | 2060 | x | — | — | x |
| Oct (5%) | | | | | |
| Amberlite (1:1) | 2060 | x | — | — | — |
| Ag/BA (5%) | | | | | |

Table 6 shows antibacterial activity of GARDION™ biocides loaded into Lubrizol TG500, TG2000, and 1:1 TG500/TG2000 blends (Lubrizol, Cleveland Ohio) vs. multiple organisms in Kirby Bauer agar diffusion assays. (x=bacterial surface kill, o=no antibacterial activity observed, -=was not tested). Ag=silver, BA=benzalkonium, CIIX=chlorhexidine, Oct=octenidine, Dox=doxycycline, CP=cetylpyridinium.

TABLE 6
Compiled Kirby Bauer disk diffusion assay results for
polyurethane hydrogels (TG500 and TG2000 (Lubrizol
Inc.. Cleveland OH) containing GARDION ™ Biocides

| GARDION ™ | | | | | |
|---|---|---|---|---|---|
| Biocide | | S. | P. | S. | E. |
| (load %) | matrix | Aureus | aeruginosa | Epidermidis | faecalis |
| IRP64Ag | TG500, | x | x | — | — |
| (10%) | 1:1 | | | | |
| | TG500/ | | | | |
| | TG2000 | | | | |
| IRP69Ag | TG500, | x | x | — | — |
| (10%) | 1:1 | | | | |
| | TG500/ | | | | |
| | TG2000 | | | | |
| Amberlite | TG500, | x | — | — | x |
| Ag (5%) | 1:1 | | | | |
| | TG500/ | | | | |
| | TG2000 | | | | |
| Amberlite | TG500, | x | — | — | x |
| BA (5%) | 1:1 | | | | |
| | TG500/ | | | | |
| | TG2000 | | | | |
| Amberlite | TG500, | x | — | — | x |
| Cu (5%) | 1:1 | | | | |
| | TG500/ | | | | |
| | TG2000 | | | | |
| Amberlite | TG500, | x | — | — | x |
| Oct (5%) | 1:1 | | | | |
| | TG500/ | | | | |
| | TG2000 | | | | |
| Amberlite (1:1) | TG500, | x | — | — | — |
| Ag/BA (5%) | 1:1 | | | | |
| | TG500/ | | | | |
| | TG2000 | | | | |

Efficacy may additionally be demonstrated through the use of ISO 22196. ISO 22196, Measurement of antibacterial activity on plastics and other non-porous surfaces, has been utilized for the evaluation of antimicrobial ion-modified resins incorporated into a variety of different materials. These antimicrobial ion-exchange modified materials have demonstrated between 3-Log to7-log overall reductions in bacterial (organism) counts for species such as Escherichia coli and Staphyloccocus aureus at as little as 1.0 wt % loading levels (% by weight of activated fine particulate polymer salt per final composite weight, determined prior to mixing of polymer salt with thermoplastic or thermoset polymer).

Efficacy of the biomaterials provided herein may be demonstrated, for example, through the use of ASTM E2180-07 (ASTM International, West Conshohocken, Pa., 2007). ASTM E2180-07 is a method whereby treated test samples are inoculated with the test organism mixed within a semi-solid agar “slurry” to facilitate surface interaction. The test organism is thus exposed for attachment/colonization on the surface of the test material typically for 24 hours. Control samples of the same material that is not “activated” according to the invention (e.g., a silicone polymer that does not contain activated fine particulate polymer salt material) is similarly inoculated and tested. The test and control samples are then treated with a neutralizing solution comprising tryptic soy broth (base), lecithin (1.0 gram/liter) and Tween 80 (7.0 grams/liter). With this solution, cationic antimicrobial agents are neutralized in order to prevent them from continuing to eliminate bacteria during the test procedure, the surfaces are subsequently washed and samples are quantitatively assayed for antimicrobial activity (e.g., bactericidal and/or bacteriostatic activity). The resulting plates are incubated, and the number of survivors can be enumerated by direct surviving cell counts and/or by determining both survival and viability for reproduction through subsequent detection of colony production (colony forming units or CFUs). This provides for measurement and expression of “decontamination efficacy” of the novel biomaterials of the invention, which may be expressed as a percent reduction of viable microbes capable of surviving and/or reproducing. These values are determined for both test and control materials, and on this basis relative efficacy values for “decontamination activity”, bactericidal and/or bacteriostatic activity, and “transfer risk reduction”, among other measures of efficacy, can be determined. Comparable assays are routinely implemented to determine antifungal (fungicidal and fungistatic) activity, antiviral activity, and antiprotozoan (e.g., amebicidal) activity.

Common test organisms utilized in these method for determining antibacterial activity include Escherichia coli and Klebsiella pneumoniae. Exemplary antimicrobial polymer composites of the invention have been tested and shown to effect 3.69 and 3.72 log reductions against these bacteria, respectively. In other exemplary embodiments, antimicrobial polymer composites having as low as 1.0 wt % loading of the composite with fine particulate activated ion-exchange polymer salt have been tested and shown to effect 6.2 and 5.98 log reductions in these respective organisms at as little as 1.0 wt % loading. The data from these and other assays demonstrate the ability of activated ion-exchange polymer salts and polymer composites incorporating these novel materials as potent drug delivery and surface active biomaterials for use in clinical, industrial and other applications. The tables herein depict antibacterial activity results for silicones incorporating fine particulate activated polymer salt particles (IRP69) comprising biologically active counter-ions of Ag evaluated over a four week period during which time the samples were extracted in 0.9% normal saline at 37° C. during the time course of the study.

In certain aspects of the invention, biological activity potential of activated polymer composites can be varied by selecting different effective loading amounts particle distributions within composites for the activated, fine particulate ion-exchange polymer salt. Biologically effective amounts (or ratios) of the polymer (e.g., per wt % of its incorporation within polymer composite mixtures) can be selected across a broadly validated range. For example, polymer composites comprising as little as 1 wt %, to as much as 75 wt % or higher, of the fine particulate ion-exchange polymer provide active composites with acceptable structural, cosmetic, stability, and performance characteristics. In certain embodiments, a selected weight percentage of the fine characteristics. In certain embodiments, a selected weight percentage of the fine particulate ion-exchange polymer salt incorporated within useful polymer composite mixtures are selected as a “biologically effective amount” (by wt %) to mediate a specific biological activity potential (translatable to all biological activities described herein). In exemplary embodiments, an effective amount of a fine particulate ion-exchange polymer salt incorporated within a polymer composite may mediate antimicrobial activity potential characterized by an ability of the polymer composite to inhibit specific microbial survival, growth and/or transmission potential to a second surface or living subject. For example, effective amounts of fine particulate ion-exchange polymer salts in certain polymer composites will increase zones of bacterial inhibition by 10%, 20%, 30%, 50% or greater, up to 75-90%, or 95% or greater, compared to comparable inhibition activity measures determined for an unactivated composite (i.e., a like composite not incorporating activated fine particulate ion-exchange polymer salt—either having no particulate polymer material, or having like particulate ion-exchange material in like amounts not activated by incorporation of biologically active ionic agent). In other embodiments, effective amounts of fine particulate activated ion-exchange polymer salt will mediate inhibition of bacterial biofilms, bacterial reproduction, and/or bacterial transmission from a contaminated composite surface to a secondary surface or live subject by 10%, 20%, 30%, 50% or greater, up to 75-90%, or 95% or greater. Comparable levels of selectable activation potential for all activities imparted to the novel polymer composites of the invention (e.g., antifungal activity, antiviral activity, anti-inflammatory activity, etc.) are similarly achieved using selectable effective amounts of fine particulate polymer salt materials within different activated composites, according to the description herein.

Activated polymer composites of the invention can be formed as flexible or rigid biomaterials in virtually any shape, size, thickness or structural relationship with other materials (e.g., Teflon, nylon PTFE, stainless steel, titanium, etc.) to make biomedical articles, tools and devices. The polymer composites may incorporated into biomaterials, textiles and articles of manufacture, for example, by casting, molding or assembling the composites directly into an article of manufacture, coating or laminating the composites over articles of manufacture, or mixing the composites with textiles or other precursors of articles of manufacture, among other fabrication modes and formulae.

Accordingly, the biologically activated polymer composites of the invention are useful to form integral, internal or external components, infused or permeated media, lattices and textiles, laminates and coatings, etc., to provide novel structural and biological advantages to a diverse array of medical, veterinary, dental, orthopedic and laboratory materials, devices equipment and furnishings. The novel biomaterials and composites of the invention may make up the products in their entirety by molding, curing, or other fabrication means, or they may be coated, laminated, over-molded, or coextruded onto other materials, components or products. In exemplary embodiments, components and products are made from activated polymer composites of the invention by transfer molding, extraction molding, extrusion molding, blow molding, or other molding techniques. In other exemplary embodiments, biomaterials and articles of manufacture are produced by forming the solid composites as sheets, which may in turn be applied to or adhered to a different material, substrate, component or product. Coatings comprising biologically activated polymer composites of the invention may have the same thickness over an entire material or product profile or surface, or be coated onto a material or product in varying thicknesses at different sites or functional parts, depending on use.

The invention thus provides a valuable assemblage of biologically activated polymer composites for construction of clinical, therapeutic and diagnostic materials and devices. Operative embodiments employ the biologically activated polymer composites of the invention incorporated within such diverse materials and devices as antimicrobial disposable blotters, sponges, and surgical wear (e.g., gloves and shoe covers), permanent or temporary coverings for traditional fomite surfaces such as surgical trays, operation room (OR) equipment, drug and fluid delivery devices, catheters and tubing, cardiovascular and orthopedic implants, stents, grafts, and anchoring or suturing materials and devices (e.g., pins, posts, staples, and sutures) and a diverse array of comparable laboratory equipment (e.g., materials, components, tools, containers, disposable and non-disposable coverings and textiles for use in forensic, diagnostic, microbiological and tissue culture laboratories).

Additional biomaterials, components, coatings, devices, furnishings and equipment in which the novel activated polymers of the invention are beneficially incorporated include, for example, food-processing equipment, packaging and products; consumer clothing and apparel; first responder protective wear and gear; athletic (e.g., sports therapy and gymnasium) materials, equipment and clothing; lavatory materials, furnishings and equipment, transportation equipment (e.g., high-contact/heavy use surfaces on buses, subways, trains, planes, cruise ships), and HVAC and other air and fluid circulation and management systems and components (e.g., coatings on air ducts, connectors, ports, collectors, fan blades and housings, impellers and filters).

Exemplary medical, laboratory and industrial materials and devices of the invention include activated polymer composites integrated within paints, floor coverings, wall materials, joining and adhesive compounds for walls and furnishings, countertops, laminate materials, filters, and appliances. Exemplary medical and laboratory devices and equipment that can be partially or completely constructed of the novel biomaterials provided here include drug and fluids delivery and catheter tubing, molded components, coatings, surgical tools and equipment, biohazard disposal surfaces and containers, hospital bedding, gurneys, stretchers, textiles including surgical scrubs, gowns, surgical drapes, bedding, wound dressings, etc. Other, similar assemblages of materials, devices and applications are contemplated for food harvesting, handling, processing and serviced industrial tools, textiles and equipment, and for heating, ventilation, and air conditioning (HVAC) system components including filters, heat exchangers, coils, duct work, fans, humidity control components, heat pumps, vents, manifolds and the like. Yet additional materials, devices and applications will incorporate the activated polymer composites of the invention within bulk storage containers, public transportation surfaces, office equipment, food conveyers, clean rooms, consumer products (children's toys, high chairs, bathroom cleaning appliances, sexual prosthetics (e.g., vibrators, dildos, erectile dysfunction aids and the like), hygiene implements such as toothbrushes, dental floss and skin and eye care materials and devices).

Exemplary medical and hygiene products that will beneficially incorporate biologically activated polymer composites of the invention include, for example, catheters, tracheostomy tubes, wound drainage devices, stent, implants, introducers, stylets, sutures, shunts, gloves (latex, neoprene, viton), condoms (polyurethane, latex, silicone), contact lenses, gastrostomy tubes, cardiovascular stents, prostheses, pacemakers, grafts, valves and implants, surgical guidewires, urine collection devices, medical tubing, intravenous catheters, urinary catheters. Foley catheters, pacemaker leads, urological catheters, wound dressings, medical sheeting, endotracheal tubes, septae used for piercing with needles for sterile retrieval of drugs from supply vials, or for delivery of drugs, nutrients, saline or other materials via i.v., connectors, clamps, shunts, catheter ports, hubs, catheter port cleaning cap devices (for ensuring that septum and port are sterile for the providing drug therapy, nutrition, or removing body fluid), surgical repair constructs and meshes, and many other materials and devices.

Exemplary sexual prostheses include dildos, vibrators, sleeves and other stimulatory devices, male and female artificial flesh products, erectile dysfunction aids including suction devices and implants, compression rings, as well as any other adult sexual device or prosthetic designed for intimate mucosal contact or penetration, as may be fabricated, e.g., from silicone, polyurethane or other soft flexible hypoallergenic materials.

Exemplary contraceptive devices that will benefit from the inclusion of biologically activated polymer composites of the invention include intrauterine devices (IUDs) comprising a copper derivative form (SO₃⁻, CO₂⁻, OPO₃⁻). Paragard® is a known IUD that releases small amounts of Cu⁺⁺ from a copper filament and is known to be safe.

Another exemplary contraceptive device embodiment includes sponges that releases benzalkonium and cholalic acid (cholate) for placement into the vaginal tract. The high surface area device is conducive to having activated fine particulate polymer salt additives incorporated without having any effect on the mechanical performance of the device.

In yet another exemplary embodiment of a vaginal sponge, the acid form of sulfonated polystyrene divinylbenzene or the acid form of the polymethacrylic acid-co-divinylbenzene activated fine particulate polymer salts may be added to a flexible polymer matrix as a means of having an effect on the local pH within the vaginal tract. This will allow for the high surface area sponge to generate hydronium (Note: benzene sulfonic acid has a dissociation constant of 10³) which will affect the local pH (decreasing) at the entrance to the cervix. Because sperm require high pH in order to function properly, such a device will decrease sperm motility. Silicone and or polyurethanes are appropriate materials for such an application. The same strategy can be applied to a diaphragm noting that silicones and polyurethanes are the appropriate materials for diaphrams and sponges.

In yet another exemplary embodiment activated fine particulate polymer salts modified to include spermicidal agents such as benzalkonium and/or cholalic acid and the additives blended into a flexible polymer matrix such as latex and condoms can be fabricated by a dipping process. In some embodiments only the outer layer or layers may include the spermicidal agent depending upon the number of dipping processes required to produce the condom.

All of the exemplary contraceptive devices described in this invention can be further modified to include antiviral agents to minimize the likelihood of transmission of HIV during intercourse.

Among significant industrial and public utilities uses, the biologically activated polymer composites of the invention are particularly well adapted for useful integration in air and water-handling systems, including heating, vacuum, and air conditioning (HVAC) components, conduits, fittings, filters, recirculators, pumps and the like. The heating, vacuum, or air conditioning components can include one or more of duct work, heat exchange coils, heat exchangers, fan components, vents, energy-recovery ventilators, blower components, ballasts, levers, air filters, water filters, heat pumps, fluid handling systems and/or the like.

In other embodiments, the biologically activated polymer composites of the invention are uniquely adapted for improving safety and performance of building, flooring and surface construction materials, including hospital, laboratory and home building, construction and sealing and adhesive materials. Among such materials that will beneficially incorporate surface paints or coatings of these activated polymer composites are flooring materials, countertop materials, and wall construction materials. One exemplary use for these embodiments will be to fight toxic mold encroachment in homes, hospitals and extended care facilities, e.g., by coating indicated building materials, such as gypsum drywall, with polymer composites integrating antifungally active ionic agents.

With regard to construction of biologically activated textiles, the polymer composites of the invention can be used to construct finished fabrics derived from naturally occurring fibers or man-made materials, or from plant-based materials such as paper. The fabric materials can be constructed from one or more of a weave, knit, knot, crochet, or melt spun or unwoven (non-woven fabrics) and the antimicrobial additives of the present invention can be incorporated by inclusion into the fibers of manmade material prior to fabrication of yarn, thread or the like or the antimicrobial additives of the present invention may be added as a coating (sizing) onto the fabric. The textiles as described herein may be utilized to fabricate any variety of textile-based products to include clothing and garments such as shirts, socks and stockings, and pants that may find applications for example in sportswear, and military applications. Garments for use in hospital and healthcare environments may include surgical scrubs, neckties, and lab coats, as well as hospital gowns, pajamas, and undergarments for example. Other textile-based articles can include surgical masks, booties, and protective suiting for application in and around infectious diseases.

In other embodiments, the self-disinfecting compositions may be used to make touch surfaces for use in one of a clinic, hospital, nursing home, long-term care facility, gymnasium, sporting facility, workout facility, kitchen, bathroom, recreation center, academic institution, cafeteria, watercraft, motorized vehicle, and/or disposal container. Touch surfaces as related to gymnasiums, recreation centers, and sporting institutions can include for example grips related to equipment and exercise machines, mats for stretching, martial arts, boxing, and wrestling.

In other exemplary embodiments the activated fine particulate polymer salts may be incorporated into adhesives and sealers for use in building construction materials in order to impart surface or bulk antimicrobial properties to the materials. For example, roofing materials may be susceptible to fungal growth and/or rot. Thus the incorporation of a fungicidal activated fine particulate polymer salts, such as IRP69-Cu can alleviate such a problem. Further embodiments include marine paints to prevent or eliminate the attachment of crustaceans, shipworms and other marine “fouling” organisms (that can decrease efficiency of vessels and degrade marine structures such as ship hulls, docks and bulkheads).

The invention provides a diverse array of biologically activated polymer composite paints and coatings, for use in wide range of applications ranging from clinical and institutional surface coatings and paints, to marine antifouling paints and coatings. In exemplary embodiments, paints and other coating composites are made by admixing with the fine particulate, biologically activated polymer salt with one or more conventional polymers used in manufacturing paints and other surface coatings. These polymers likewise can be provided as thermoset, thermoplastic, photocuring or other curable polymer precursors, though typically the subject paints and coatings will be cured by ordinary drying (e.g., by allowing a solvent present in a liquid composite mixture (aqueous or organic solvent), to evaporate under normal drying conditions after the paint has been sprayed, brushed or otherwise coated onto a surface. Under these conditions, polymer precursors within the polymer composite mixture polymerize and/or cross-link to provide a cured or hardened (i.e., “solid) coating that is bacteriocidal, fungicidal, bacteriostatic, fungistatic, anti-microbial (including anti-protozoan) and/or antifouling (e.g., prevents or deters marine larval settlement and/or growth of marine fouling organisms, such as barnacles and shipworms).

Polymer composites of the invention produced as paints and coatings may be made using a wide range of polymer types, including mixtures of polymers. In exemplary embodiments, polymer precursors may include one or more polysiloxane, polyalkylene, polyamide, epoxy, polycarbonate, polyester, vinyl, acrylic, polyurethane, plastisol (e.g., a suspension of polyvinylchloride or PVC), or polyvinylidinefluoride (PVDF) polymer, or mixtures thereof. These selected polymers for making biologically activated polymer composites of the invention may be present in a pre-mixed commercial paint base, which may include any of a wide range of conventional paint base ingredients-including, for example, multiple polymer types, colloid-promoting agents such as surfactants, preservatives, coloring agents, buffering agents, and the like. Paints and coatings of the invention may be water-based (e.g., latex or acrylic paints and coatings) or solvent-based (e.g., lacquer or epoxy paints and coatings). Any compatible polymer or other additive can thus be employed to produce anti-biologic paints and coatings, which may be provided as an acrylic, latex, polyester, varnish, shellac, glaze, enamel, lacquer, epoxy, plastisol, or PVDF-based paint or coating, while it will be understood all compatible mixtures of polymers and additives from these conventional paint or coating bases may be readily integrated and tested for operability and specific performance effects within the anti-biologic paints and coatings of the invention.

The invention provides a diverse array of more broadly classified “anti-biologic” paints and coatings, including “antifouling” paints and coatings. As used herein, antifouling paints and coatings prevent colonization and/or long-term residence, and/or reduce growth of undesirable organisms. Certain antifouling paints and coatings of the invention will be applied to prevent bacterial or fungal fouling, for example by applying the paint or coating onto a dry, exposed clinical or institutional surface (e.g., a hospital or prison structural surface, such as a wall or fixture, or on furnishings, equipment, ductwork, pipes (or other ventilation or plumbing surfaces, such as fans, screens, filters, valves, etc.), appliances, etc., contained therein.

In certain aspects of the invention, antifouling paints and coatings are provided that provide long lasting anti-biologic effects in marine and/or fresh water applications. Early marine antifouling coatings were tin-based coatings, but such coatings have now been removed from use due to toxicity and environmental concerns. In more recent developments, hydrophobic performance coatings have alternatively been used for marine antifouling applications, consisting of silicone-like polymers, epoxies, or other vulcanizing systems that steadily release antifouling biocides. A common problem with all biocidal antifouling paints and coatings, generally relates to undesirable toxicity and adverse environmental effects. Thus, U.S. Pat. No. 3,214,280 reports a marine antifouling paint composition containing a copolymer of vinyl chloride-vinyl acetate-vinyl alcohol as a film forming paint base, volatile solvent, and 1,2,3-trichloro-4,6dinitrobenzene as an antifouling agent. Another reported antifouling paint composition described in WO 2012150360 A2 teaches copper based biocide incorporated in a binding polymer

Various well known and conventional methods can be routinely employed to determine fresh water or marine antifouling efficacy of the paints and coatings of the invention. These diverse methods test, for example, efficacy for inhibition of marine larval settlement and/or marine organism growth on painted or coated test surfaces immersed in natural or artificial media (e.g., seawater). Useful negative controls will employ like base-paint materials not containing an active biocidal agent. Positive controls for testing the marine antifouling paints and coatings of the invention may include current antifouling paints, for example an industry-leading solvent-based antifouling paint, Micron Extra Red (from International Paint). Test samples of a polymer composite antifouling paint or coating of the invention are applied to plywood, metal or other substrates, and subjected to side-by-side study comparison with comparable positive and negative control coatings. The test and control samples may be immersed, for example, in natural seawater in a subtidal or tidal marine environment, and periodically assessed for settlement and growth of marine fouling organisms (e.g., micro- and macro-algae, marine microbes, soft-bodied animals, and hard-bodied animals). In illustrative embodiments, the marine antifouling paints and coatings of the invention inhibit from at least 10%-20%, often 20-75%, 50%-85%, and up to 95%-100% of the relative fouling observed in positive control samples, and this antifouling efficacy persists for 3-6 months, 6-months to one year, 1-3 years and longer (depending on the construction, loading and thickness of the coating, among other variables that are selectable/adjustable as described herein).

Additional supportive description pertaining to certain aspects and embodiments of the invention may be found, for example, in “Compositions And Methods For Promoting The Healing Of Tissue Of Multicellular Organisms” U.S. patent application Ser. No. 12/162,990, filed Jul. 31, 2008, PCT Patent Application Serial No. PCT/US07/02780, Jan. 31, 2007, to David Vachon, which claims priority benefit of U.S. Provisional Patent Application Ser. No. 60/764,033, filed Jan. 31, 2006; “Compositions And Methods For Promoting The Healing Of Tissue Of Multicellular Organisms” U.S. patent application Ser. No. 12/690,081, filed Jan. 19, 2010, which is a Continuation-In-Part of U.S. patent application Ser. No. 12/162,990, filed Jul. 31, 2008, which is a 371 of PCT/US07/02780, filed Jan. 31, 2007, to David Vachon which claims priority benefit of U.S. Provisional Patent Application Ser. No. 60/764,033, filed Jan. 31, 2006; and “Biologically Efficacious Compositions, Articles of Manufacture and Processes For Producing And/Or Using Same” U.S. patent application Ser. No. 13/532,562, filed Jun. 25, 2012, to David Vachon, which claims priority benefit of U.S. Provisional Patent Application Ser. No. 61/501,086, filed Jun. 24, 2011, U.S. Provisional Patent Application Ser. No. 61/616,332, filed Mar. 27, 2012, each of which is incorporated herein by reference in its entirety for all purposes.

### EXAMPLES

Exemplary compositions, methods, materials and devices of the invention are provided here, which are not to be construed to limit the scope of the invention. The claims of the application are supported by the entirety of the disclosure as well as these examples.

All ion-exchange materials for use within the invention can be purified prior to, or following association with, biologically efficacious counter-ion materials described. In certain exemplary embodiments, ion-exchange materials are received from a commercial supplier and employed as received, or pre-conditioned for example by extraction with isopropyl alcohol prior to air and/or vacuum drying. All matrices such as polymer matrices used in the fabrication of the compositions such as silicone rubber, were prepared according to supplier specifications.

### Example 1

### Production of Strong Cation-Exchange Materials

Many strog cation-exchangers are commercially available, for this example 1RF69-Na was chosen (Dow Chemical Company, Midland, Mich.). The sodium form of the strong cation-exchanger was stirred in a molar excess of 2M HCl three times for 45 minutes using a mechanical stirrer. The solid was then washed with deionized water between each step until the pH was neutral. The wet solid was stored wet in an air tight container under 25° C. away from light. The acid form of the strong cation-exchanger needs to be stored wet in a cool storage container, if the resin is dry or heated it was found to decompose, releasing free acid. This decomposition can be observed by observing color change or a drop of pH in a aqueous solution containing the acid resin. Development of this process has shown the acid form of the strong cation-exchanger is unstable after it has been in contact with alcohols, causing degradation of the acid and the production of ethers while in contact with water.

### Example 2

### Formation of IRP69-H

Many strong cation-exchangers are commercially available, for this example 1RF69-Na was chosen (Dow Chemical Company, Midland, Mich.). The sodium form of the strong cation-exchanger was stirred in a molar excess of 2M HCl three times for 45 minutes using a mechanical stirrer. The solid was then washed with deionized water between each step until the pH was neutral. The wet solid was stored wet in an air tight container under 25 C away from light.

### Example 3

### Determination of Loading Capacity of Strong Cation-Exchanger

A resin storage container was shaken to make a homogenous distribution of moisture. A 1 gram sample of the acid strong cation-exchanger described above was analytically weighed out and placed in a glass column. The resin was quenched through the column using 300 ml of 0.5M Na₂SO₄ followed by 50 ml of DI water. The collected filtrate was titrated with 0.1M NaOH and phenophthalein to the endpoint to determine loading capacity per gram.

### Example 4

### Formation of IRP-69-Ag

Amberlite IRP69 strong cation-exchange material was stirred in a minimal amount of deionized water and an excess (˜10-500 molar excess) silver nitrate was added and the mixture stirred by the addition of a mechanical stirrer for 60 minutes. The solid was filtered washed with copious amounts of deionized water (until the filtrate does not contain any silver nitrate) as evidence testing using Silver Check II HR test strips (Industrial Test Systems, Inc., Rock Hill, S.C. 29730) of the filtrate. The modified IRP69 was dried under vacuum at 130° C. and the material was milled with an IKA homogenizer and the resulting particles were put through a sieve with a 35 μm cutoff. The powder was dried under vacuum and used for incorporation within various polymer composite mixtures. Thermogravimetric analysis of IRP-69-Ag as demonstrates that the little degradation of IR69F-Ag below 400° C.

Another method of synthesizing the activated fine particulate polymer salts involves the use of titrated wet form of Amberlite IRP69F-H (acid form) strong cation-exchange material which has never been dried can be stirred in a minimal amount of deionized water and a molar equivalent or excess amount of the acetate salt (containing the cation of interest, such as silver acetate, zinc acetate, iron acetate, copper acetate, or organic acetates to include chlorhexidine diacetate for example). Following the addition of the acetate salt the mixture can be stirred using a mechanical stirrer for 1-24 hours depending on size of the reaction. The solid can then be filtered, washed with copious amounts of deionized water (until the filtrate does not contain the cation of interest silver acetate (for example) as evidenced from silver test strips (Macherey-Nagel, Bethlehem, Pa.). The pH of the wash was also monitored using pH test strips in order to gauge the presence of byproduct HOAc and the washes can be continued until the pH is neutral. The activated IRP69 resin can then be dried under vacuum at 130° C. and the material was milled using a Retsch PM100CM planetary mill. The powder was dried under vacuum and used for incorporation within various polymer composite mixtures. Yields of the modified resin approach 100% (of cation-exchange capacity) using this method.

### Example 5

### Preparation of IRP64-Potasium

The acid form of a weak cation-exchanger such as Mac-3 or IRP64-H (Dow Chemical Company Midland, Mich.) was stirred in a deionized water solution by a mechanical stirrer. A 1:1 molar equivalence of potassium carbonate (or equivalent sale of interest such as sodium carbonate, barium carbonate, calcium carbonate, lithium carbonate, barium carbonate, iron carbonate, copper carbonate, silver carbonate, zinc carbonate, or magnesium carbonate) was titrated slowly into solution to control the evolution of CO₂ during the reaction. When no more bubbles evolve the reaction is complete. The Resin was continuously washed with copious amounts of deionized water until the filtrate pH is neutral. The resin was then placed in a vacuum at 100 C until dry, yielding a weak cation-exchanger associated with the corresponding metal from the starting carbonate in this case potassium).

### Example 6

### Preparation of IRP64-Ag

In another procedure, Amberlite IRP69-Na+ strong cation or Amberlite IRP64(sodium form) ion-exchange material was stirred in a minimal amount of deionized water with an equal amount of isopropanol. In this example additional salt species (amonium, potassium, magnesium or lithium salt) of the weak or strong cation-exchanger can be used as a substrate. A molar equivalent or small excess of the salt (containing the cation of interest such as silver, benzalkonium, benzethonium, cetylpyridinium, galium, iron, copper, zinc, cysteamine, chlorhexidine, minocycline, tetracycline, tobramycin or gentamicin, in a desired salt form such as sulfate, nitrate, acetate, chloride, hydrocholoride, hydorbromide, or hydrogeniodide), is added and the mixture and heated to 65° C. (optimal temperatures are between 20-70° C.), then continuously stirred with a mechanical stirrer for up to 72 hours. The activated ion-exchange material was subsequently filtered and washed with isopropanol and deionized water until the filtrate did not contain any residual starting material in solution by a test strip or UV-Visible spectroscopy. The modified IRP69 or IRP64 was dried under vacuum at 70-130° C. and the activated resin was milled using a Retsch PM100 planetary ball mill with appropriate grinding media (yielding a particulate activated resin product milled to approximately 100-1000 nm particle sizes as measured by light scattering).

### Example 7

### Use of Acid Form Strong Cation-Exchangers

An acid form of strong cation-exchanger IR69F-Na (Dow Chemical Company, Midland, Mich.) was stirred in a minimal amount of deionized water by a mechanical stirrer in the absence of light. A molar equivalent of minocycline free base (which can be substituted with an equivalent free base containing an amino group or another nitrogen containing molecule able to be protonated, such as tetracycline) was added to the solution which was allowed to stir for 1 hour. The solid was rinsed with copious amounts of deionized water until no acid or minocycline was present in the filtrate. The resin was then titrated slowly using sodium acetate (or a similarly acetate salt such as silver, copper, or zinc) until the solution began to turn a pale yellow from the released tetracycline off the solid material, showing no free acid was available to exchange. The solid was transferred to a vacuum oven at 70° C. for 24 h or until dry producing a light- and heat-stable modified tetracycline resin salt. This material was then milled to I-10 micron particle size as measured by light scattering to produce a fine particulate, biologically activated polymer salt according to the invention.

### Example 8

### Silicone Gel Polymer Composite Containing IRP-69-Ag

MED 6345 Silicone Gel (Nusil Silicone Technology, Carpinteria Calif.) was used as a source of polymer precursors to formulate a polymer composite mixture of the invention, the polymer precursors comprising 12 g part A MED 6345 (catalyst prepolymer)+12 g part B (crosslinking prepolymer). To this cross-linkable polymer precursor blend was added 2.67 g IRP69-Ag (10% w/w), and the resulting biologically activated polymer composite mixture was homogenized using a speedmixer, poured into a mold and air bubbles were allowed to escape (˜20 min). The polymer composite mixture was thereafter cured at 70° C. for 1 hr. According to the novel methods herein, curing of the polymer composite mixture was not inhibited by incorporation of the fine particulate, ion-exchange polymer salt containing biologically active silver. Other known antimicrobial silver compositions would be expected to inhibit curing of silicones and other polymers. In one mechanism, free silver ions available in prior antimicrobial silver formulations would likely impede catalyst function essential for polymerization of silicone and other polymers. In other catalytic polymerization reactions, simple salts of oligodynamic metals, such as silver, and other ionic biologically active agents described herein, are expected to block or impede a variety of different curing mechanisms, including various mechanisms mediated by electron transfer (for example, photocuring of silicones and other polymers, and free-radical mediated cross-linking of other polymers). In the novel polymer composite mixtures of the invention, these curing inhibitory mechanisms are surprisingly prevented or greatly reduced by ion-exchange protection, chelating or shielding of otherwise reactive ionic agents contained in the fine particulate activated ion-exchange polymer salt particles (e.g., as compared to inhibition of curing mediated by their respective simple salt forms).

### Example 9

### Silicone Gel Polymer Composite Containing IRP-69-Benzalkonium

MED 6345 Silicone Gel (Nusil Silicone Technology, Carpinteria Calif.) polymer precursors (3.07 g part A+3.07 g part B) were combined with 0.3231 g with IRP69-Benzalkonium (IRP69-BA) (5% w/w) and mixed by hand to form an activated polymer composite mixture. This mixture was poured onto release liner and air bubbles were allowed to escape (˜20 min), followed by thermally-accelerated curing of the composite mixture at 84° C. for 23 min. Cure was complete and not inhibited by the presence of the quaternary ammonium group present in the benzalkonium compound. This result is particularly surprising, as it has been previously understood that amino compounds, generally, are likely to inhibit curing of silicone-based polymers. Notably, benzalkonium (typically provided benzalkonium chloride), is present in the activated polymer salts of the invention ionically associated with an activated resin, and free chloride counterion has been displaced/removed by ion-exchange. If simple benzalkonium chloride salt were integrated in a silicone polymer, the active agent would melt to liquid form during curing (at its melting point of 35° C.) and disrupt silicone polymerization through one or more of the curing inhibitory mechanisms described above.

### Example 10

### Silicone Gel Polymer Composite Containing IRP69-Cetylpyridinium (CP)

MED 6345 Silicone Gel (3.02 g part A+3.02 g part B) prepolymers were combined with 0.3179 g IRP69-CP (5% w/w), mixed by hand, poured onto release liner and air bubbles allowed to escape (˜20 min). Curing at 84° C. for 23 min. was complete and not inhibited by the ammonium composition of the active compound reactively shielded within the fine particulate polymer salt component of the polymer composite mixture.

### Example 11

### Silicone Gel IRP69-Octenidine Polymer Composites

MED 6345 silicone gel polymer precursors (3.015 g part A+3.015 g part B) were combined with 0.3174 g IRP69-Oct (5% w/w) mixed by hand, poured onto release liner and air bubbles allowed to escape (˜20 min) and cured at 84° C. for 50 min. Cure was complete and not inhibited by the ammonium compound.

### Example 12

### Silicone Rubber IRP69-Ag Polymer Composites

MED-4955 Liquid Silicone Rubber (Nusil Silicone Technology, Carpinteria Calif.) polymer precursors (8 g part A (catalyst prepolymer)+8 g part B (cross-linking prepolymer)) were combined with 0.4948 g IRP69-Ag (3% w/w) and mixed in a speedmixer. The resulting polymer composite mixture was spread on a release liner and placed in a vacuum oven at room temperature to remove air bubbles. Another release liner was placed on top, rolled with ajar to flatten. The composite mixture was cured at 80° C. for approximately 10 min, resulting in a complete, uninhibited cure.

### Example 13

### Silicone Rubber IRP69-Benzalkonium Polymer Composites

MED-4955 Liquid Silicone Rubber polymer precursors (18 g part A+18 g part B) were precombined using a speedmixer. 6.0844 g of the combined MED-4955 components was removed and admixed with 0.3202 g IRP69-BA (5% w/w) by hand, and the resulting polymer composite mixture was spread on a release liner and placed in vacuum oven at room temperature to remove air bubbles. Another release liner was placed on top, rolled with a jar to flatten and then cured (˜80° C., ˜10 min). Cure was complete and not inhibited by the ammonium compound.

### Example 14

### Silicone Rubber IRP69-Cetylpyridinium (CP) Polymer Composites

MED-4955 Liquid Silicone Rubber prepolymers (9 g part A+9 g part B) were precombined using a speedmixer, 6.095 g of this mixture was removed and 0.3208 g IRP69-CP (5% w/w) was combined therewith and mixed by hand. This biologically activated polymer composite mixture was spread on release liner and placed in a vacuum oven at room temperature to remove air bubbles. Another release liner was placed on top, rolled with a jar to flatten and then cured (˜80° C.˜10 min), yielding a completely cured, activated solid polymer composite.

### Example 15

### Silicone Rubber IRP69-Octenidine Polymer Composites

MED-4955 Liquid Silicone Rubber prepolymers (12 g part A+12 g part B) were mixed with a speedmixer and 6.041 g of this prepolymer blend was removed and combined by hand mixing with 0.318 g IRP69-Octenidine (IRP69-Oct) to yield a 5% w/w biologically activated polymer composite. The liquid composite mixture was spread on release liner and placed in a vacuum oven at room temperature to remove air bubbles. Another release liner was placed on top, rolled with ajar to flatten. Curing at 80° C. for 10 min was uninhibited, resulting in a high quality, activated solid polymer composite.

### Example 16

### Silicone Gel IRP69-Cysteamine Polymer Composites

MED 4950 silicone gel (Nusil Technology, Carpinteria Calif.) prepolymers (1.5 g part A+1.5 g part B) were combined with 0.06 g IRP69 cysteamine (2% w/w) using a speedmixer. This biologically activated liquid polymer composite mixture was spread on a polypropylene release liner and then submitted for curing in an oven at 150° C. for 5 minutes. The cure was unimpaired, resulting in a high quality, cured solid Silicone IRP69-Cysteamine polymer composite. Here again, the lack of inhibition of curing observed by the subject biologically active agent, cysteamine, is surprising, because cysteamine is another amino-containing compound predicted to disrupt curing mechanisms in conventional salt forms. More specifically, cysteamine is an amino thiol inhibitor of urease, an enzyme produced by certain bacterial pathogens (e.g., MRSA and Proteus mirabilis).

The instant example illustrates a wide range of utilities, and distinct biological activities, mediated by the novel, biologically activated polymer composites of the invention. Bacterial ureases are distinct targets for biological intervention, apart from direct “antimicrobial” (e.g., bactericidal) activity. In conjunction with various clinical uses, for example the use of urinary catheters, bacterial ureases can cause indirect pathogenic effects on human subjects. In one important context, bacterial ureases break down urea in urine and mediate pH changes that can mediate precipitation of metal salts (e.g., calcium and magnesium phosphate salts) on surfaces or in the environment of urinary catheters. The resulting salt precipitates can ensnare bacteria leading to a substantial increase in detrimental biofilm formation (e.g., by enhancing bacterial adherence/colonization and growth of bacteria on surfaces of urinary catheters), among other adverse consequences. In these and other applications IRP69-cysteamine and other biologically activated polymer composites of the invention can be used to render surfaces and medical devices relatively free of microbial contamination, without exerting strictly “antimicrobial” biological activity (e.g., in the sense of killing bacteria or other microbial targets). In the instant example, the primary biological activity of the IRP69-cysteamine polymer composite is as an enzyme-inhibitory polymer coating or biomaterial. While the end result of employing these coatings and materials may be characterized and quantified as “antimicrobial”, their base activity is to reduce attachment, fouling, colonization and growth of bacteria on medical surfaces, including urinary catheters through anti-urease activity.

### Example 17

### Photocuring Silicone Rubber IRP69-Cysteamine Polymer Composites

Momentive 2060B UV-Curing Liquid Silicone Rubber (Momentive Performance Materials. Albany, N.Y.) polymer precursors (41.9 g part B (crosslinking component), and 1.52 g UV catalyst (photoinitiator)) were preblended in a speedmixer. 7.16 g of this prepolymer blend was removed and combined by hand mixing with 0.3768 g IRP69-cysteamine (yielding a 5% w/w biologically activated liquid polymer composite mixture). The resulting composite mixture was on a release liner and placed in a vacuum oven at room temperature to remove air bubbles. The material was then passed through a UV curing system (Fusion UV Systems, Inc.) at 4 ft/min with each side of the gel exposed to the UV lamp once. In this distinct curing system also, the biologically active agent, cysteamine, was protected or shielded by ionic association within the activated polymer salt particles, so the subject amino compound did not exert inhibition of curing mechanisms (as would be expected for a simple cysteamine salt, e.g., cysteamine hydrochloride).

### Example 18

### Photocuring Silicone Rubber IRP69-Ag Polymer Composites

Momentive 2060B UV-Curing Liquid Silicone Rubber polymer precursors (41.9 g part B (crosslinking component), and 1.52 g UV catalyst (photoinitiator)) were blended with a speedmixer. 7.16 g of this blend was removed and mixed by hand with 0.3768 g IRP69-Ag (5% w/w) liquid, biologically activated polymer composite mixture. This mixture was spread on a release liner and placed in a vacuum oven at room temperature to remove air bubbles. The composite mixture was then passed through a UV curing system (Fusion UV Systems. Inc.) at 4 ft/min with each side of the gel exposed to the UV lamp once. Cure was not inhibited.

### Example 19

### Photocuring Silicone Rubber IRP69-Benzalkonium Polymer Composites

Momentive 2060B UV-Curing Liquid Silicone Rubber polymer precursors (41.9 g part B+1.52 g catalyst) were mixed in a speedmixer. 7.28 g of this prepolymer blend was removed and combined with 0.383 g IRP69-BA (5% w/w) by hand mixing, and the resulting composite mixture was spread on a release liner and placed in a vacuum oven at room temperature to remove air bubbles. The biologically activated composite mixture was run through a UV curing system (Fusion UIV Systems, Inc.) at 4 ft/min with each side of the gel exposed to UV lamp once. Cure was not inhibited.

### Example 20

### Photocuring Silicone Rubber IRP69-Cetylpyridinium Polymer Composites

Momentive 2060B UV-Curing Liquid Silicone Rubber components (41.9 g part B+1.52 g catalyst) were preblended using a speedmixer. 7.11 g of this material was removed and mixed by hand with 0.374 g IRP69-cetylpyridinium (IRP69-CP, yielding a 5% w/w activated polymer composite liquid mixture. This was spread on a release liner and placed in a vacuum oven at room temperature to remove air bubbles. The activated composite was then run through a UV curing system (Fusion UV Systems. Inc.) at 4 ft/min. requiring 4 passes before gel could be removed, followed by one more pass on the reverse side. Cure was lengthy but not inhibited, resulting in a high quality solid, activated polymer composite.

### Example 21

### Photocuring Silicone Rubber IRP69-Octenidine Polymer Composites

Momentive 2060B UV-Curing Liquid Silicone Rubber components (15 g part B+0.544 g catalyst) were mixed by speedmixer, 6.3327 g of the mix was removed and then combined by hand mixing with 0.3333 g IRP69-Octenidine (IRP69-Oct) yielding a 5% w/w polymer composite mixture). This was spread on a release liner and placed in vacuum oven at room temperature to remove air bubbles. The material was run through a UV curing system (Fusion UV Systems, Inc.) at 4 ft/min with each side of gel exposed to UV lamp once, yielding a high quality sold cured composite material.

### Example 22

### Solvent-Based, Polyurethane IRP69-Ag Polymer Composites

Tecophilic Polyurethane (Lubrizol Corporation, Wickliffe, Ohio) prepolymers were combined with a silver (AG)-activated fine particulate polymer salt to form yet another class of solvent-based biologically activated polymer composite. By solvent based is meant that the polymer precursors are dissolved in an organic solvent (in this case chloroform) to dissolve the precursors and render them miscible in a fluid state with the activated fine particulate polymer salt particles. In this example, 3.01 g Tecophilic SP-80A-150 was dissolved in 38.547 mL CHCl₃ on a rollermill. 19.995 g of this solution was removed (containing 1.0 g Tecophilic) and was mixed with 0.0528 g IRP69-Ag by hand mixing. The resulting activated polymer composite mixture was then poured on a release liner to allow the organic solvent (chloroform-CHCl₃) to evaporate. Cure was not inhibited. This example illustrates compatibility of fine particulate activated polymer salts of the invention with organic solvent dissolved polymers. The activated polymer salts are surprisingly stable (e.g., resist dissociation, dissolution, chemical change or degradation) in chloroform and other organic solvents used for dissolving various polymer precursor types that are useful within the invention (including polyurethanes, polyvinyls, polyamides, polyesters, and the like).

### Example 23

### Polyurethane IRP69-Ag Polymer Composites

Tecoflex EG-80A (Lubrizol Corporation. Wickliffe, Ohio) polyurethane polymer composite with IRP69-Ag was constructed as follows. 5.012 g Tecoflex EG-80A was dissolved in 64.209 mL CHCl₃ on rollermill, 20.08 g soln removed (1.0 g Tecoflex) and mixed with 0.0528 g IRP69-Ag by hand, poured on release liner to allow CHCl₃ to evaporate. Cure was not inhibited.

### Example 24

### Silicone Gel IRP64-Ag Polymer Composites

The foregoing examples illustrate aspects and embodiments of the invention employing strong ion-exchangers (exemplified by IRP69-based strong cation-exchangers) to mediate ion-exchange association of biologically active agents within fine particulate biologically activated ion-exchange polymer salts. In the following examples weak ion-exchangers (exemplified by IRP64) are employed. MED 6345 Silicone Gel (Nusil Silicone Technology, Carpinteria Calif.) polymer precursors (12 g part A+12 g part B) were combined by speedmixing with 2.67 g IRP64-Ag, yielding a 10% w/w activated polymer composite mixture. This was poured into mold and air bubbles allowed to escape (˜20 min) and cured at 70° C. for 1 hr. Cure was not inhibited.

### Example 25

### Silicone Gel IRP64-Benzalkonium Polymer Composites

MED 6345 silicone gel polymer precursors (3.07 g part A+3.07 g part B) were combined by hand mixing with 0.3231 g IRP64-BA (5% w/w composite mixture), poured onto release liner, air bubbles allowed to escape (˜20 min) and cured at 84° C. for 23 min. Cure was complete and not inhibited by the ammonium compound.

### Example 26

### Silicone Gel IRP64-Cetylpyridinium (CP)

MED 6345 silicone gel polymer precursors (3.00 g part A+3.00 g part B) were combined by hand mixing with 0.3305 g IRP64-CP (5% w/w composite mixture), poured onto release liner, air bubbles allowed to escape (˜20 min) and cured at 84° C. for 23 min. Cure was complete and not inhibited by the ammonium compound.

### Example 27

### Silicone Gel IRP64-Octenidine

MED 6345 silicone gel polymer precursors (3.060 g part A+3.060 g part B) were combined by hand mixing with 0.3255 g IRP64-BA (5% w/w composite mixture), poured onto release liner, air bubbles allowed to escape (˜20 min) and cured at 84° C. for 50 min. Cure was complete and not inhibited by the ammonium compound.

### Example 28

### Liquid Silicone Rubber IRP64-Ag Polymer Composites

MED-4955 Liquid Silicone Rubber polymer precursors (7.9 g part A+7.9 g part B) were combined by speedmixing with 0.4997 g IRP64-Ag (3% w/w composite mixture), spread on release liner and placed in vacuum oven at room temperature to remove air bubbles. Another release liner was placed on top, rolled with a jar to flatten and then cured (˜80° C., ˜10 min). Cure was complete and uninhibited.

### Example 29

### Liquid Silicone Rubber IRP64-Benzalkonium Polymer Composites

MED-4955 Liquid Silicone Rubber polymer precursors (18.2 g part A+18.2 g part B) were mixed in speedmixer, 6.214 g of the mixture was then removed and combined by hand mixing with 0.3202 g IRP64-BA (5% w/w composite mixture), spread on release liner and placed in vacuum oven at room temperature to remove air bubbles. Another release liner was placed on top, rolled with ajar to flatten and then cured (˜80° C., ˜10 min). Cure was complete and not inhibited by the ammonium compound.

### Example 30

### Liquid Silicone Rubber IRP64-Cetylpyridinium Polymer Composites

MED-4955 Liquid Silicone Rubber polymer precursors (9.1 g part A+9.1 g part B) were mixed in speedmixer, 6.195 g of the prepolymer mixture was then removed and combined with 0.3208 g IRP64-CP (5% w/w) by hand mixing. The composite mixture was spread on release liner and placed in vacuum oven at room temperature to remove air bubbles. Another release liner was placed on top, rolled with a jar to flatten and then cured (˜80° C., ˜10 min). Cure was not inhibited.

### Example 31

### Liquid Silicone Rubber IRP64-Octenidine Polymer Composites

MED-4955 Liquid Silicone Rubber polymer precursors (12 g part A+12 g part B) were mixed in speedmixer. 6.041 g of the prepolymer mixture was then removed and combined with 0.318 g IRP64-Oct (5% w/w) by hand mixing. The composite mixture was spread on release liner and placed in vacuum oven at room temperature to remove air bubbles. Another release liner was placed on top, rolled with ajar to flatten and then cured (˜80° C., ˜10 min). Cure was not inhibited.

### Example 32

### Photocuring Liquid Silicone Rubber IRP64-Ag Polymer Composites

Momentive 2060B UV-Curing Liquid Silicone Rubber polymer precursors (41.9 g part B+1.52 g catalyst) were admixed in speedmixer, 7.16 g removed and 0.3768 g IRP64-Ag (5% w/w) mixed in by hand, the composite mixture spread on release liner and placed in vacuum oven at room temperature to remove air bubbles. The composite mixture was passed through UV curing system (Fusion UV Systems, Inc.) at 4 ft/min, with each side of gel exposed to UV lamp once. Cure was not inhibited.

### Example 33

### Photocuring Liquid Silicone Rubber IRP64-Benzalkonium Polymer Composites

Momentive 2060B UV-Curing Liquid Silicone Rubber polymer precursors (41.9 g part B+1.52 g catalyst) were mixed in a speedmixer, 7.28 g of the mixture was removed and 0.383 g IRP64-BA (5% w/w) was mixed in by hand. The resulting polymer composite mixture was spread on release liner and placed in vacuum oven at room temperature to remove air bubbles. The material was passed through a UV curing system (Fusion UV Systems, Inc.) at 4 ft/min with each side of gel exposed to UV lamp once. Cure was not inhibited.

### Example 34

### Photocuring Liquid Silicone Rubber IRP64-Cetylpyridinium Polymer Composites

Momentive 2060B UV-Curing Liquid Silicone Rubber polymer precursors (41.9 g part B+1.52 g catalyst) were mixed in a speedmixer, 7.11 g of this blend was removed and mixed with 0.374 g IRP64-CP (5% w/w) by hand. The resulting polymer composite mixture was spread on release liner and placed in vacuum oven at room temperature to remove air bubbles. The uncured polymer composite mixture was run through a UV curing system (Fusion UV Systems, Inc.) at 4 ft/min, with 4 passes required before gel could be removed and turned over, then one more pass on the reverse side. Cure was complete.

### Example 35

### Photocuring Liquid Silicone Rubber With IRP64-Octenedine

Momentive 2060B UV-Curing Liquid Silicone Rubber polymer precursors (15 g part B+0.544 g catalyst mixed) were mixed in a speedmixer, and 6.3327 g of this mixture was removed and combined with 0.3333 g IRP64-Oct (5% w/w) by hand mixing. The resulting biologically activated polymer composite mixture was Spread on a release liner and placed in a vacuum oven at room temperature to remove air bubbles. The uncured composite mixture was passed through a UV curing system (Fusion UV Systems, Inc.) at 4 ft/min with each side exposed to UV lamp once. Cure was complete and not inhibited.

### Example 36

### Polyurethane Laquer Polymer Composite With IRP64-Ag

MED-4950 Tecophilic TG-500 Polyurethane prepolymers were combined with IRP64-Ag to form a biologically activated, solvent-based polymer composite. 3.07 g Tecophilic SP-80A-150 dissolved in 38 mL CHCl₃ on a rollermill. Subsequently, 19.975 g of the lacquer was removed from the container (equating to a solids content of 1.0 g Tecophilic TG-500) and the lacquer was combined with 0.0589 g IRP64-Ag and the mixture stirred by hand. The mixture was subsequently dispersed onto release liner and the CHCl₃ allowed to evaporate. The resulting film was durable, cosmetically acceptable, and demonstrated efficacy against several bacteria using a Kirby-Bauer disk diffusion assay.

### Example 37

### Polyurethane Laquer Polymer Composite With IRP64-Ag

5.012 g Tecoflex MG-8020 polymer precursors were dissolved into 64.209 mL CHCl₃ on a rollermill. Subsequently, 20.08 g of the lacquer was removed from the container (equating to a solids content of 1.0 g Tecoflex MG-8020). The lacquer was combined with 0.0528 g IRP64-Ag and the resulting biologically activated, solvent-based polymer composite mixture was stirred by hand. The mixture was subsequently dispersed onto release liner and the CHCl₃ allowed to evaporate. The resulting solid polymer composite film was durable, cosmetically acceptable, and demonstrated efficacy against several bacteria using a Kirby-Bauer disk diffusion assay.

### Example 38

### Preparation of MAC-3-Ag

10.29 g of Dowex MAC-3 (The Dow Chemical Company, Liquid Separations, Midland, Mich. 48641-1206) material was slurried into 150CC of 0.1N NaOH solution for 20 minutes and the MAC-3 filtered rinsed until the filtrate was neutral pH and the solid slurried in 150 cc deionized water and 8.5 g AgNO₃ (Note: 3.8 mEq/g requires 6.64 g to yield 100% of the exchange capacity) was (Fluka) added and the mixture stirred for 1.5 hours in the absence of light. The material was filtered, washed and dried under vacuum at 130° C. and the mass balance determined (13.45 g). This represents an increase in mass of 3.16 grams represents approximately a yield of 56%. The resulting MAC-3 polymer salt thus comprises approximately 56% of the available Na sites on the intermediate form (sodium form) of the resin occupied by silver ion. This provides proof of concept and sufficient guidance for designing a wide range of selectably loaded polymer salts of the invention, with variable loading of active ionic agent(s).

### Example 39

### MED-4955 Liquid Silicone Rubber with MAC-3-Ag

8 g part A+8 g part B+0.5105 g MAC-3-Ag (3% w/w) mixed in speedmixer, spread on release liner and placed in vacuum oven at room temperature to remove air bubbles. Another release liner was placed on top, rolled with ajar to flatten and then cured (˜80° C., ˜10 min). Cure was complete.

### Example 40

### Preparation of MAC-3-Cu and Amberlite IRP64-Cu

Dowex MAC-3 (The Dow Chemical Company, Liquid Separations. Midland, Mich. 48641-1206) or Amberlite IRP64 (Rohm and Haas Company, a subsidiary of Dow Chemical Company. Philadelphia, Pa. 19106-2399) weak cation-exchange material (Na+ form) was stirred in a minimal amount of deionized water and a large excess (˜10-500 molar excess) of copper sulfate (Cu(SO₄)₂) and the mixture stirred for 1 hour at room temperature using a mechanical stirrer. The (blue-colored) salt was filtered and rinsed with deionized water until the filtrate was clear (no blue). The salt was dried at 130° C. under vacuum and used in the formulation of silicone rubber materials. Neither MAC-3-Cu nor Amberlite IRP64-Cu inhibited the cure of silicone elastomers (Momentive Performance Materials UV-curing silicone (2060) or Nusil MED-4955.

### Example 41

### Preparation of Polymer Salts Using Crosslinked Polycarboxylated Weak Cation-Exchange Materials

Amberlite IRP64 and MAC-3 weak cation-exchange material was stirred in a minimal amount of deionized water and a large excess (˜10-500 molar excess) of the salt (containing the biologically active exchange cation of interest, such as benzalkonium chloride) was added and the mixture stirred by the addition of a mechanical stirrer for 60 minutes. The solid was filtered washed with copious amounts of deionized water (until the filtrate does not contain any of the active ionic agent (benzalkonium chloride for example) as evidence from ultraviolet spectroscopic evaluation of the filtrate. The modified IRP64/MAC-3 was dried under vacuum at 130° C. and the material was milled with the aid of an IKA homogenizer and the milled particulate polymer salt put through a sieve with a 35 μm cutoff. The powder was dried under vacuum and used for addition to various polymer composite formulations.

### Example 42

### Preparation of Amberlyst A21-Acetylsalicylic acid (ASA)

Amberlyst A21 (4.6 mEq/g exchange capacity) and acetyl salicylic acid (ASA) were stirred together at room temperature in a solution of water/isopropyl alcohol (2:1) for 12 hours and the product filtered, washed with water and soxhlet extracted with isopropyl alcohol (12 hours). The product (Amberlyst A21-ASA) was air and vacuum dried, and the yield determined to be 30% of theoretical exchange capacity) milled to 5 μm particle size and incorporated into LSR silicone rubbers Performance Materials UV-curing silicone (2060) and Nusil MED-4955 (UV curing and thermal curing materials respectively). Both materials cured as expected and the release of ASA from the silicone materials was observed and monitored by UV spectroscopy with exposure of the material to PBS solution.

Strong and weak cation exchange resins can be substituted with ions by one of two methods. These include: 1. Protonation of an available group that can accept a proton, e.g. a nitrogen moiety such as that found on the free base of tetracycline, or 2, by ion exchange, i.e. the placement of silver in exchange for sodium, or the placement of chlorhexidine diction for two metal cations (Na+). Thus, a resin can be made to house at least one cation and may be functionalized to contain two or more ions. As an example, a strong cation exchange resin (in acid form with an exchange capacity of 4.5 mEq/gram) cane reacted with 1.5 mEq/gram of tetracycline free base and subsequently be reacted with 3.0 meQ/gram of silver acetate (AgOAc). The result of this set of reactions is a mixed cation salt that contains 1.5/4.5 tetracycline and 3.0/4.5 Ag. Other examples can include a weak cation exchange resin (exchange capacity of 10 mEq/gram), potassium form that is first reacted with (exchanged with) tobramycin hydrochloride (3.0 meQ/gram) and 3.0 mEq/gram minocycline hydrochloride. Thus the mixed tobramycin-minocycline product occupies 6.0 mEq/gram and the remaining 4.0 mEq/gram remains potassium. In yet another example, the strong cation exchange resin of the first description can be modified to include copper, zinc, and sodium by the addition of copper (II) acetate (2.0 mEq/Gram, keeping in mind that Cu(II) is divalent and thus requires only 1.0 mEq/gram to account for this. Similarly, zinc (II) acetate is added at 2.0 mEq/gram (same as for copper (II)) and the remaining 0.5 mEq/dram is added as sodium acetate. In yet another example, a strong anion exchange resin (chloride form) is reacted with dexamethasone sodium phosphate (⅓ of available sites) and acetylsalicylic acid, sodium (⅔ of available sites). This resin may be back titrated to remove chloride by the addition of sodium acetate if desired.

For resins that incorporate organic molecules (tobramycin, chlorhexidine, minocycline, tetracycline), the active drugs are dissociable from the resin in the presence of ionic media such as urine, blood, saliva. Thus with the incorporation of the resin into a polymer matrix, the release of the active species (e.g. tetracycline) has been measured using a UV spectroscopic method. The kinetics are first order (diffusion controlled) and the amounts released can be measured against a standard curve created for tetracycline hydrochloride. The diffusion of the drug from the matrix is also verified using a Kirby Bauer disk diffusion assay. In a more expansive example, a silicone tubing created to include IRP69-Ag is over-molded to include a buttress of silicone containing IRP64-Chlorhexidine. Thus the device has the ability to release Ag+ and chlorhexidine (likely as the chloride).

Loading of biologically active agents, and controllable release kinetics of these agents under selected conditions (e.g., upon being placed in contact with ionic solutions, including plasma, wound fluid, saline, urine, etc.) can be uniquely and powerfully controlled, varied and selected during construction and use of the biologically activated fine particulate polymer salts of the invention, for distinct uses and purposes, according to the teachings herein. Yet another previously unrecognized problem confronted and resolved here relates to the practical utility of activated polymer salts comprising ionic biologically active agents for incorporation into curable, polymerizing activated polymer composites. During the development of the synthetic procedures designed to load ions onto the strong cation exchange resin backbone, it was observed that drying the acid form of the resin, and also removal of water from the resin via dehydration by washing with partially-aqueous or non-aqueous solvents, such as ethanol, led to chemical reactions that were not desirable. For example, exposure of the acid form of a strong cation exchange resin (—SO3H), a strong odor of an organic solvent-like residue was noted.

By extensive investigation it has been further discovered that that certain resins employed in construction in some way promotes unexpected formation of ether compounds, for example diethyl ethers. When isopropanol is used in conjunction with ethanol and methanol, mixed ethers were evidently formed. Because the subject resins are strong acids, ethers can be formed by protonation and subsequent dehydration. It was also determined with this process that the acid forms of resins were altered from the required form for preparation of polymer composites. With these subtle, yet clear transformations of the subject resins, subsequent exchange to form a strong cation exchange salt, such as IRP69-Cu, results in a copper salt that catalyzes or otherwise promotes degradation of a variety of polymer matrix materials it was incorporated into. These intolerant polymer systems included silicones (Q7-4750, 2.0 wt % IRP69-Cu) and polyurethanes, such as Tecothane 80A. Strong cation exchange resins treated with alcohols while in their respective acid forms, consistently affected the matrices of which they were later incorporated into. As such, it was unexpectedly determined to be important to avoid attempts to dehydrate the polymer salts during and after derivatization/activation with ionic active agent, either chemically or by the use of heat. With heat, the sulfonic acid moieties of desired resins are labile, thus resulting in formation of sulfuric acid (SO3) and possibly other undesired reaction products.

Following thermal treatment of the sulfonic acid forms of exemplary resins, acid content was measured in aqueous extracts (wash solution) of these resins, as determined by reduced pH. Optimal procedures developed here specifically avoid use of alcohols and heat. This requirement led to a further discovery of tools, methods and mechanisms allowing for ready calibration, loading and titration of the exchange capacity of ion-exchange resins for adjusting levels and parameters of their loading with biologically active ionic agents. In conjunction with these principles, the methods herein were refined and elaborated to include an optional titration protocol, for example using first sodium sulfate to remove protons from a subject acid [e.g., R-SO3H+Na2SO4→R-SO3-Na++H [symbol] SO4Na] and subsequently titrating the sodium hydrogen sulfate using a standardized solution of sodium hydroxide. This procedure, intended for one newly-appreciated purpose, screndipitously led to discovery of a convenient, powerful method for determining and calibrating/selecting exchange capacity of wet resins for activation, precluding the need to dry a resin prior to an exchange reaction. Subsequently, it was determined that with reaction of the resin (acid form) with a metal acetate or a free organic base (amine), such as tetracycline base, that some of the sulfonic acid residues remained un-exchanged. In other words, sulfonic acid remained on the resin backbone. We determined that in these cases it was possible to back-titrate with sodium acetate (although we could also use silver acetate etc.) to subsequently ensure that no residual sulfonic acid remained and that this back titration could be accomplished without removing any (or in some cases only small amounts) of the bound ion (tetracycline). With this back titration we determined that we could thermally dry the resulting resin without the complication of degradation. This back titration works flawlessly for a variety of ions that do not achieve 100% incorporation. Two examples include tetracycline and chlorhexidine.

It is particularly worth noting that the use of acetate and other carboxylic acid salts is particularly useful for pairing with the acid form of strong cation exchangers. The reason for this can be rationalized using a strong acid (SO3H) titration with a weak base (—OAc) to yield a weak acid (HOAc) plus a salt.

Copper salts are known to have antimicrobial properties, i.e. bactericidal, virucidal, and fungicidal. However, copper salts have not found application in paints and coatings particularly for use in the indoor environment. For the outdoor environment, there are some solutions that include copper (II) pyrithione and zinc (II) pyrithione. It is important to note that both of these compounds have appreciable toxicity and cannot be used indoors and as such they are not recommended for use in decorative paints such as low VOC acrylic latex enamels. Furthermore, it is important to note that copper (II) and zinc (II) compounds can interact with surfactants, such as dodecyl sulfate and other ionic surfactants to result in precipitation, aggregate formation, i.e. congealing, solidification, or at a minimum changes in viscosity to aqueous paint formulations. In the event that a copper (II) or Zn (II) salt could be introduced to an aqueous paint formulation without affecting the bulk properties of the formulation, there would remain a concerns that: the salt could leach from the paint formulation to result in discoloration, the salt could degrade the formulation or prevent cure, and or lead to overt toxicity to those exposed to the painted surface or the paint formulation. In our first (in paint) evaluation, a Dunn Edwards brand paint was mixed to include IRP69-Cu and in a separate experiment. IRP64-Cu. Within a short period of time, aggregation was observed for both. When the same paint was treated with an identical amount of copper sulfate. The paint cured into a solid block within minutes. When the additives and copper sulfate was added to a Glidden decorative acrylic latex enamel, no aggregation was observed, however a change in viscosity was noted making it difficult to apply consistent coatings using a brush.

In order to alleviate this problem observed with the addition of IRP69-Cu and IRP64-Cu, i.e. precipitation, increased viscosity etc., we determined that we could alleviate the Lewis Acid nature of copper by satisfying its empty orbitals by the addition of a Lewis Base (ammonia, NH3). As such, ammonia adds to the green copper (II) salts of IRP69 and IRP64 to forma dark blue complex that can reversibly give up ammonia with heating or drying. Thus the addition of the ammonia (Lewis base)-resin (IRP64/IRP69-Cu) (Lewis acid) complex to the decorative paint to include the Dunn Edwards and Glidden examples DID NOT result in aggregation, precipitation, or viscosity changes to either of the paint formulations at a variety of concentrations. We note that this observed metal amine complex formation is expected to work for the zinc (II) salt as well. This is of importance because zinc (II) is known to possess antimicrobial and antifouling properties and it will interact equally (as bad as) Cu(II) in paints and coatings containing surfactants. As such, the metal-ammonia complex provides a broad-reaching solution.

It is worth noting that this approach to adding copper derivatives to paints may translate to other copper (II) examples. Furthermore

Although copper salts are generally understood to possess toxicity, the copper complexes of strong and weak cation exchange resins are much less toxic than many of their counterparts used today, i.e. copper pyrithione or

Similarly ASA was associated with the strongly basic anion-exchange resin AMBERLITE FPA40-CI (exchange capacity >1 mEq/g), a food grade strong base anion-exchanger (a polyamine/polyammonium salt0. This resin, a polyaminated ion-exchange material, demonstrated effective binding (˜1.0 mEq/g) of ASA, and similarly binding and releasing dexamethasone sodium phosphate (DexSP) anion.

Under mild conditions at room temperature in aqueous ethanol, ASA can be bound to Amberlyst A21 to yield an ion-exchange material including about 30% of the theoretical exchange capacity of the material (4.6 mEq/gram). Following soxhlet extraction (isopropanol) and drying, the material was incorporated into Nusil MED 4950 (NuSil Technology LLC, 1050 Cindy Lane, Carpinteria, Calif. 93013) and the cure of the silicone proceeded uninhibited. ASA is readily released from the resulting composition intact, as determined by UV spectroscopy. ASA cannot be incorporated as the free acid as it is not stable once heated in the curing process. This example is another representation of the stability imparted as a consequence of integrating an organic molecule with an ion-exchange backbone.

### Example 43

### Incorporation of Amberlyst A21-ASA Into Silicone Rubber

Amberlyst A21-ASA was successfully incorporated into Momentive Performance Materials 2060 UV-curing LSR without any inhibition of cure.

### Example 44

### Incorporation of Chlorhexidine Into Silicone Rubber

Chlorhexidine (CHX), a molecule that is susceptible to thermal degradation to yield the carcinogen, p-chloroaniline, above 70° C. is stabilized when bound to the ion-exchange material (polystyrene sulfonate (PSS) as well the crosslinked version IRP69). In binding the active antiseptic to an ion-exchange material (IRP64) using the reaction of IRP64-H (acid form) with chlorhexidine diacetate. The yield of IRP64-chlorhexidine is approximately 80% of the theoretical exchange capacity of 10 mEq/g (5 mEq/g for a dication such as chlorhexidine). Once the resin is thoroughly dried, it can be readily milled to submicron size where within the jar the temperature can reach approximately 80° C. Under these circumstances, in the presence of water, the risk of hydrolysis to yield p-chloroaniline increases substantially.

### Example 45

### Octenidine Hydrochloride

Octenidine hydrochloride has been observed to inhibit the cure of thermal and UV-curing silicone rubber materials at levels of less than 2 wt % loading. As such it is of little utility to be included into a silicone material. In addition to the inhibition of cure, such an approach can lead to porosity once the compound has eluted from its matrix. However, the binding of octenidine to IRP64 by the reaction of IRP64-Na with octenidine dihydrochloride. In this reaction, approximately 3.0 mEq/g of the dicationic octenidine (6.0 mEq of IRP64 sites). The material was milled to 1-10 micron particle size and incorporated into silicone rubber as high as 5.0 wt. % without any inhibition of thermal and UV curing silicone rubber.

### Example 46

### Preparation of Copper Cellulose Phosphate

Copper cellulose phosphate can be prepared by exposing sodium cellulose phosphate to an excess of copper (II) sulfate in deionized water, filtering and washing the solid until no residual copper (II) sulfate is detected in the filtrate. Similarly, cellulose phosphate (acid form) can be used in conjunction with copper (II) acetate to yield the cellulose phosphate copper salt and acetic acid. Cellulose phosphate materials derivatized to include metal ions such as copper may be provided as additives for the manufacture of articles to include drywall construction material for example.

Strong and weak cation-exchange resins modified to incorporate Cu(II) can be incorporated into polymeric materials to render the surfaces effective against bacteria, viruses, and fungi for example. In the case of a polymer matrix surface, to include laminate materials, incorporating Cu (II) or Fe(II) modified ion-exchange resins, the use of hydrogen peroxide solution with or without HEPES buffer (Fenton or modified-Fenton reaction) can be used to aid in the disinfection of such surfaces. The addition of peroxide to surfaces comprising metal ion-modified ion-exchange resins can result in the formation of free radical species that can be efficient at killing microbial pathogens.

### Example 47

### Kirby-Bauer Zones of Inhibition

Experiments that have evaluated the antimicrobial capability of Dow Chemical Dowex MAC-3 (The Dow Chemical Company, Midland, Mich.) weak cation-exchange material and Amberlite IRP64 (Rohm and Haas Co., Philadelphia. Pa.) weak cation-exchange material in Cu(II) and Ag(I) forms were shown to have significant zones of inhibition in Kirby-Bauer assays and the zones were determined to be similar to those of strong cation-exchange materials such as Amberlite IRP69 modified to include the same cations of (Cu(II) and Ag(I)) according to the methods of the invention, when tested against Staphylococcus epidermidis, Staphylococcus aureus, Pseudomonas aeruginosa, and Enterococcus faecalis.

### Example 48

### Incorporation of Activated Ion-Exchange Polymer Salts With Silicone

Dowex Mac-3 and IRP64 weak cation (polycarboxylate) exchange resins were modified to include silver ion (See, for example published US patent application No. US20100247544A1 entitled “Compositions and Methods for Promoting the Healing of Tissue of Multicellular Organisms” and published Sep. 30, 2010, the entirety of which is incorporated by reference herein). This silver activated Mac-3 can be dried under vacuum (135° C.) to yield an off white solid that was ground to particles and sieved with a 35 μm cutoff sieve. The particles can be dried again under vacuum and formulated into two silicone materials and these materials (silicones with Ag-Mac-3 and the Ag-Mac-3 alone as particles) were evaluated using a Kirby-Bauer assay. Amberlite IRP64 was treated with 0.1N NaOH solution and the sodium salt (Amberlite IRP-64 (Na+)) was filtered and washed with deionized water until the pH of the filtrate was neutral. The salt is used in alternate examples to prepare Ag+, Cu++, benzalkonium+, chlorhexidine++, octenidine++, doxycyline+, minocycline+, as well as mixed ion material salts, such as materials incorporating silver and copper, silver and zinc, or copper and zinc ions simultaneously. As salts, these particles were incorporated into LSR silicone rubber materials at 5 and 10% loading w/w. Silicones and polyurethanes including various additives comprising a variety of metal and organic ions have been prepared at loading of up to 50 wt %. It is feasible to incorporate the activated fine particulate polymer salts as additives in composite mixtures as described herein at levels greater than 25 wt %, however loadings between 1 wt % to 10 wt % appear to be highly active for most materials and uses.

### Example 49

### Platinum Catalyzed Silicone Rubber with Ammonium Salts

Previous attempts to cure platinum catalyzed silicone rubber formulations compounded with benzalkonium chloride, cetyl pyridinium chloride and other ammonium salts have met with failure. The two-part rubber remained liquid-like of petrolatum consistency. In addition, when curing thermal silicone systems, temperatures of 150oC are required for periods of generally at least five minutes. Because benzalkonium chloride melts at 35° C., the salt, if dispersed as a particle will become molten during the attempt to cure the polymer thus causing the compound to flow/ooze from the material. However, upon exchange of chloride for polysulfonated anions such as for strong cation-exchange materials including non-crosslinked polystyrene sulfonate and crosslinked polystyrene sulfonate such as Amberlite IRP69 or crosslinked carboxylated weak cation-exchangers such as Amberlite IRP64, the silicone rubber materials (e.g., Nusil MED 4950 thermal curing system and UV curing system, Momentive Performance Materials 2060 UV-curing liquid silicone rubber or LSR), demonstrated full cure under normal curing conditions without any melting of the added material and resulted in the absence of any voids in the material. This unexpected result provides for the preparation of silicone rubber materials that demonstrate zones of inhibition against bacterial species amenable for medical uses to combat bacterial infection and surface transmission. The tables and other description herein demonstrate the effectiveness of a fully representative array of biologically activated polymer salts of the invention 5% Amberlite IRP69-Benzalkonium (Amb-BA), Amberlite IRP69-cetyl pyridinium (Amb-CP), and linear (water-soluble) polystyrene sulfonate salts of benzalkonium (PSS-BA) and cetylpyridinium (PSS-CP) composited with silicone rubbers (Nusil MED 4955 and Momentive 2060 LSR) against Staphylococcus aureus and Enterococcus faecalis. This demonstrates activation of silicon polymer composites using ammonium polymer salts using a (platinum) curing silicone system with a non-crosslinked strong cation-exchanger (PSS), further expanding the compositions and uses provided here for constructing ion-exchange polymer salt composites with silicone and other thermoset, thermoplastic and photocuring polymers. These various exemplary composites were also effective against Staphylococcus epidermidis and to a lesser degree against the Gram-negative Pseudomonas aeruginosa.

### Example 50

### Size Characterization of Activated Polymer Salt Particles

Size reduction of the initial resin materials used herein (e.g., commercially available Rohm & Haas resin, IRP69-Na) has been tested and optimized for various contemplated uses. In one example a starting resin material was milled through two consecutive milling steps where, A) is Poly(Sulfonated Styrene-divinylbenzene) IRP69-Na (Rohm & Haas) as received, B) post milling with 5 mm stainless steel media in heptane non-solvent, C) post milling with 0.5 mm or smaller zirconia media in heptane non-solvent. With each milling step the size distribution becomes more refined around the median value. As received, IRP69-Na size distribution spans approximately 10-1400 μm (Δ=1300 μm). Following the first milling step the object resin particle size range spanned approximately 0.8-10 μm (Δ=9.2 μm), and after the second milling step the distribution spanned approximately 0.1-0.8 μm (Δ=0.7 μm). The first milling step utilized stainless steel and the non-solvent medium heptane and in the second milling step zirconia ceramic was utilized with heptane non-solvent.

Each of the above-described, antimicrobial or antifouling polymer composites exhibit high levels of antimicrobial or other anti-biologic activity, according to the various assays for bactericidal and bacteriostatic activity, inhibition of bacterial transfer contamination risk, and other anti-biologic target activities, including marine anti-fouling, as described herein. Further surprising studies reveal that the novel composites of the invention, incorporated in coatings, biomaterials or medical devices, present greatly reduced cytotoxic or other harmful impacts on healthy mammalian cells and tissues exposed to the activated composites (e.g., compared to simple salts of the same active agents incorporated in the composites of the invention).

Table 7 below depicts cytotoxicity results from an MTS assay vs. following direct contact exposure of antimicrobial IRP69-modified silicone rubber (Q7-4750) with confluent human neonatal fibroblasts in culture for a period of 5-days. The control sample, silicone rubber, is known to be very biocompatible and all readings were normalized to this control (100%). IRP69 is the poly(sulfonated-styrene-divinylbenzene) copolymer where Ag=silver, Cu=copper, BA=benzalkonium. These data reveal that the samples are surprisingly highly compatible with fibroblasts, with 81% of cells remaining viable for the IRP69-benzalkonium-modified silicone and 93% remaining viable for the IRP69-Ag-modified silicone rubber (whereas negative control (AgNO3)-modified silicone exhibited 0% viability following a five day period of direct contact).

TABLE 7
MTS Assay results for human neonatal fibroblasts in culture exposed
to GARDION ™ BIOCIDE-modified silicone rubber. Results
are presented as absorbance (495 nm) and corresponding % viability.

| | Absorbance | |
|---|---|---|
| Sample Material | (495 nm) | % Viability |
| Silicone Control | 1.95 | 100 |
| 5.0 wt % AgNO3 in silicone | 0.01 | 0 |
| 2.0 wt % IRP69-Ag in silicone rubber | 1.81 | 93 |
| 1.0 wt % IRP69-Ag/1.Qwl % ltlP69-Cu in | 1.84 | 94 |
| silicone rubber | | |
| 2.0 wt % IRP69-Cu in silicone rubber | 1.87 | 96 |
| 2.0 wt % IRP69′BAin silicone rubber | 1.58 | 81 |

Table 8 below depicts cytotoxicity results from an MTS assay vs. following direct contact exposure of antimicrobial IRP69-modified silicone rubber (Q7-4750) with confluent human epithelial cells in culture for a period of 5-days. The control sample, silicone rubber, is known to be very biocompatible and all readings were normalized to this control (100%). IRP69 is the poly(sulfonated-styrene-divinylbenzene) copolymer where Ag=silver. Cu=copper, BA=benzalkonium.

TABLE 8
Table 7 - MTS Assay results for primary human epithelial
cells in culture exposed to GARDION ™ BIOCIDE-
modified silicone rubber. Results are presented as
absorbance (495 nm) and corresponding % viability.

| | no | Blank | BA | Cu | Ag/Cu | Ag |
|---|---|---|---|---|---|---|
| | silicone | silicone | (2%) | (2%) | (1%/1%) | (2%) |
| Absorbance | 0.9435 | 0.8125 | 0.089 | 0.4118 | 0.47175 | 0.622 |
| @ 490 | | | | | | |
| % of Blank | 116% | 100% | 11% | 51% | 58% | 76% |

### Example 51

### Analysis of Surface Characteristics and Bacterial Adhesion to Extruded Silicone Rod Incorporating 2 wt % IRP69-Ag

SEM analyses have shown extruded Dow Corning Q7-4750 (rod form) formulated to include 2 wt % IRP69-Ag silicone does not possess overly activated surfaces as determined by a bacterial adhesion assay and subsequent image analysis of the surfaces. Field emission scanning electron micrographs (FE-SEM) of silicone rod samples containing IR69F-Ag (500 nm mean particle size) at 2% w/w; showed no surface irregularities after being challenged with 10⁵ CFU of E. coli for 24 hours or 4 hours with 10⁸ CFU of S. aureus. Following exposure a small number of bacterial colonies are observed on the surface.

### Example 52

### Adhesion Assay

Silicone rubber samples of Dow Corning Q7-4750 were formulated to include 2 wt % IRP69 modified with Copper (Cu), Benzalkonium, Silver (Ag), and Ag/Cu, as well as a blank unmodified silicone sample following exposure to 10⁸ CFUs of E. coli and cultured in tryptic soy broth at 37° C. for 18 hours at which time the samples were lightly rinsed with phosphate buffered saline (PBS) PBS to remove the loosely adhered bacteria and subsequently sonicated in PBS to remove adherent cells. Serial dilutions of the sonicated samples were made prior to plating on standard plate count agar. The resulting data revealed that the modified surfaces showed no significant reduction in bacterial adhesion when compared to controls.

### Example 53

### Time-to-Kill Assay: IRP69-Ag-Modified Silicone Rubber (2.0 wt %) vs. Staphlococcus Aureus

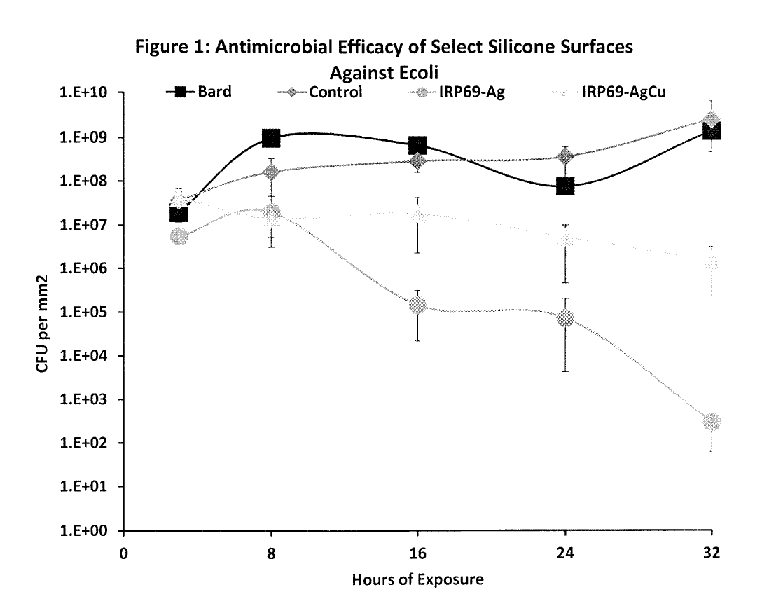

As shown in FIG. 1, following an inoculum of 10⁸ CFU/mL of E. coli in synthetic urine (recipe), samples were incubated at 37° C. and at time points of 0, 3, 8, 16, 24, and 32 hours samples were removed from test and adherent bacteria removed and counts determined. The data reveal that after 3 hours a one-log reduction is observed and after 32 hours a 6-log reduction is evident. These data indicate that activated polymer composite surfaces of the invention will significantly reduce bacterial colonization of the inner lumen of urinary catheters, particularly where protein accumulation on catheter surfaces is low or absent.

### Example 54

### Antimicrobial Sulfonated Polystyrene-Co-Divinylbenzene (IRP 69)

Exemplary silicone materials (e.g., Q7-4750) composited with 2.0 wt % of IRP69-Ag, IRP69-benzalkonium, IRP69-Cu, and binary formulations to include 1.0 wt % of each of IRP69-Cu/IRP69-benzalkonium and IRP69-Ag/IRP69-benzalkonium, were shown to be highly effective at reducing surface bacterial counts, even after pretreatment of the surfaces with fetal bovine serum (Table 8). Table_{[DW4]} 9 below demonstrates bacterial log reduction results using a modified ASTM E2180 (ASTM International, West Conshohocken, Pa., 2007) assay, following inoculum of 10⁶ relevant pathogens-tested against sulfonated polystyrene-co-divinylbenzene (IRP69)-modified MED-4950 silicone rubber polymer complexes comprising activated polymer salts of the invention incorporating a diverse array of biocides, loaded at varying concentrations.

TABLE 9
Table 9 - Log reduction results for GARDION ™ BIOCIDE-
modified MEP-4950

| | Staphylococcus | Escherichia |
|---|---|---|
| Biocide | aureus | coli |
| IRP64-CHX (2 wt %) | 5.63 | 0 |
| IRP69-Ag/IRP64-CHX (1:1, 2.0 | 5.63 | 5.66 |
| wt %) | | |
| IRP69-Minocycline (2.0 wt %) | 1.64 | 0 |
| IRP69-Cu (2.0 wt %) | 6.09 | 0.98 |
| IRP69-Ag/IRP69-Mino (1:1) | 6.09 | 6.38 |
| (2.0 wt %) | | |
| Ag/CHX (1:1) (4.0 wt %) | 6.09 | 6.38 |
| Ag/BA (1:1) (4.0 wt %) | 6.09 | 6.38 |

Table 10 below demonstrates percent bacterial reduction results of a modified ASTM E2180 (ASTM International, West Conshohocken, Pa. 2007) assay using an inoculum of 10⁶ E. coli for Ag-Sulfonated polystyrene-co-divinylbenzene modified (IRP69-Ag) Q7-4750 silicone rubber to include Ag (2.0 wt. %) compared to an non-modified control silicone (Q7-4750). These data reveal that exemplary, IRP69-Ag-modified silicones of the invention are capable of killing up to 95-100% of an inoculum, even after exposure to 10% fetal bovine serum (FBS) (indicating protein adsorption imposes little or no activity reduction, correlated with results using control silicone rubber).

TABLE 10
Table 10-% bacterial reduction for silicone (Q7-4750)
modified to include GARDION ™ BIOCIDES

| | % Efficiency of |
|---|---|
| Biocide | Biocide |
| Control (NoFBS) | 0 |
| Control FBS | 16.0947 |
| SCE-Ag_No FBS | 99.9995 |
| SCE-Ag_with | 99.9995 |
| FBS | |

Table 11 below details the bacterial log reductions from a modified ASTM E2180 assay (ASTM International, West Conshohocken, Pa., 2007) utilizing Staphylococcus aureus against modified MED 4950 (Nusil Technology, Carpinteria, Calif.) silicone rubber modified to include Sulfonated polystyrene-co-divinylbenzene (IRP69) including varying concentrations by weight of IRP69-Ag (0.25-5.0 wt. %) following extraction in PBS at 37° C. The modification of the method involves using a small-pore mesh made out of polypropylene to evenly distribute an agar slurry (0.01 M PBS, 0.0033 w/v % agar) inoculated with 10⁵ Staphylococcus aureus onto the surface at intervals before and after extraction. The assay reveals that after four weeks of extraction in 0.01 M PBS the silicones modified with 1.0, 2.0, and 5.0% IRP69-Ag yielded reductions of 6-logs against the bacterium.

TABLE 11
Table 11 - Bacterial log reductions from inoculated MED-4950
silicones including GARDION ™ BIOCIDES

| IRP69-Ag (wt %) in | | | | | |
|---|---|---|---|---|---|
| Nusil Technology | Organism = Staphylococcus aureus | | | | |
| MED 4950 silicone | Week 0 | Week 1 | Week 2 | Week 3 | Week 4 |
| 5% | 6.45 | 5.85 | 6.56 | 6.48 | 6.48 |
| 2% | 6.45 | 5.85 | 6.56 | 6.48 | 6.48 |
| 1% | 6.45 | 5.85 | 6.56 | 6.48 | 6.48 |
| 0.50% | 5.02 | 5.85 | 6.56 | 6.48 | 6.48 |
| 0.25% | 4.38 | 3.58 | | | |

### Example 55

### Milling of Ion-Exchange Materials for Construction of Fine Particulate, Activated Polymer Salts

One exemplary high energy milling process for use within the invention utilizes planetary ball milling in a ceramic (zirconium) lined stainless steel milling vessel. Zirconia milling media (3.0 mm) are added into the chamber to occupy approximately ⅔ of the bulk chamber volume. Approximately ⅓ of the bulk volume is occupied by any of the porous activated ion-exchange polymer salt particulate material. A non-solvent liquid is then added in an amount approximately equal to ⅓ of the container bulk volume (typically the non-solvent is added so as to percolate into and fill void spaces between milling media and activated polymer salt particles, and to fill void, pore and channel spaces within the porous polymer salt particles. The non-solvent liquid may comprise a heptane non-solvent, or any other suitable non-solvent. Suitable non-solvents more generally can include, for example, intermediate or high boiling point alkanes, exemplified by heptane or mixtures of heptanes, octane, isooctane (2,2,4-trimethylpentane), petroleum distillates (high boiling Pet ether). Lower boiling solvents such as hexane can be used, however this may raise the risk of fire or explosion.

Within the instant example, the milling vessel was topped off with non-solvent, sealed then placed (clamped) into a PM100CM planetary ball mill. The sample milled for approximately 2 hours at 500 rpm. After this milling was stopped (more generally, when a desired milled particle size and uniformity are obtained), the fine particulate ion-exchange polymer salt is separated from the non-solvent (e.g., by evaporation) and media (e.g., by sieving).

In other working examples, a second stage of milling was conducted, wherein the activated ion-exchange polymer salt particles were second stage-milled using smaller zirconia milling media (0.5 mm). According to this exemplary two stage milling process, particle size (alternatively expressed as average or median diameter) and size variation for the fine particulate biologically activated ion-exchange polymer salt materials were shown to be within a predicted, desired size range and to have a predicted, desired particle size uniformity. Briefly, particles from the second stage of milling exhibited particle sizes and uniformity measured at approximately 500 nm average diameter with standard deviations of approximately ±0.75 μm, in other examples approximately ±0.50 μm, and in other examples about ±0.25 μm.

Temperature of milling is an optional control condition that can yield improved milling results in certain embodiments. In demonstration milling runs, excellent milling results were obtained as described above when the temperature of the milling vessel and contents was maintained, for at least a portion of a milling cycle (measured using an IR thermometer), at approximately 80-85° C. This elevated, controlled temperature imparted to the milling chamber and contents elevates pressure within the sealed vessel chamber and lowers viscosity if the milling milieu (non-solvent, milling media and activated ion-exchange polymer salt material) improved milling outcomes for some samples compared to results observed at lower milling temperatures.

Alternative milling methods useful within the invention include hammer milling, which can be employed in the first milling step in order to generated particles with sizes of 1-10 microns. This has been demonstrated here using a Hosakowa hammer mill. Similarly, jet milling may also be of value in this first step. Both hammer milling and jet milling will alleviate the need to use non-solvent milling techniques in all steps in order to provide particles of the optimal size. It is also worth noting that nanoparticulate materials are likely not needed for all applications. In fact, for building materials the incorporation of particles of approximately 10 microns will be acceptable in most cases. Where finer particulates are required, i.e. medical devices and prosthetics for example, a secondary reduction using planetary milling will be required. However, planetary milling may be vertical or horizontal in equipment much larger than in the instant case of jar-based batch methods.

### Example 56

### Surface Characteristics and Stability of Activated and Partially Discharged and Environmentally Exposed Polymer Composites

The use of fine particulate activated ion-exchange polymer salt materials in formation of composites (by combining the activated polymer salt particles with polymer precursors to form solid composites) yields polymer composites of the invention having surprisingly uniform and smooth surface properties free of voids and cracks or other surface defects and absent voids following extraction with ionic (PBS) media. Additionally, these biologically activated polymer composites retain their distinctly smooth and unmarred surface character even after exposure to aging and exposure to photodegradative, thermal degradative, microbial degradative, and chemically transforming (e.g., ionizing, oxidizing, hydrolyzing) environmental conditions. Demonstrating exemplary surface characteristics and performance of these inventive composites, field emission scanning electron micrographic (SEM) images of silicone rod samples containing 0.5 micron (average) diameter, milled IR69F-Ag at 2.0% silver loading (w/w) before and after PBS and saline extraction, magnification 2000× and Silicone rod samples containing IR69F-Ag at 2% (w/w), magnification 7000× revealed pristine surfaces and unexpectedly without any evidence of porosity following ion-exchange. These samples were challenged, in 24-well plate format, with 1.0 mL of 10⁵ CFU/mL of E. coli (Panel C) added to a well containing 1.0 mL of tryptic soy broth and the samples exposed for 24 hours at 37° C. In similar Fashion, a sample (Panel D) was challenged, in 24-well plate format, with 1.0 mL of 10⁸ CFU/mL of E

S. Aureus added to a well containing 1.0 mL of tryptic soy broth and the samples exposed for 24 hours at 37° C. In these examples, the samples were exposed to microbial, and ionic factors that may be expected for the most challenging, practical microbial contamination conditions for a product to experience. As revealed by our SEM images of 0.5 micron particle size IR69F-Ag-modified silicone slab were pristine with and without PBS or saline extraction and images of tubing modified to include 0.5 micron particle size IR69F-Ag at 2.0 wt % with exposure to 10⁵ CFUs of E. coli and after 10⁸ CFUs S. Aureus revealed only minor bacterial contamination. Study samples were removed from the wells containing bacteria after 24 hours of exposure, lightly rinsed with deionized water, dehydrated by serial dilution with EtOH and subsequently fixed using formalin solution. As evident from the photomicrographs, the ionic/chemical challenges exhibited no detectable surface irregularities at magnification up to 7000×. These assays further demonstrated that, although bacteria do adhere to the composite surfaces, they are not prevlalent on the modified silicone surfaces, and exposures to both the bacteria and ionic and chemical degradative factors present in the experimental growth media do not appreciably alter the smooth, defect-free surface characteristics of the activated polymer composites.

In more detailed embodiments, the surfaces of activated polymer composites of the invention remain essentially free of surface irregularities and defects that could promote microbial colonization, under a range of storage and use conditions, for extended storage and use periods. Under various environmental challenges (photodegradative, thermal, chemical degradative, microbial degradative), the surfaces of activated polymer composites remain free of cracks, pits, voids or other defects of sizes that could receive and shelter any microbial cell or colony. Expressed more distinctly, the activated composites of the invention posess smooth surfaces essentially free of pits, voids or cracks larger than any bacterial, yeast or protozoan cell. In certain embodiments, the surfaces of activated polymer composites of the invention remain free of structural defects including voids, pits or cracks having a largest void (i.e., wall to wall, or floor to opening) dimension of 1-5 μm or less, often no larger than 500 nm, 400 nm, 200 nm or even smaller. Activated polymer composite surfaces thus defined will have no more than 1-5 of these types of defects per square cm of surface area, and thus satisfy the definition of these polymer composites as having “microbially resistant” surface integrity (smooth, defect-free micro-texture).

Of additional surprising advantage, the activated polymer composites of the invention retain their novel “microbial surface resistance” marked by a smooth, defect-free surface architecture even after extended periods of use and exposure to environmental degradative influences. This is shown here following prolonged exposure to combined ionic, chemical and microbial degradative effects. In one important aspect, the polymer composites retain their microbial resistant surface character even after prolonged exposure to ionizing solutions (e.g., microbial growth media). Such solutions cause ion-exchange that leaches or dissociates some of the biologically active counter-ions from the polymer composite surface. More specifically, counter-ions present in ionic solutions ucouple ionic salt associations of the biologically active counter-ions with ion-exchange groups on the functionalized ion-exchange polymer salt (incorporated in fine particulate form in the polymer composite). This replaces some of the active counter-ions by salt exchange with new substitute counter-ions present in the ionic solution (e.g., Na+). This ionic degradative process is in fact a mechanism for “controlled activation and drug release” desired for some applications of the activated polymer composites. In these uses, the composites not only function by way of surface active chemistry, but in contact with physiological fluids and tissue and other ionic media they are able to dissociate some of the biologically active ionic agent in soluble form to exert biological activity away from the polymer surface (e.g., in a wound environment, or target tissue or compartment proximal to the polymer surface and contactable by solubilized biolgocially active ionic agent).

Of significance, “ionic degradation” influences (exemplified by prolonged exposure to physiological or other ionic solutions for prolonged periods of 6-24 hours or more, one to several days or weeks, even 1-6 months or longer) (unexpectedly) do not substantially alter the microbially resistant surface texture of the activated polymer composites. Despite the observed (and in many embodiments desired) mechanism and operation of “controlled activation and drug release” (discharging biologically active counter-ion from the exposed composite surface), the polymer composites do not lose their smooth surface architecture. They remain free of defects so as to retain “microbially surface resistance” (i.e., remain substantially free of defects large enough to provide anchorage or shelter for any microorganism or microorganism colony), despite this ionic degradation or discharge. In part, this is mediated by replacement of discharged, biologically active ionic agent on the polymer composite surface by counter-ions in the offending ionic medium, solution or tissue. Typically this counter exchange leaves no detectable surface defects, due to the generally small size of original, biologically active counter-ions loaded within the polymer composite (which will generally be replaced by similar small physiological ions, such as Na+). In some embodiments, surface maintenance and restoration will involve “recharging” the polymer composite surface using a salt solution comprising the original biologically activated counter-ion to replace discharged countereins (either by salt exchange to replace substituted counter-ions, or by re-association of the biologically active counter-ion with a functional group on the ion-exchange polymer left vacant after counter-ion discharge).

Notably, the studies here show that, despite prolonged exposure to ionic degradative factors, the biologically activated polymer composites of the invention do not shed or dislodge fine particulate ion-exchange polymer salt particles (embedded in the composite or composite surface), despite the observed discharge of biologically active ionic agent from association with the polymer salt particles over extended periods of time. Predictably, discharge by dissociation of a substantial portion of biologically active counter-ions from salt association with the fine polymer salt particles could diminishe their size and structural integration within the polymer composite, allowing them to be shed, dislodged or otherwise disintegrated from the surface of the composite. Natural ionic replacement (and artificial “recharging” as described above) of the polymer salts by ion-exchange in physiological and other ionic solutions surprisingly overcomes this problem. Unexpectedly, there is substantially no detectable loss of intact fine particulate ion-exchange polymer salt particles observed from polymer composite surfaces of the invention following prolonged exposure to ionic degradative factors as described. The surfaces of the polymer composites remain substantially free of defects (no greater than one defect per square centimeter of surface) of approximately equal or greater size than any of the fine particulate polymer salt materials employed (e.g., 200-500 nm, 500 nm-800 nm, 1-2 μm, 5-10 μm). This is also the case observed following prolonged storage and use of the subject polymer composites even under extreme conditions of thermal degradation (e.g., at temperatures above 200 degrees, 300 degrees, even 400 degrees for periods from one to several hours), photodegradation and chemical degradation.

Within the instant example, the milling vessel was topped off with non-solvent, sealed then placed (clamped) into a PM 100CM planetary ball mill. The sample milled for approximately 2 hours at 500 rpm. After this milling was stopped (more generally, when a desired milled particle size and uniformity are obtained), the fine particulate ion-exchange polymer salt is separated from the non-solvent (e.g., by evaporation) and media (e.g., by sieving).

### Example 57

### Preparation of Epoxy Incorporating IRP69:Ag

EPO-TEK 301 [Epoxy Technologies] was formulated to prepare a total 9.5 grams of epoxy for cure (4.00 grams of A and 1.00 grams of B. 0.57 grams of IRP69-Ag (SULFONATED POLYSTYRENE-CO-DIVINYLBENZENE Ag) in a Speedmixer cup. The mixture was mixed to evenly disperse the composite blend and the mixture cured by heating to 80° C. The antimicrobial properties of the surface were evaluated using an ASTM E2180 method to demonstrate a reduction in bacterial counts in excess of 4 logs.

### Example 58

### Preparation of Acrylic Incorporating IRP69:Ag (2.0 wt %)

Acrylic (SCIGRIP 40), a two-part compound was combined with IRP69-Ag (2.0 wt %). Handle with care and avoid the two components to come into contact with each other during the process. For this particular trial, IR69F-Ag was the additive utilized. IR69F is a crosslinked polymer of polystyrene sulfonate (PSS-DVB), with silver ion-exchanged onto it. The resin used in this particular example has been milled to approximately 400 nm. The acrylic material was evaluated against P. aeruginosa, E. coli, and S. aureus using an optimized (modified) ASTM E2180 assay (ASTM International, West Conshohocken, Pa., 2007) and the results demonstrated significant knockdown of the aforementioned pathogens.

### Example 59

### Preparation of Polyurethane (Tecoflex) IRP69-Ag Composite Material

Tecoflex EG-80A (9.8 grams) was dissolved into either THF or methylene chloride to about 25% solids and 0.20 grams of IRP69-Ag added and the mixture homogenized with stirring by hand. The solution was dispensed onto a glass plate and the solvent allowed to evaporate in a hood. The film was transferred to an oven set at 65° C. to completely remove residual solvent from the sample. The resulting material was a cosmetically acceptable tan color, maintained the characteristics of the parent polyurethane, and demonstrated antimicrobial effectiveness versus S. aureus, E. coli, and P. aeruginosa as determined from Kirby-Bauer disk diffusion assays. Small zones of inhibition were observed.

### Example 60

### Antimicrobial Self-Decontamination Surface Activity and Prevention of Contaminant Transfer Risk Potential by IRP69-Ag-Silicone (2.0 wt %)

The instant example demonstrates novel “self-decontaminating” surface activity of activated polymer composites of the invention. Additionally and by virtue of this novel surface active property, the activated polymer composite biomaterials provided herein secondarily function by reducing contaminant transfer risk in hospital, industrial and other environments. In exemplary hospital settings, traditional fomite surfaces made or coated with antimicrobially activated polymer composites of the invention are “self-decontaminating”, in that the original polymer composite surface (or regenerated or recharged composite surface) effects potent antimicrobial (e.g., bactericidal and bacteriostatic) activity, by both killing contaminating microbes in prolonged contact (sufficient for surface activity expression) with the composite surface, and also through microbistasis (without destroying or killing the microbe, rendering it functionally static as marked by an inability to colonize another surface or subject and survive or proliferate new microbes). In this exemplary study, the ability of E. coli to adhere to and persist on an exemplary extruded silicone rod (0.008 in. OD, Helix Medical Inc., Carpinteria Calif.) containing 2.0 wt % IRP69-Ag was tested for purposes of determining both contamination “resistance” of the composite surface, and its self-decontaminating activity. This assay in certain constructions also provides “time of kill” values for determining bactericidal activity (by providing values for how long test bacteria can remain adhered to the composite surface before dying).

From silicone slabs fabricated to include 2.0 wt % antimicrobially activated fine particulate ion-exchange polymer salts, disks were cut using a punch (hole) die, 6.0 mm). The silicone “punch-outs” (disks) comprising the activated fine particulate ion-exchange polymer salts are exposed to 10⁸ cfu/mL of bacteria to determine ability of the bacteria to adhere to the surfaces over time (as compared to non-activated silicone controls without antimicrobially activated fine particulate ion-exchange polymer salt added).

### Synthetic Urine Preparation

Dissolve following contents in DI Water

1. Calcium chloride (0.49 g/L)

2. Magnesium chloride hexahydrate (0.65 g/L)

3. Sodium chloride (4.60 g/l.)

4. Sodium sulfate (2.30 g/L)

5. Trisodium citrate dihydrate (0.65 g/L)

6. Disodium oxalate (0.02 g/L)

7. Potassium dihydrogen phosphate (2.80 g/L)

8. Potassium chloride (1.60 g/L)

9. Ammonium chloride (1.0 g/L)

10. Urea (25.0 g/L)

11. Gelatin (5.0 g/L).
pH of the medium should be approximately 6.1 after everything is dissolved. Adjustments to the pH can be made if necessary. Proceed to filter-sterilize solution by running it through a sterile 0.2 um filter into a sterile media bottle.
Once solution is sterile, proceed to add sterile TSB to yield a final TSB concentration of 10 g/L (stock concentration is 30 g/L)
Once TSB is mixed, this is working inoculum

### Inoculum Preparation

Start E. coli culture by inoculating 5 mL of TSB with single colony from streak plate. Allow culture to grow to confluence by incubating at 37° C. overnight, shaking at 220 rpm.
Perform second culture pass by adding 10 uL of confluent inoculum to 5 mL of freshly prepared synthetic urine. Allow culture to grow at 37° C. O/N, shaking at 220 rpm.
Once second culture reaches confluence, proceed to perform a dilution in synthetic urine to yield and OD600 absorbance of 0.2. This should be equivalent to about 10⁸ cfu/mL of bacteria, which is the desired bacterial concentration. Enough inoculums must be prepare to accommodate for triplicate of specimen(s) and control for 5 time points (in this case 30 inoculum tubes)
   Seeding of Bacteria onto Surfaces
Prepare silicone samples by punching out 6 mm punch outs from IR69F-Ag and blank silicone slabs (prepared by helix medical). Enough samples should be punched out to have triplicates for 5 time points.
Sterilize samples by placing in glass vials and autoclaving for 15 min.
Once samples are sterilized, seed bacteria unto samples by adding samples into (separate) tubes containing synthetic urine bacterial inoculum (see above)
Allow bacteria to adhere to surfaces by incubating inoculum samples with silicone specimens in a 37° C. incubator for 3 h, shaking at 220 rpm.

### Determining Self-Decontamination and Contaminant Transfer Risk Reduction

After 3 h of incubation, remove samples from incubator. Shake off excess inoculum and transfer samples to 2 mL of fresh, sterile urine (NO TSB). Continue to incubate samples that don't require work up by placing in 37° C. incubator (NO SHAKING). For example, if working up samples for T=3 h, the 8, 16, 24, and 32 h samples will be placed back in incubator.
To work-up samples, vortex tubes vigorously (30 s) in order to release adhered bacteria.
Perform serial dilutions of samples in nutrient broth or PBS. For blank (control) samples, a 1:100 and a 1:1,000 should yield single colonies. For test samples, a 1:100 should suffice, neats should also be plated.
Plate 200 uL of diluted/neat samples onto plate count agar plates using spread plate method. Incubate samples at 37° C. for 12-14 h or until single colonies appear.
Once colonies begin to appear, remove samples from incubator and take pictures. Count colonies and document results for data analysis
   The data from the aforementioned exposure experiment are revealed in FIG. 1. These data clearly demonstrate that the IRP69-Ag-modified silicone sample can reduce the amount of bacteria adhered to a silicone surface by as much as 6-logs within a period of 32 hours following exposure.

### Example 61

### Discoloration Reversal Process for Novel Silicone Polymers Containing Oligodynamic Metal as Biologically Active Ionic Agent

Exemplary activated polymer composites of the invention incorporate an oligodynamic metal such as silver as the biologically active ionic agent integrated within the composite through salt association with fine particulate ion-exchange polymer resins admixed within the polymer composites. These activated silicone rubber composites can be readily extruded (e.g., they have excellent green strength) to yield uniform tubing or other biomaterials, sheets, films and components. Upon standard curing of these and other, related biomaterials, it was observed that the silicone/metal composites develop darkened, reddish coloration characteristics. These cure-darkened color features are undesirable for many consumer, industrial and clinical uses. In particular they are simply less aesthetically pleasing in consumer contexts, and more so in clinical and industrial applications. As many of the uses contemplated for these materials relate to hygiene, where light coloration of materials is much preferred. Light coloration further enhances ability to detect soiling, surface defects, and contaminants (e.g., body fluids, caustic or toxic contaminants, etc.) The instant example details an important discovery for providing novel products and methods featuring lightened or non-discolored, biologically activated silicone/metal composites (actually lightened by reversal of cure-mediated discoloration. The silicone polymer composite containing silver (2.0 wt. % IRP69-Ag in Dow Corning Q7-4750) as fabricated by Helix Medical for Novion Technologies/Vachon) was cured for a standard curing period of 5-10 minutes at 150° C. This standard curing process yielded a darkly discolored, conventionally-cured silicone composite material. Surprisingly this pronounced discoloration was discovered to be reversible following alternate methods of extended or elevated temperature curing. In this example, the silver-activated silicone composite material was post-cured for an extended period of 1-2 hours at 150° C., during which extended curing the silicone-silver composite material lightened to a much more desired manila color. This novel color determining curing process, and the attendant results, can be further demonstrated using standard colorimetric methods.

### Example 62

### Activation of Chargeable Polymer Composite Surfaces by Post-Fabrication Surface Treatment

IRP69 (acid form, —SO₃H) was placed into DI water and stirred. 4.6 mEq/gram of Fe(OAc)₂ was added to the mixture (note: Fe(II) necessitates the use of ½ the molar amount given the divalent nature of iron (Fe(II)). The reaction was allowed to stir for 1-2 hours at room temperature and the presence of acetic acid (HOAc) was noted. The resulting resin (IRP69-Fe) is filtered, washed and dried. Milled to 1-10 μm and incorporated into silicone at 1-5 wt %. The silicone is exposed to hydrogen peroxide 3% (non-stabilized) in the presence of methylene blue and the observation of a decolorizing from blue to gray is indicative of the formation of superoxide.

This novel “Fenton Chemistry” charging reaction to yield potently antimicrobial superoxides at the surfaces of polymer composites (through manual post-fabrication activation, involving “surface activation” or “surface charging” by exposure of the composite surface to a peroxide or other charging chemical or solution (to generate a new, biologically active chemical by-product at the activated or charged surface of the polymer composite), provides yet another conceptual breakthrough in the fields of biomaterials production and application. Comparable results were obtained with another polymer composite of the invention incorporating an iron additive.

As exemplified by the Fenton Chemistry model for surface activation, surface charging of the foregoing exemplary polymer composites occurs when a divalent metal ion, typically iron, is exposed to peroxide, leading to the formation of a radical species (e.g., superoxide (O₂⁻). Superoxides have strong antimicrobial properties, and thus their renewable production by surface activation here evinces one embodiment of a surface activate-able or surface re-chargeable polymer composite. This activation potential is renewable in the sense that the activation can be repeated for the same polymer composite surface, to yield multiple rounds of activation (e.g., successive events of superoxide production at the polymer composite surface, manually controlled by simply spraying or wiping the surface with an activating solution such as hydrogen peroxide). These exemplary results for superoxide generation are illustrated below, while it will be appreciated that many distinct “activating” or “charging” materials and methods are contemplated within the scope of teachings herein. We have demonstrated that IRP69-Fe(II), in 3 mL of methylene blue solution (0.1 mmolar) with and without the addition of unstabilized peroxide reveals no color change. When 300 μL of 30% non-stabilized peroxide solution (to yield a final peroxide concentration of 3%) were added to the corresponding tube (+H₂O₂) and the sample was allowed to react, slight ecolorization of the solution occurred instantaneously, with full decolorization (bleaching) of the solution complete after 2 min. (C) Silicone test swatches containing the iron additive (2.0 wt %) were also evaluated by adding methylene blue solution (0.1 mmolar) on to the surface and then adding peroxide (300 μL of 30% unstabilized peroxide). After 15 min, decolorizing of the solution (bleaching) above the test was observed in the presence of peroxide. It is important to note that this bleaching effect was not observed when peroxide was added to resin in the acid form (no Fe(II)). This experiment reveals that the IRP69-Fe(II) derivative (SC-GARDION-Fe) can be used to generate pathogen killing superoxide with the treatment of a surface with uninhibited hydrogen peroxide. This unanticipated result can be applied to the fabrication of connectors for use in central venous and other catheters where decontamination is an important element of preventing blood and other infections.

### Example 63

### Preparation and Testing of Hydrophilic Polyurethane Foam Incorporating 5 wt % IRP69-Ag

A 10 gram sample of IRP69-Ag (1-10 micron particle size) was provided to Rynel Inc.(Wicasset, Me.) and approximately 100 grams of hydrophilic open cell hydrophilic polyurethane foam (SE-3) was provided for testing. Kirby-Bauer disk diffusion assays using a 6 mm punched disk against Pseudomonas aeruginosa (PA). Enterococcus faecalis (EF), and Staphylococcus aureus (SA). The samples demonstrated zones of 0.08 mm vs. PA, 0.08 mm vs. SA, and 0.08 mm vs. EF were recorded. Control foams (polyurethane only) showed no zones under the same conditions.

### Example 64

### Preparation and Testing of Hydrophilic Polyurethane Foam Incorporating 5 wt % IRP69-Benzalkonium

A 10 gram sample of IRP69-benzalkonium (1-10 micron particle size) was provided to Rynel Inc.(Wicasset, Me.) and approximately 100 grams of hydrophilic open cell hydrophilic polyurethane foam (A4) was provided for testing. Kirby-Baucr disk diffusion assays using a 6 mm punched disk against Pseudomonas aeruginosa (PA), Enterococcus faecalis (EF), and Staphylococcus aureus (SA). The samples demonstrated zones of 0.00 mm vs. PA, 0.17 mm vs. SA, and 0.08 mm vs. EF were recorded. Control foams (polyurethane only) showed no zones under the same conditions.

### Example 65

### Preparation of Polypropylene (PP) Incorporating 2.0 wt % IRP69-Ag

75 grams of IRP69-Ag (500 nm average particle size) was provided to LTL Color Compounders, Inc. (Morrisville, Pa.) and the material compounded into medical grade polypropylene. The compounding effort yielded 4 lbs of modified PP and 20 molded coupons of a light tan coloration. Examination of the surface revealed excellent characteristics and antimicrobial testing by ASTM E2180 demonstrated excellent effectiveness against Pseudomona aeruginosa.

In addition to resolving fabrication constraints, improved stability of attached cations was demonstrated by thermal gravimetric analysis. For example, benzalkonium chloride (melting point=35° C.) is an inhibitor of cure in 2-part platinum curing silicones. In the event that the simple salt did not inhibit cure, it would be molten during cure and thus leak or ooze from any curing (molded or extruded) material. However, in the disclosed polymer salt association with ion-exchange resin, concerns over melting are eliminated allowing unimpaired crosslinking of polymers in the subject polymer composites. Further surprising, curing is not inhibited when activated polymer salt particles are incorporated into 2-part platinum cured silicones.

Thermal stability of a benzalkonium salt of IRP69 (IRP69-benzalkonium) was measured and compared to thermal stability of benzalkonium chloride (simple salt). The thermogravimetric profile demonstrated that benzalkonium chloride begins to decompose at about 200° C. whereas the IRP69-benzalkonium active biocide remains very stable to at least 300° C. (close to the optimal processing temperature polyethylene terephthalate), unexpectedly providing for stable incorporation of the subject biologically active materials within composites useful to create fibers, threads, woven textiles and fabrics incorporating these thermally demanding materials.

### Example 65

Preparation of Ligand Stabilized Multivalent Metal Cationic Salts IRP64-Cu, a weak cation (WC) Cu Resin, or IRP69-Cu, a strong cation (SC) Cu Resin (or other multivalent metal, for example zinc) was weighed in a centrifuge tube. Deionized water was added to wet the resin followed by an excess of concentrated Ammonium Hydroxide, the resin should change to dark blue. The solution was mixed with a vortexer followed by centrifugation. The aqueous solution was decanted yielding the Cu(NH₃)₂ SC or WC complex. The complex has also been prepared by flowing ammonia gas through column containing the solid SC or WC copper exchange resin.

### Example 66

### Polymer Composite Paints Comprising Stabilized Strong and Weak Cation-Exchange Copper (IRP64-Cu and IRP69-Cu, SC-Cu and WC-Cu) Derivatives

Glidden Interior Paint+Primer GLN6441 (Glidden, Cranberry Township, Pa.) was coated on a glass surface and placed in a 60 C oven for 12 hours to evaporate all liquid content producing a solid coat of paint. The difference in mass was calculated to determine the percent solids (51.6% w/w). 0.4128 g of IRP69 or IRP64 ammonia stabilized copper powder (1-10 um) was weighed in a speed mixer cup and admixed with 39.6 g of GLN6441 (2% w/w solids) to form a biologically activated polymer composite paint. The resulting liquid composite mixture was blended using a speed mixer to provide a paintable copper polymer composite, without adversely changing the viscous properties of the paint.

Copper samples were prepared as described above using a GLN6441 polymer precursor base. Samples of IRP69-Cu(NH₃)ₙ and IRP64-Cu(NH₃)ₙ were made at 0.75% and 1.5% solids. A sample of IRP69-BA was made at 2% solids. The samples were painted twice onto grade GF/D Whatman filter paper (General Electric Healthcare, Little Chalfont, Buckinghamshire HP7 9NA) and allowed to dry for 24 h in a 60 C oven. The samples were extracted in 0.9% NaCl for 3 days. The samples were sterilized, cut into 1×1 inch samples and tested against a variety of organisms in the ASTM 2180. Results are shown in Table 11.

Table 11 demonstrates the results of a modified ASTM E2180 assay (ASTM International, West Conshohocken, Pa.) using an inoculum of 10⁶ of various pathogenic bacteria against composited GLN6441 Interior Paint+Primer (Glidden) (incorporating sulfonated polystyrene-co-divinylbenzene Cu (2.0% wt/wt) compared to an non-modified control GLN6441 Interior Paint+Primer. The data reveal that incorporation of Cu-modified, antimicrobial and antifouling polymer salt particles of the invention reduced bacterial activity against subject test coatings by almost 6-logs, with no significant adverse impacts on viscosity, aggregation/clumping, paintability, drying characteristics, hardness, durability or other observed performance characteristics of the paint.

These results demonstrate a surprisingly potent and long-lasting efficacy of GARDION BIOCIDE™-modified acrylic latex enamel paint. These and other polymer-based paints and coatings are exceptionally useful tools in sterial management of clinical, institutional, food processing and other environments, to minimize pathogenic colonization, residence and growth on fomites, among many other related antimicrogial, antifouling and anti-biologic uses as described herein.

TABLE 12
Table 12 - ASTM E2180 log reduction results observed for 10⁵ CFU
exposures to Glidden paint modified to include GARDION-Cu BIOCIDES
(STRONG AND WEAK CATION VERSIONS, IRP69 and IRP64 respectively).

| | Log Reduction for Each Organism exposed to paint surfaces | | | | | |
|---|---|---|---|---|---|---|
| Sample | Escherichia | Staphylococcus | Enterococcus | Staphylococcus | Klebsiella | Pseudomonas |
| ID | coli | Epidermidis | faecalis | aureus | pneumoniae | aeruginosa |
| Control | 0 | 0.19 | −0.63 | 0.53 | 0 | 0 |
| (T + 24) | | | | | | |
| IRP69- | 5.74 | 5.74 | 2.85 | 5.74 | 5.74 | 3.51 |
| Cu 0.75% | | | | | | |
| IRP69- | 5.74 | 5.74 | 5.74 | 5.74 | 5.74 | 5.74 |
| Cu 1.5% | | | | | | |
| IRP64- | 5.74 | 4.26 | 2.28 | 5.74 | 5.74 | 4.05 |
| Cu 0.75% | | | | | | |
| IRP64- | 5.74 | 5.74 | 5.74 | 5.74 | 5.74 | 5.74 |
| Cu 1.5% | | | | | | |
| IR69- | 0 | 2.91 | 2.38 | 5.74 | 0 | 0 |
| BA-2% | | | | | | |

### Example 67

### Polymer Composite Antifouling Paints Comprising Amonia Stabilized Multivalent Biocidal Metals Associated With Strong Cation or Weak Cation-Exchange Resins

Aluma Hawk AH7000 aluminum boat paint (Sea Hawk, Clearwater, Fla. 33762) was coated on a glass surface and placed in a 60° C. oven for 12 hours to evaporate all liquid content, producing a solid coat of paint. The difference in mass was calculated to determine the percent solids (70% w/w). 0.5600 g of IRP69 or IRP64 ammonia stabilized copper particulate (activated polymer salt-1-10 μm particle diameter) was weighed in a speed mixer cup and admixed with 39.6 g of AH7000 as prepolymer base (comprising 2% w/w solids). The resulting mixture was blended using a speed mixer to produce a copper-activated polymer composite paint solution, without changing viscosity or other performance properties

### Example 68

### Marine Polymer Composite Antifouling Paint

Another example of a useful antifouling paint/coating of the invention employed a marine antifouling paint, selected as Sea Hawk Aluma Hawk paint (70% solids by wt) (comprising polymer precursors for formulation of a polymer composite mixture of the invention, as described). To this polymer base was added SC-GARDION™-Cu—NH₃ (ligand-stabilized Cu(II) biocide), 3.0% w/w. A 10×10 inch aluminum sheet was painted with this marine antifouling polymer composite mixture to a uniform thickness. This dry test article was placed into a secure test site within the Pacific Ocean and retrieved after 16 weeks. The surface was nearly pristine as marked by the absence of visible growth or encrustation by marine algae, films, or macroorganisms, including crustaceans, none of which visibly resided on the test surface upon close inspection after the coated article was lightly shaken and removed from the sea water. Coated surfaces lacking biocide and uncoated surfaces are heavily fouled under these conditions following the same exposure period. Additional testing will further detail that antifouling paints and coatings of the invention mediate substantially greater inhibition of marine fouling of all kinds in side-by-side comparison to other commercial antifouling paints and coatings containing more toxic biocidal agents that show greater leaching of toxic agents into surrounding marine ecosystems than the coatings and paints of the invention.

### Example 69

### Marine Polymer Composite Antifouling Paints and Coatings Incorporating Monovalent Copper Integrated in a Strong Cation Exchange Resin (Cu(I)-SCE)

The strong cation-exchange resin IR69F-Na (Dow Chemical Company, Midland, Mich.) was stirred in an excess amount of deoxygenated, deionized water with the aid of a mechanical stirrer in the absence of light. To the stirring mixture, 4.5 mEq/gram of Copper (I) Chloride was added and the mixture stirred until the CuCl was taken up by the resin. ˜1 hour. The resulting solid was rinsed with deionized water until no measureable copper was present in the filtrate (as determined, e.g., by MQuant copper test strips (EMD Millipore, Billerica, Mass.)). The addition of copper totaled about 40% of theoretical incorporation maximum (1.85 mEQ/gram) and the solid material had a yellow-orange color and not the green color generally observed for Cu(II) derivatives of the invention described here. The solid was transferred to a vacuum oven at 70° C. for 24 h or until dry to yield a light and heat stable modified Cu(l) salt. The resulting, copper-activated polymer salt material was milled to a fine particulate (average 1-10 micron) particle size, as determined by light scattering.

Release of Cu(I) was verified by adding deionized water to the clean solid, measuring the aqueous layer for Cu using a test strip and showing that no copper was present (i.e., it remained stably associated within the polymer salt complex). Subsequently brine was added to the solution and the aqueous layer was remeasured for Cu shortly after the brine addition. After the addition of brine to the Cu(I)-SCE the concentration of copper in the aqueous layer was beyond the detection limit of the test strip (300 ppm). The copper-activated polymer salt particulate material was transferred to a vacuum oven at 70° C. for 24 h until dry, producing a light and heat stable modified Cu(I) polymer salt. This material can be routinely milled to 1-10 micron particle size as measured by light scattering.

The foregoing copper-activated fine particulate polymer salt material was subsequently incorporated into a silicone material (MED-4950) and tested against Staphylococcus aureus using the ASTM E2180 assay. The composite material demonstrated a 5.2 log reduction against Staphylococcus aureus.

Cu(I) is routinely used as an antifouling component of coatings for ocean-going vessels. Generally, this use is in the form of Cu(I)O, with very high concentrations of oxide are used (>30 wt %) to ensure antifouling activity. The novel polymer composites of the invention allow for binding and steady-state kinetic release of both Cu(I) and Cu(II) species, in a meterable fashion (adjustable by selectable resin loading, polymer cross-linking, type of biocide, and other means and materials described herein), to achieve both performance and environmental improvements over current technological approaches.

### Example 70

### Stability of Composite Paints and Coatings to Ionic Solutions

A glass filter paper with Glidden latex enamel infused with the IRP69-Cu additive (1.5 wt %) was exposed 0.9% NaCl solution overnight at 37° C. The filter paper was subsequently thoroughly rinsed with DI water to ensure that no excess copper acetate salt was on the surface. The Glidden acrylic latex enamel paint was extracted for five days and the painted surface was exposed to bacteria using the ASTM E2180 assay (data not shown) using regular paint as a negative control and IRP69-Cu prepared by standard methods as the positive control. The “extracted” surface showed antimicrobial efficacy demonstrating the ability of the resin to resist extraction through a paint matrix.

### Example 71

### Antifouling Activity of Composite Paints and Coatings

Paint ASTM testing method ASTM D2574-86 (ASTM D2574-86 (Test Method for resistance of Emulsion Paints in the Container to Attack by Microorganisms) represents one of many available, well known and widely used testing method for determining resistance of polymer emulsion paints against “fouling,” including to prevent or reduce colonization and/or growth of microorganisms (here antifouling applies to both liquid polymer composite paints during manufacture and storage, and to solid (i.e., cured-including viscous gel, semi-solid, and flexible solid cured paints) polymer composite paints after they are applied as a coating or laminate and cured/dried. The term “antifouling” as applied to paints, coatings, materials and articles of manufacture of the invention will be understood to encompass antimicrobial and antifungal activities of biologically activated polymer composite paints of the invention, as well as all other anti-biologic activities (i.e., direct or indirectly impeding biological activity that affects colonization, growth, reproduction and/or survival of one or more organism(s) targeted for control)—such as anti-algal (i.e., inhibiting micro and/or macroalgae), antifungal (i.e., inhibiting fungal spores and other life history stages of molds, mildews, and macrofungi), anti-zootic (i.e., inhibiting any of a range of animal organisms targeted for control—for example in marine environments, crustaceans (including barnacles), enidarians (e.g., anenomes and corals), encrusting and boring worms, encrusting and boring mollusks, and others).

To illustrate antifouling activities of the paints and coatings of the invention, IRP69-Cu additive infused polymer composite paint prepared as above, and the still-liquid (i.e., not yet cured) polymer composite mixture was exposed to representative test microorganisms three times at T0, and after one week, and two weeks. At each time interval liquid paint samples were taken, diluted and plated for evaluation of the ability of the paint to resist microbial contamination. The polymer composite paints demonstrated potent resistance to microorganism contamination compared to relevant control samples. Additional studies using ASTM E2180 and JISZ2801 test protocols for paint are underway and will further exemplify the potent anti-biologic and antifouling activities of paintes and coatings provided herein.

### Example 72

### Antifungal Composite Paints and Coatings

ASTM G21 (Standard Practice for Determining Resistance of Synthetic Polymeric Materials to Fungi) was used to evaluate the IRF69-CuNH₃ composited in a polymer paint to demonstrate potent antifungal activity. This test determines the effect of fungi on certain properties and characteristics of synthetic polymeric materials that may include, but are not limited to paint, plastics, paper, cardboard, and drywall (all of which materials can be effectively anti-biologically activated by incorporation of fine particulate activated polymer salts made according to the invention). In this test method as applied to paints and coatings of the invention, a high concentration suspension of spores of interest was prepared. The resulting nutrient agar salt slurry containing the spores was poured into sterile petri dishes. Antifungal composite paint was evenly coated and dried onto test tabs as test specimens, and these were placed on the solidified agar, incubated for 28 days under 90% humidity, and then evaluated for growth. Comparably handled and processed control specimens were produced using the same paint base not composited with the antifungal activated fine particulate polymer salt IRF69-CuNH₃. These experiments revealed a greater than four log reduction in fungal colonies within the inoculated agar between test and control samples. In a majority of test cases no fungal growth whatsoever was observed following the incubation.

### Example 73

### UV-Acrylate Paints and Coatings for Metal and Concrete

Unicryl (UV-curing acrylic) resin was combined with 2.0 wt % SC-GARDION-Cu and the composition was applied to a primed metal panel and allowed to sit at room temperature in a hood for 2 hours, and the panel was irradiated 20 seconds (4 passes at 5 seconds per pass) with a D-bulb (20.5 Joules/cm2) in a Fusion UV curing station. All coatings were tack- and print-free after irradiation. According to these results, the activated polymer composite paints and coatings of the invention can be utilized to coat other metals, wood, concrete, fiberglass, carbon fiber materials, and a broad range of other solid materials. Additionally, these results evince utility of a range of polymer-based paints and coatings for useful integration and application of anti-biologic polymer paints and coatings, e.g., employing urethane acrylates, acrylic acrylates, and epoxy acrylates, for a diverse range of uses (e.g., for coating surfaces/fomites in food processing plants, hospitals, public transportation, manufacturing facilities etc.) and a diverse range of receiving surfaces (e.g., construction materials, HVAC ducts and surfaces, equipment, furnishings and fixtures, etc.), wherever biocidal activity may be important. Exemplary UV-coatings containing a GARDION™ biocide can be applied for example using a field-applied coating system, such as a CYTEC system, or equivalent. ASTM E2180 studies revealed, among other observed activities, that a representative range of different representative antimicrobial polymer composite paints and coatings of the invention exert potent biocidal and surface-to-surface transfer inhibition activities against inoculated Staphylococcus aureus when coated and dried onto a variety of fomite surfaces.

### Example 74

### Surface Polishing Recharge of Painted and Coated Surfaces

Lightly polishing different representative anti-biologic (e.g., antimicrobial, antifungal, antifouling) polymer composite paints and coatings of the invention, after application and curing, removes a thin layer of the cured coating, providing a “recharged” or “regenerated” or “self-decontaminating” surface. These highly useful properties have been demonstrated using a range of anti-biologic assays and a range of anti-biologic paints and coatings. To illustrate, exemplary antimicrobial polymer composite paints and coatings were tested as described using the ASTM E2180 assay, demonstrating potent antimicrobial activity, further demonstrated to impart powerful inhibition of surface-to-surface contamination potential from a coated surface to an uncontaminated receiving surface. In general, paints and coatings of the invention, when applied and cured onto a fomite surface will mediate greater than a 25% reduction in transfer potential of pathogenic bacteria to an uncontaminated receiving surface (or tissue, wounds, or media such as plasma) following direct contact exposure of the receiving surface with a coated surface, device, textile or article). In certain embodiments, this potent reduction of pathogenic transfer potential will be greater than 50%, greater than 75%, up to 95%-100% reduction of transfer potential (i.e., surface-surface contamination risk) (e.g., compared to transfer potential observed for like-inoculated, incubated and treated controls coated with non-activated (non-composited base polymer) coatings). Comparable levels of anti-biologic activity is observed for a representative range of coatings following polishing recharge, against a diverse array of test organisms as described herein. In certain detailed embodiments. GARDION™ BIOCIDE paints and coatings may employ into solvent based systems (lacquers), which is particularly useful for coating concrete, wood, and certain metals. The crosslinked structures of these polymer systems prevent them from dissolving, although in some cases and certain solvents nominal swelling was observed.

### Example 75

### Preparation of Polypropylene-Ag-SCE Polymer Composite Textile

Polypropylene (PP) pellets (Exxon Mobil homopolymers resin) were placed into a glass beaker and placed into a 200° C. oven and after 45 minutes the polymer was melted. To the melted PP. GARDION™ SC-Ag was added (2 wt %) and the mixture blended with the aid ofa PTFE-coated spatula until homogeneous. The mix was poured onto a PTFE sheet and allowed to cool to a slab/film. The residual melt was used to draw some crude fibers. The material formed a golden-colored solid. ASTM E2180 evaluation against a 10⁵ inoculum of Staphylococcus aureus demonstrated a 6.4 Log reduction. The properties of the slab/film were good and the fiber retained good flexibility.

Applications of these and related textiles incorporating biologically activated polymer composites of the invention include, for example tooth brushes (including bristles), non-woven textiles (spun bond fibers), sutures, and the like). Similar materials including Nylon 6 (m.p. 220° C.) can be used by comparison as the SCE and WCE materials are stable at these temperatures.

### Example 76

### Q7-4750-SC-GARDION™-Ag Composition

In a sigma blade mixer 4.0 lbs. of Dow Corning (Part A) Q7-4750 (addition curing (Pt-curing)) silicone rubber was combined with 72 grams of Ag-SCE and the mixture blended until homogeneous to yield a 4.0 wt % loaded composition. The 4.0 wt % loaded part A was placed onto a 2-roll mill and combined with 4.0 lbs. of part B until the composition was completely homogeneous (Q7-4750/SC-GARDION™-Ag). Ag=silver ion.

### Example 77

### Q7-4750-SC-GARDION™-BA Composition

In a similar fashion, 4.0 lbs. of Dow Corning Q7-4750 part A was combined with BA-SCE (72 grams) and the mixture blended and subsequently combined with 4.0 lbs. of part B. BA=benzalkonium ion.

### Example 78

### Q7-4750-SC-GARDION™-BA/Ag Composition

In a similar fashion, 4.0 lbs. of Dow Corning Q7-4750 part A was combined with a binary mixture of Ag-SCE and BA-SCE (36 grams of each) and the mixture blended and subsequently combined with 4.0 parts of part B. BA=benzalkonium ion, Ag=silver ion.

### Example 79

### Preparation of SC-GARDION™-Ag Silicone Tubing

Q7-4750-SC-GARDION™-Ag composition was extruded into tubing and cured in a vertical curing tower to yield pristine golden-colored tubing (0.080 OD, ID0.056). The tubing passed visual inspection and is stable on the shelf in excess of one year. ASTM E2180 evaluations of the modified silicone tubing against a variety of microorganisms demonstrated that the modified silicone retained potent antimicrobial activity commensurate with the range and values described above.

### Example 80

### Preparation of SC-GARDION™-BA silicone tubing

Q7-4750-SC-GARDION™-BA composition was extruded into tubing and cured in a vertical curing tower to yield pristine tubing (0.080 OD, ID0.056). The tubing passed visual inspection and is stable on the shelf in excess of one year.

### Example 81

### Preparation of Co-Extruded Silicone Tubing (SC-GARDION™-Ag Outer/SC-GARDION™-BA/Ag Inner)

Unvulcanized Q7-4750-SC-GARDION™-BA/Ag and Q7-4750-SC-GARDION™-Ag Compositions were loaded into their respective hoppers in the extruder and the compositions co-extruded and cured to yield a tube with an outer layer of Q7-4750-SC-GARDION™-Ag and an inner layer of a mixed composition Q7-4750-SC-GARDION™-(1:1) BA/Ag (2.0 wt. %). The outer diameter of the tube was 0.080 in. (80 mil) and the wall thickness of each layer 10 mil (0.010) leaving an ID of 0.040.

### Example 82

### Preparation of Molded Silicone Composite Articles

Slabs of silicone elastomers were molded at 200° C. to yield an 8.0×8.0 inch×6.0 mm and ASTM (D412 die C) dog bones were cut. ASTM D 412 specifies a dumbbell shaped specimen. The specification describes 6 options for the sample dimensions, but the preferred sample is “Die C”. Die C has an overall length of 115 mm (4.5 inches) with a narrow section 33 mm (1.31 inches) long. This provides a gauge length (benchmark) 25 mm (1 inch) long and a gauge width of 6 mm (0.25 inch). Tensile testing was carried out and the results were all within the manufacturer's specification. Mechanical testing data is shown in the following Table 13.

TABLE 13
Table 13 - mechanical testing (tensile and durometer) data for
Q7-4750 modified to include GARDION ™ BIOCIDES

| Silicone Composite | Durometer | Tensile Strength | % Elongation |
|---|---|---|---|
| Control (Q7-4750) | 45-55 | 1200 psi (min) | 750% (min) |
| Ag-SCE, 2% | 50 | 1353.8 ± 28.5 | 980.8 ± 13.4 |
| Cu-SCE, 2% | 57 | 1140.4 ± 43.9 | 882.7 ± 27.0 |
| Cu/Ag-SCE (50:50), 2% | 50 | 1274.7 ± 40.5 | 918.1 ± 16.0 |
| BA-SCE, 2% | 56 | 1218.1 ± 38.6 | 884.7 ± 25.9 |

### Example 83

### Structures Prepared by Mandrel Dipping Liquid Latex Formulated to Include BA-SCE

Liquid latex body cosmetic (Maximum Impact) was placed into a Max 100 Speedmixer cup and 2.0 wt % BA-SCE added and the mixture blended at 3000 RPM for 2 minutes. The mixture was used for dipping a mandrel (test tube) and the mixture allowed to cure. After 24 hours the fingers were removed and portions cut to form disks. ASTM E2180 evaluation against a 10⁵ inoculum of Staphylococcus aureus demonstrated a 6.35 Log reduction.

### Example 84

### Structures Prepared by Mandrel Dipping Liquid Nitrile Elastomer Formulated to Include BA-SCE

Zetpol ZLX HNBR LATEX was placed into a Max100 Speedmixer cup and 2.0 wt % BA-SCE (1 micron particle size) added and the mixture blended at 3000 RPM for 2 minutes. The mixture was used for dipping a mandrel (test tube) and the mixture allowed to cure. After 24 hours the fingers were removed and portions cut to form disks. ASTM E2180 evaluation against a 10⁵ inoculum of Staphylococcus aureus demonstrated a 6.28 Log reduction.

### Example 85

### Foams Prepared as Diagnostics for Small Ion Recovery and Detection

Bayer hydrophilic foam FP503 was formulated to include 5 wt % Na-SCE (IRP69-sodium, 1 micron particle size). The finished foam was exposed to a solution of iron (II) chloride (20 PPM) for 48 hours and the foam removed, soaked in DI water for 8 hours and dried. A small portion of the foam was submitted for ICP analysis (acid digestion). The inductively coupled plasma atomic emission spectroscopy (ICPAES) analysis revealed a strong Iron signal indicating exchange onto the resin backbone. This proof-of-concept is a demonstration that a high surface area construct that is fabricated from a small particle size ion-exchange resin distributed in a polymer matrix can be used as a diagnostic tool that can be utilized for the evaluation of ground water, water sources, agribusiness land by placement of the test substrate into the location for some period of time and subsequently evaluating the test substrate for metals or organic analyses for the determination of pollutants or the presence of cations or anions that may be indicative of the presence of fertilizer for example (nitrates, iron, sulfates, lead, arsenic etc.). This assumes that the standard curves can be generated (which they can) and that manufacturing can be made reproducible, and the matrix does not contain any ions (such as those used for catalysis in making a foam) that can interfere with the measurements. It should be noted that a foam is not a requisite for making a device that can function in this capacity. For example a polypropylene (metallocene catalyzed to include Zr or Ni for example) can be fabricated as a coating onto a solid metal or polymer substrate that can be pushed into the ground. After some time the polypropylene can be removed and evaluated by ICPAES. It may be that the PP can be placed onto the substrate in the form of a film or a hollow (porous) rod may house a high surface area foam that can be removed from the rod once it is removed from the site to be analyzed. This same approach may be employed using an adsorbent resin such as Amberlite XAD1180N, embedded as a small particle form into a high surface area substrate (foam) in order to adsorb organic impurities that can subsequently be evaluated using a mass spectrometric method of determination.

### Example 86

### Foams Prepared for Vacuum Assisted Closure Applications

Bayer hydrophilic foam FP503 was formulated to include Ag-SCE, BA-SCE, Chlorhexidine-WCE, and Tetracycline-SCE at 2.0 wt %. Each of the foams were tested by Kirby-Bauer disk diffusion assays against Staphylococcus aureus and BA-SCE, tetracycline-SCE, and chlorhexidine-SCE demonstrated clear zones. Evaluation against Staphylococcus aureus using the ASTM E2180 assay revealed log reductions of at least 5.0 for each of the foams (with a 10⁵ inoculum).

### Example 87

### Food Packaging (Polypropylene)

The polypropylene Ag-SCE example serves as an optimal example for food packaging because polypropylene is the most common take out packaging material used today and foamed polypropylene is used other food containers. Polyurethane foam serves as an example for foamed polystyrene

### Example 89

### Sexual Prosthetics

A silicone dildo was dip coated using a MED 4950 silicone lacquer (heptane, 10-20 wt % solids) incorporating 3.0 (dry wt %) (IRP69-Cu) Cu-SCE and the lacquer allowed to dry. The silicone was cured at 180° C. for 10 minutes to yield a strongly adherent coating with superb frictional stability. The coating possessed a slight blue color reflecting the color of the resin additive. Evaluation of the coating against Staphylococcus aureus, Proteus mirabilis, and Candida albicans demonstrated log reductions in excess of 5.0 against each organism.

### Example 90

### Silicone Adhesives

Nusil Technology MED1050 RTV adhesive was mixed by hand to incorporate 2.0 wt % Ag-SCE. A portion was allowed to cure overnight and the solid silicone tested against Staphylococcus aureus using an ASTM E2180 assay. The cured adhesive demonstrated a 6.18 log reduction following a 10⁵ inoculation.

### Example 91

### Acrylic Adhesives for Wound Dressing Applications

“Arosct AGX L” (Ashland Inc.), was weighed into a tared speedmixer cup and 3.0 wt % of the benzalkonium biocide (SC-GARDION™-BA) was added and the mixture combined with the aid of a PTFE coated spatula. After initial mixing, the mixture was placed into the speedmixer with two 4 mm ceramic cylinders and the speedmixer was run at 2500 rpm for (2) 1 minute cycles. A visual inspection was performed to look for any particulates. In the event that the mixture was non-homogeneous, the mixture was speed-mixed for another 1 minute cycle. The acrylic was applied to the substrate of choice and allowed to dry. The same process was carried out using WC-GARDION™-CHX and SC-GARDION™-Ag without any issues. The dried acrylics were tested using ASTM E2180 and shown to be effective against multiple organisms. The BA composition was more effective against gram-positive organisms whereas Ag and CHX demonstrated broad antimicrobial activity with SC-GARDION™-Ag demonstrating activity against fungi (Candida albicans, Aspergillus fumigatus). As such the adhesive patch can be used in the treatment of ringworm for example.

### Example 92

### Silicone Gel Materials for Use in Prosthetic Devices

Prosthetic devices, particularly leg devices, require cushioning inside the device to prevent pressure damage to tissue at the stump surface. Silicone gel was formulated in a Speedmixer cup to include 2.0 wt % Ag-SCE. The silicone was poured onto a sheet and cured at 150° C. for 15 minutes. The solid gel was evaluated using an ASTM E2180 against Staphylococcus aureus. The material demonstrated a 5.88 log reduction in the organism. Evaluation of the gel against Candida albicans demonstrated an equally effective 5.9 log reduction.

### Example 93

### ASTM E2180 of Tecophilic TG-500 Coated Polyester Substrate

Solutions of IRP69-Ag (1-10 micron) and IRP64-Chlorhexidine (1-10 micron were made at 5% and 10% solids using a method described for Tecophilic polyurethane above. Polypropylene mesh coverslips were dip coated and set aside to dry for 24 hours before they were sterilized to challenge against multiple bacteria in the ASTM E2180 with MRSA and Escherichia Coli. Results are shown in tables 14A and 14B, these data fully demonstrate potent the antimicrobial efficacy, of TG-500 Hydrogel Dressings against MRSA using the modified ASTM E2180 method described.

TABLE 14A
Table 14A - ASTM E2180 (MRSA) for TG500
(polyurethane) coated polyester fabric

| Pathogen - MRSA | |
|---|---|
| Sample ID | Log Reduction |
| Ag-69 5% | 5.63 |
| Ag-69 10% | 5.63 |
| CHX-69 5% | 5.88 |
| CHX-69 1-% | 5.88 |

TABLE 14B
Table 14B - ASTM E2180 (E. coli) for TG500
(polyurethane) coated polyester fabric

| Pathogen Escherichia coli | |
|---|---|
| Sample ID | Log Reduction |
| 69-Ag5% | 6.66 |
| 69-Ag10% | 6.66 |
| 69-CHX5% | 6.66 |
| 69-CHX10% | 6.66 |
| 69-Cu5% | 6.66 |
| 69-Cu10% | 6.66 |

The above data demonstrate the log reduction results from a modified ASTM E2180 (ASTM International, West Conshohocken, Pa. 2007) assay using an inoculum of 10⁶ MRSA of E. coli against IRP69-Ag (silver-Sulfonated polystyrene-co-divinylbenzene)-modified TG-500 hydrogel dressings Ag (5 and 10 wt. %) and IRP69-CHX (chlorhexidine) (5 and 10 wt %) compared to an non-modified control hydrogel dressings. These data reveal that the IRP69-Ag-modified and IRP69-CHX-modified dressings were capable of killing essentially 100% of the inoculum.

### Antiviral Testing

ASTM E1053-11-Standard Test Method to Assess Virucidal Activity of Chemicals Intended for Disinfection of Inanimate, Nonporous Environmental Surfaces was used to evaluate the antiviral effectiveness of IRF69-Ag, exemplified in hydrophilic foams. A glass Petri dish (“carrier”) is inoculated with a representative test virus and the virus is dried onto the carrier. The carrier is inoculated with an aliquot of the use dilution of the test substance (liquid products), or to the amount of spray released under use conditions (spray products). The inoculated carrier is held for the requested exposure time at the requested exposure temperature. Following exposure, the contents of the carrier are neutralized and serial dilutions of the neutralized test substance are performed. The dilutions are then assayed for viral infectivity by an assay method specific for the test virus. Appropriate virus, test substance cytotoxicity, and neutralization controls are run concurrently.

### LIST OF REFERENCES CITED

Cozad, A., and R. D. Jones. 2003. Disinfection and the Prevention of Infectious Disease. Am. J. Infect. Control 31:243-254.
Aitken. C., and D. J. Jeffries. 2001. Nosocomial spread of viral disease. Clin. Microbiol. Rev. 14:528-546., Barker. J., D. Stevens, and S. F. Bloomfield. 2001
Barker, J., D. Stevens, and S. F. Bloomfield. 2001. Spread and prevention of some common viral infections in community facilities and domestic homes. J. Appl. Microbiol. 91:7-21.
England, B. L. 1982. Detection of viruses on fomites, p. 179-220. In C. P. Gerba and S. M. Goyal (ed.), Methods in environmental virology. Marcel Dekker, Inc., New York, N.Y.;
Haas, C. N., J. B. Rose, and C. P. Gerba. 1999, In Microbial agents and their transmission, p. 35-50. In C. N. Haas, J. B. Rose, and C. P. Gerba (ed.), Quantitative microbial risk assessment. J. Wiley and Sons, Inc., New York, N.Y.;
Reynolds, K. A., P. Watts, S. A. Boone, and C. P. Gerba. 2005. Occurrence of bacteria and biochemical biomarkers on public surfaces. Int. J. Environ. Health Res. 15:225-234;
Sattar, S. A. 2001. Survival of microorganisms on animate and inanimate surfaces and their disinfection, p. 195-205. In W. A. Rutala (cd.), Disinfection, sterilization and antisepsis: principles and practices in healthcare facilities. Association for Professionals in Infection Control and Epidemiology, Inc., Washington, D.C.)
Goldmann, D. A. 2000. Transmission of viral respiratory infections in the home. Pediatr. Infect. Dis. J. 19 (Suppl. 10):S97-S102.)
Standard Test Method for Determining the Activity of Incorporated Antimicrobial Agent(s) in Polymeric or Hydrophobic Materials. ASTM International, West Conshohocken, Pa., 2007, Developed by Subcommittee: E35.15, Book of Standards Volume: 11.05. DOI: 10.1520/E2180-07R 12, www.astm.org/
Aheam D G, Grace D T, Jennings M J, Borazjani R N, Boles K J, Rose L J, et al. Effects of hydrogel/silver coatings on in vitro adhesion to catheters of bacteria associated with urinary tract infections. Curr Microbiol. 2000; 41:120-125._{[DW2]}

What is claimed:

1. A biologically activated, stable polymer composite comprising a fine particulate polymer salt ionically associated with a biologically active ionic agent, the polymer salt dispersed within a thermoset, thermoplastic or other curable polymer or curable polymer-containing mixture to form a biologically activated, curable polymer composite, wherein the biologically active agent remains intact and biologically active in the composite during preparation and after hardening or curing of the thermoset or thermoplastic or other curable polymer to form a solid-cured composite material or coating.

2. The biologically activated, stable polymer composite of claim 1, wherein the biologically active ionic agent is an anti-microbial agent.

3. The biologically activated, stable polymer composite of claim 2, wherein the anti-microbial agent is an antibiotic agent, an antiviral agent, an antifungal agent, an oligodynamic metal, or an antiparasitic agent.

4. The biologically activated, stable polymer composite of claim 3, wherein the anti-microbial agent is an oligodynamic metal.

5. The biologically activated, stable polymer composite of claim 4, wherein the oligodynamic metal is silver, copper, zinc, iron, gallium or bismuth.

6. The biologically activated, stable polymer composite of claim 1, wherein the biologically active ionic agent is a chemotherapeutic agent.

7. The biologically activated, stable polymer composite of claim 1, wherein the biologically active ionic agent is an anti-inflammatory agent.

8. The biologically activated, stable polymer composite of claim 1, wherein the biologically active ionic agent is selected from acetylsalicylic acid-CO2-, dexamethasone sodium phosphate, fusidic acid (fusidate), and vitamin C (ascorbate).

9. The biologically activated, stable polymer composite of claim 1, wherein the biologically active ionic agent is cationic.

10. The biologically activated, stable polymer composite of claim 1, wherein the biologically active ionic agent is anionic.

11. The biologically activated, stable polymer composite of claim 1, wherein the thermoset, thermoplastic or other curable polymer or curable polymer-containing mixture one or more polymer precursors selected from the group consisting of polysiloxane, polyalkylene, polyamide, epoxy, polycarbonate, polyester, vinyl, acrylic, and polyurethane polymer precursors, and combinations thereof.

12. The biologically activated, stable polymer composite of claim 1, wherein the fine particulate polymer salt comprises from about 1 to 75% weight percent of the solid biologically activated polymer composite.

13. The biologically activated, stable polymer composite of claim 1, formed into a solid by curing.

14. The biologically activated, stable polymer composite of claim 13, wherein the fine particulate polymer salt is evenly distributed within at least a portion of the solid biologically activated polymer composite.

15. The biologically activated, stable polymer composite of claim 13, wherein a portion of the solid biologically activated polymer composite is free of the fine particulate polymer salt.

16. The biologically activated, stable polymer composite of claim 1, comprising a liquid or semi-solid paint or coating composite formed by combining the biologically activated fine particulate polymer salt with one or more polymer paint precursors capable of forming a paintable, curable paint, coating or laminate.

17. The biologically activated, stable polymer composite of claim 16, wherein the polymer paint precursors are selected from or more acrylic, latex, polyester, enamel, polysiloxane, polyalkylene, polyamide, polycarbonate, polyvinyl, polyurethane, polyvinylidinefluoride (PVDF), plastisol, polyvinylchroide, varnish, glaze, shellac, and epoxy, polymer precursors, and combinations thereof.

18. The biologically activated, stable polymer composite of claim 1, comprising a liquid or semi-solid antifouling paint or coating composite formed by combining a biologically activated fine particulate polymer salt containing divalent copper (CuII) or zinc (ZnII) as an ionic biologically active agent with one or more polymer paint precursors capable of forming a paintable, curable paint, coating or laminate.

19. The biologically activated, stable polymer composite of claim 18, wherein the particulate polymer salt containing divalent copper (CuII) or zinc (ZnII) as the ionic biologically active agent is stabilized at least to a point of curing within a metal amine Lewis base complex.

20. The biologically activated, stable polymer composite of claim 18, wherein the liquid or semi-solid antifouling paint or coating composite includes one or more polymer precursors selected from acrylic, latex, polyester, enamel, polysiloxane, polyalkylene, polyamide, polycarbonate, polyvinyl, polyurethane, polyvinylidinefluoride (PVDF), plastisol, polyvinylchroide, varnish, glaze, shellac, and epoxy, polymer precursors, and combinations thereof.

21. The biologically activated, stable polymer composite of claim 18, wherein the polymer paint precursors are provided in a paint or coating mixture comprising one or more paint additives selected from surfactants, buffering agents, and preservatives.

22. The biologically activated, stable polymer composite of claim 1, formed from a polymer lacquer comprising a solvent that is evaporated from the polymer lacquer during hardening of the thermoset or thermoplastic or photocuring polymer.

23. The biologically activated, stable polymer composite of claim 1, wherein the biologically active agent exhibits less than 20% chemical loss or decomposition during preparation and hardening of the thermoset or thermoplastic or photocuring polymer.

24. The biologically activated, stable polymer composite of claim 23, wherein the biologically active agent exhibits less than 10% chemical loss or decomposition during preparation and hardening of the thermoset or thermoplastic or photocuring polymer.

25. The biologically activated, stable polymer composite of claim 24, wherein the biologically active agent exhibits less than 5% chemical loss or decomposition during preparation and hardening of the thermoset or thermoplastic or photocuring polymer.

26. The biologically activated, stable polymer composite of claim 1, wherein the biologically active agent exhibits less than 10% chemical loss or decomposition during preparation and curing or hardening of the thermoset or thermoplastic or photocuring polymer at polymer curing temperatures ranging between 150-250° C.

27. The biologically activated, stable polymer composite of claim 1, wherein the biologically active agent exhibits less than 10% chemical loss or decomposition during preparation and hardening of the thermoset or thermoplastic or photocuring polymer at polymer curing temperatures above 200° C.

28. The biologically activated, stable polymer composite of claim 1, wherein the biologically active agent exhibits less than 20% chemical oxidation, hydrolysis, decomposition, or photodegradation over a stable shelf period of the hardened polymer composite of at least six months at room temperature.

29. The biologically activated, stable polymer composite of claim 28, wherein the biologically active agent exhibits less than 10% chemical oxidation, hydrolysis, decomposition, or photodegradation over a stable shelf period of the hardened polymer composite of at least six months at room temperature.

30. The biologically activated, stable polymer composite of claim 1, wherein the biologically active agent exhibits less than 20% chemical oxidation, hydrolysis, decomposition, or photodegradation over a stable shelf period of the hardened polymer composite of at least one year at room temperature.

31. The biologically activated, stable polymer composite of claim 30, wherein the biologically active agent exhibits less than 10% chemical oxidation, hydrolysis, decomposition, or photodegradation over a stable shelf period of the hardened polymer composite of at least one year at room temperature.

32. The biologically activated, stable polymer composite of claim 1, wherein the biologically active agent exhibits less than 20% chemical oxidation, hydrolysis, decomposition, or photodegradation after exposure of the hardened polymer composite to heat exceeding 200° C. for one hour.

33. The biologically activated, stable polymer composite of claim 32, wherein the biologically active agent exhibits less than 10% chemical oxidation, hydrolysis, decomposition, or photodegradation after exposure of the hardened polymer composite to heat exceeding 200° C. for one hour.

34. The biologically activated, stable polymer composite of claim 33, wherein the biologically active agent exhibits less than 5% chemical oxidation, hydrolysis, decomposition, or photodegradation after exposure of the hardened polymer composite to heat exceeding 200° C. for one hour.

35. The biologically activated, stable polymer composite of claim 1, wherein the biologically active agent exhibits less than 20% chemical oxidation, hydrolysis, decomposition, or photodegradation after exposure of the hardened polymer composite to heat exceeding 300° C. for one hour.

36. The biologically activated, stable polymer composite of claim 35, wherein the biologically active agent exhibits less than 10% chemical oxidation, hydrolysis, decomposition, or photodegradation after exposure of the hardened polymer composite to heat exceeding 300° C. for one hour.

37. The biologically activated, stable polymer composite of claim 36, wherein the biologically active agent exhibits less than 5% chemical oxidation, hydrolysis, decomposition, or photodegradation after exposure of the hardened polymer composite to heat exceeding 300° C. for one hour.

38. A process for preparing a fine particulate ion-exchange polymer salt material biologically activated by ionic association with a biologically active ionic agent comprising: providing a plurality of particles of a water-insoluble polysulfonated, polycarboxylated, polyaminated, or polyphosphorylated polymer salt, the particles having a porous construction wherein individual particles define a channel, void or pore space surrounded by a wall or partition of polymer salt material;
combining the particles of ion-exchange polymer salt material with a biologically active ionic agent in an aqueous medium to substitute the biologically active ionic agent by salt-exchange for a counter-ion initially associated with the ion-exchange polymer salt material, to yield a biologically activated porous ion-exchange polymer salt particle having the biologically active ionic agent ionically associated with the ion-exchange polymer salt material, whereby the biologically active ionic agent is rendered insoluble and will not freely dissociate from the biologically activated polymer salt material in deionized water;
drying the biologically activated porous ion-exchange polymer salt particles to remove water;
milling the biologically activated porous ion-exchange polymer salt particles by a high energy milling process in the presence of a non-solvent liquid added to occupy channel, void and pore spaces within the polymer salt particles, whereby the non-solvent liquid provides compression resistance against interior surfaces of the particle walls and partitions opposing pressure and mechanical forces imparted on exterior surfaces of the walls and partition during the high energy milling, said compression resistance enhancing efficiency and uniformity of particle size reduction during milling by facilitating structural failure of walls and partitions throughout the porous polymer salt particle in response to the pressure and mechanical forces, yielding the fine particulate biologically activated ion-exchange polymer salt material.

39. The process of claim 38, wherein after drying the biologically activated porous ion-exchange polymer salt particles to remove water the dried particles are placed in a sealable milling container defining a milling chamber, and are combined with high-energy grinding media and a volume of the non-solvent liquid sufficient within the chamber, the non-solvent liquid occupying open void, channel and pore spaces in the activated polymer salt particles, wherein the milling the chamber is sealed during milling to contain the polymer salt particles, media and non-solvent fluid therein, and wherein after the high energy milling process is complete the chamber is unsealed and the fine particulate biologically activated ion-exchange polymer salt material is separated from the grinding media and the non-solvent liquid.

40. The process of claim 38, wherein the biologically activated polymer salt particles have an average diameter before high energy milling of between about 50 to about 2,500 μm.

41. The process of claim 38, wherein the high energy milling of the biologically activated porous ion-exchange polymer salt material in the presence of the non-solvent liquid yields an average milled particle diameter of the fine particulate biologically activated ion-exchange polymer salt material of between about 10 nm to 100 μm.

42. The process of claim 41, wherein the average milled particle diameter of the fine particulate biologically activated ion-exchange polymer salt material is between about 30 nm to 50 μm.

43. The process of claim 42, wherein the average milled particle diameter of the fine particulate biologically activated ion-exchange polymer salt material is between about 100 nm to 10 μm.

44. The process of claim 43, wherein the average milled particle diameter of the fine particulate biologically activated ion-exchange polymer salt material is between about 400 nm to 600 nm.

45. The process of claim 38, wherein high energy milling of the biologically activated porous ion-exchange polymer salt material in the presence of the non-solvent liquid yields an average milled particle diameter of the fine particulate biologically activated ion-exchange polymer salt material that is substantially uniform, having a standard deviation from a median particle size of ±1-3 micron or lower.

46. The process of claim 45, wherein the fine particulate biologically activated ion-exchange polymer salt material is milled to a uniform size having a standard deviation from a median particle size of ±0.75 micron or lower.

47. The process of claim 46, wherein fine particulate biologically activated ion-exchange polymer salt material is milled to a highly uniform size having a standard deviation from a median particle size of ±0.25 micron or lower.

48. The process of claim 38, wherein the biologically active ionic agent is an anti-microbial agent.

49. The process of claim 48, wherein the anti-microbial agent is an antibiotic agent, an antiviral agent, an antifungal agent, an oligodynamic metal, or an antiparasitic agent.

50. The process of claim 49, wherein the anti-microbial agent is an oligodynamic metal.

51. The process of claim 50, wherein the oligodynamic metal is silver, copper, zinc, iron, gallium or bismuth.

52. The process of claim 38, wherein the biologically active ionic agent is a chemotherapeutic agent.

53. The process of claim 38, wherein the biologically active ionic agent is an anti-inflammatory agent.

54. The process of claim 38, wherein the biologically active ionic agent is an alkaloid.

55. The process of claim 38, wherein the biologically active ionic agent is an analgesic agent.

56. The process of claim 38, wherein the biologically active agent is selected from a cationic antibiotic or antiseptic.

57. The process of claim 56, wherein the cationic antibiotic is selected from a tetracycline or anthracycline.

58. The process of claim 57, wherein the cationic antibiotic is a tetracycline.

59. The process of claim 58, wherein the tetracycline is selected from tetracycline, doxycycline, minocycline, lymecycline, or apicycline, or combinations thereof.

60. The process of claim 56, wherein the cationic antiseptic comprises a quanidinium group.

61. The process of claim 60, wherein the cationic antiseptic is selected from chlorhexidine or polyhexamethylenebiguanide.

62. The process of claim 60, wherein the cationic antiseptic comprises a quaternary ammonium group.

63. The process of claim 62, wherein the cationic antiseptic is selected from chlorhexidine, benzalkonium, cetylpyridinium, or quarternary ammonium, and combinations thereof.

64. The process of claim 38, wherein the biologically active ionic agent is anionic.

65. The process of claim 64, wherein the anionic biologically active ionic is selected from acetylsalicylic acid-CO2-, dexamethasone sodium phosphate, fusidic acid (fusidate), and vitamin C (ascorbate).

66. The process of claim 65, wherein the anionic biologically active agent is acetylsalicylic acid (CO2-)

67. The process of claim 65, wherein the anionic biologically active agent is dexamethasone sodium phosphate

68. The process of claim 65, wherein the anionic biologically active agent is fusidic acid (fusidate).

69. The process of claim 65, wherein the anionic biologically active agent is vitamin C (ascorbate).

70. The process of claim 65, wherein the anionic biologically active agent is an antifungal agent, anti-inflammatory agent or an anti-oxidant agent.

71. The process of claim 65, wherein the anionic biologically active agent is a contraceptive agent.

72. The process of claim 38, wherein the ion-exchange polymer salt material comprises one or more styrene, acrylic, acrylate, sulfonate, carboxylate, phosphate, protonated amine, ammonium, and/or quaternary ammonium functional group(s).

73. The process of claim 38, wherein the ion-exchange polymer salt material comprises a cross-linked polymer resin.

74. The process of claim 73, wherein the cross-linked polymer resin comprises a styrene, acrylic, or acrylate resin.

75. The process of claim 38, wherein the non-solvent liquid is an alkane.

76. The process of claim 75, wherein the alkane is selected from heptane, octane or iso-octane.

77. The process of claim 38, wherein the high energy milling is conducted at a controllable temperature ranging between about 70° C. to about 95° C. to optimize size reduction and uniformity of the fine particulate biologically activated ion-exchange polymer salt material.

78. The process of claim 38, wherein the high energy milling is conducted at a controllable temperature ranging between about 80° C. to about 90° C. to optimize size reduction and uniformity of the fine particulate biologically activated polymer salt material.

79. The process of claim 39, wherein the grinding media comprises ceramic beads.

80. The process of claim 39, wherein the grinding media comprises zirconium beads.

81. The process of claim 39, wherein the grinding media comprises beads or other structural units having an average diameter of about 5 mm.

82. The process of claim 39, wherein the non-solvent liquid is separated from the fine particulate biologically activated ion-exchange polymer salt material by controlled evaporation.

83. The process of claim 39, wherein the grinding media is separated from the fine particulate biologically activated ion-exchange polymer salt material by mechanical separation.

84. The process of claim 83, wherein the mechanical separation is sieving.

85. The process of claim 39, wherein the sealable milling container has an internal surface or lining having a hardness substantially the same as a hardness of the grinding media.

86. The process of claim 85, wherein the internal surface or lining and grinding media each comprise a ceramic material.

87. The process of claim 38, wherein friction and other mechanical forces generated by the high energy milling process generates heat to maintain a temperature of the porous biologically activated ion-exchange polymer salt particles during milling between about 70° C. to about 95° C.

88. The process of claim 38, wherein the high energy milling process is conducted at least for a portion of the milling process at a temperature of between about 80° C. to about 90° C.

89. A composition comprising a fine particulate ion-exchange polymer salt material biologically activated by ionic association with a biologically active ionic agent prepared according to the process of claim 38.

90. A composition comprising a fine particulate ion-exchange polymer salt material biologically activated by ionic association with a biologically active ionic agent prepared according to the process of claim 39.

91. The composition of claim 90, wherein the fine particulate biologically activated ion-exchange polymer salt material has an average milled particle diameter of between about 100 nm to 10 μm.

92. The composition of claim 91, wherein the average milled particle diameter of the fine particulate biologically activated ion-exchange polymer salt material is approximately 500 nm or smaller.

93. The composition of claim 91, wherein an average milled particle diameter is substantially uniform, having a standard deviation from a median particle size of ±1-3 micron or lower.

94. The composition of claim 91, wherein an average milled particle diameter conforms to a uniform size having a standard deviation from a median particle size of ±0.75 micron or lower.

95. The composition of claim 91, wherein an average milled particle diameter is highly uniform, having a standard deviation from a median particle size of ±0.25 micron or lower.

96. The composition of claim 90, wherein the biologically active ionic agent is an anti-microbial agent.

97. The composition of claim 96, wherein the anti-microbial agent is selected from the group consisting of an antibiotic agent, an antiviral agent, an antifungal agent, an oligodynamic metal, an antiparasitic agent, and combinations thereof.

98. The composition of claim 97, wherein the anti-microbial agent is an oligodynamic metal.

99. The composition of claim 98, wherein the oligodynamic metal is silver, copper, zinc, iron, gallium or bismuth.

100. The composition of claim 90, wherein the biologically active ionic agent is a chemotherapeutic agent.

101. The composition of claim 90, wherein the biologically active ionic agent is an anti-inflammatory agent.

102. The composition of claim 90, wherein the biologically active ionic agent is cationic.

103. The composition of claim 102, wherein the cationic biologically active agent is selected from a cationic antibiotic or antiseptic.

104. The composition of claim 103, wherein the cationic antibiotic is selected from a tetracycline or anthracycline.

105. The composition of claim 104, wherein the cationic antibiotic is a tetracycline.

106. The composition of claim 105, wherein the tetracycline is selected from tetracycline, doxycycline, minocycline, lymecycline, or apicycline, or combinations thereof.

107. The composition of claim 103, wherein the cationic antiseptic comprises a quanidinium group.

108. The composition of claim 103, wherein the cationic antiseptic is selected from chlorhexidine or polyhexamethylenebiguanide.

109. The composition of claim 103, wherein the cationic antiseptic comprises a quaternary ammonium group.

110. The composition of claim 103, wherein the cationic antiseptic is selected from chlorhexidine, benzalkonium, cetylpyridinium, or quarternary ammonium, and combinations thereof.

111. The composition of claim 90, wherein the biologically active ionic agent is anionic.

112. The composition of claim 111, wherein the anionic biologically active ionic is selected from acetylsalicylic acid-CO2-, dexamethasone sodium phosphate, fusidic acid (fusidate), and vitamin C (ascorbate).

113. The process of claim 112, wherein the anionic biologically active agent is acetylsalicylic acid (CO2-)

114. The process of claim 112, wherein the anionic biologically active agent is dexamethasone sodium phosphate

115. The process of claim 112, wherein the anionic biologically active agent is fusidic acid (fusidate).

116. The process of claim 112, wherein the anionic biologically active agent is vitamin C (ascorbate).

117. The composition of claim 90, wherein the ion-exchange polymer salt material comprises one or more of styrene, acrylic, acrylate, sulfonate, carboxylate, phosphate, protonated amine, ammonium, and/or quaternary ammonium.

118. The composition of claim 90, wherein the ion-exchange polymer salt material comprises a cross-linked polymer resin.

119. The composition of claim 118, wherein the cross-linked polymer resin comprises a styrene, acrylic, or acrylate resin.

120. The process of claim 39, wherein the high energy milling is a multi-stage process involving multiple cycles of milling and multiple grades of grinding media, wherein after a first milling cycle a partially-milled biologically activated ion-exchange polymer salt material is separated from a first-cycle grinding media and non-solvent liquid and then combined in the sealable milling container with a second-cycle, smaller grinding media and a second volume of non-solvent liquid, followed by a second cycle of milling to generate a desired particle size and uniformity of the fine particulate biologically activated ion-exchange polymer salt material.

121. The process of claim 120, wherein the same non-solvent liquid is used in both the first and second milling cycles.

122. The process of claim 120, wherein ceramic grinding media are used in both the first and second milling cycles.

123. The process of claim 120, wherein the second cycle grinding media are between approximately 0.5 to 5.0 mm in diameter.

124. The process of claim 120, wherein the second cycle grinding media are approximately 1 mm in diameter.

125. The process of claim 120, wherein after the second milling cycle the non-solvent liquid is separated from the fine particulate biologically activated ion-exchange polymer salt material by evaporation.

126. The process of claim 120, wherein after the second milling cycle the grinding media is separated from the fine particulate biologically activated ion-exchange polymer salt material by mechanical separation.

127. The process of claim 120, wherein the fine particulate biologically activated ion-exchange polymer salt material is milled to a size between about 100 nm to about 10 μm.

128. The process of claim 120, wherein the fine particulate biologically activated ion-exchange polymer salt material is milled to a size between about 400 nm to about 600 nm.

129. The process of claim 120, wherein the fine particulate biologically activated ion-exchange polymer salt material is milled to a substantially uniform size, having a standard deviation from a median particle size of ±1-3 micron or lower.

130. The process of claim 120, wherein the fine particulate biologically activated ion-exchange polymer salt material is milled to a highly uniform size, having a standard deviation from a median particle size of ±0.25 micron or lower.

131. A process for preparing a biologically activated polymer composite comprising: providing a plurality of particles comprising a water-insoluble polysulfonated, polycarboxylated, polyaminated, or polyphosphorylated polymer salt, the particles having a porous construction wherein individual particles define a channel, void or pore space surrounded by a wall or partition of polymer salt material;
combining the particles of ion-exchange polymer salt material with a biologically active ionic agent in an aqueous medium to substitute the biologically active ionic agent by salt-exchange for a counter-ion initially associated with the ion-exchange polymer salt material, to yield a biologically activated porous ion-exchange polymer salt particle having the biologically active ionic agent ionically associated with the ion-exchange polymer salt material, whereby the biologically active ionic agent is rendered insoluble and will not freely dissociate from the ion-exchange polymer salt material in deionized water;
drying the biologically activated porous ion-exchange polymer salt particles to remove water;
milling the biologically activated porous ion-exchange polymer salt particles by a high energy milling process in the presence of a non-solvent liquid added to occupy channel, void and pore spaces within the polymer salt particles, whereby the non-solvent liquid provides compression resistance against interior surfaces of the particle walls and partitions opposing pressure and mechanical forces imparted on exterior surfaces of the walls and partition during the high energy milling, said compression resistance enhancing efficiency and uniformity of particle size reduction during milling by facilitating structural failure of walls and partitions throughout the porous polymer salt particle in response to the pressure and mechanical forces, yielding the fine particulate biologically activated ion-exchange polymer salt material;
blending the fine particulate biologically activated ion-exchange polymer salt material with one or more thermoset, thermoplastic, photocuring or other curable polymer precursors to form a fluid or semi-solid thermoset or thermoplastic or photocuring polymer and biologically activated ion-exchange polymer salt composite mixture.

132. The process for preparing a biologically activated polymer composite of claim 131, further comprising solidifying the thermoset or thermoplastic or photocuring polymer precursors to form a biologically activated solidified polymer composite comprising the fine particulate biologically activated modified polymer salt dispersed within the thermoset or thermoplastic or photocuring or other curable polymer to form a solid biologically activated polymer composite.

133. The method of claim 131, wherein the thermoset or thermoplastic, photocuring or other curable polymer is selected from the group consisting of polysiloxane, polyalkylene, polyamide, epoxy, polycarbonate, polyester, vinyl, acrylic, polyurethane, polymers and combinations thereof.

134. The method of claim 131, wherein the polymer precursors comprise nonvulcanized silicone rubber precursors.

135. The method of claim 134, wherein the silicone rubber precursors combine to form a highly-adhesive silicone gel or liquid.

136. The method of claim 131, wherein the polymer precursors comprise silicone gel or liquid curable to yield a hardened silicone product.

137. The method of claim 136, further comprising curing the silicone gel, rubber or liquid to a solid form at about 150° C. for a curing period of between about 5 to 10 minutes.

138. The method of claim 137, wherein the biologically active ionic agent is an oligodynamic metal.

139. The method of claim 138, wherein initial curing of the silicone gel, rubber or liquid further comprising an oligodynamic metal darkens the hardened silicone product.

140. The method of claim 138, wherein the hardened silicone product is further cured for an extended curing period at an optionally elevated curing temperature of between about 150-200° C.

141. The method of claim 140, wherein the hardened silicone product lightens after extended curing.

142. The method of claim 138, wherein the silicon polymer composite is post-cured beyond a conventional curing time and/or temperature to form a post-cured composite having improved, lightened color properties for medical use.

143. The method of claim 132, wherein the biologically activated solid polymer composite exhibits desired biological activity using a composite mixture incorporating as little 1-5% of the fine particulate biologically activated ion-exchange polymer salt material by weight.

144. The method of claim 132, wherein the biologically activated solid polymer composite incorporates between 5-10% of the fine particulate biologically activated ion-exchange polymer salt material by weight.

145. The method of claim 132, wherein the biologically activated solid polymer composite incorporates between 10-25% of the fine particulate biologically activated ion-exchange polymer salt material by weight.

146. The method of claim 132, wherein the biologically activated solid polymer composite incorporates between 25-45% of the fine particulate biologically activated ion-exchange polymer salt material by weight.

147. The method of claim 132, wherein the biologically activated solid polymer composite incorporates between 45-75% of the fine particulate biologically activated ion-exchange polymer salt material by weight.

148. The method of claim 132, wherein the fine particulate biologically activated ion-exchange polymer salt material is evenly distributed within at least a portion of the biologically activated solid polymer composite.

149. The method of claim 132, wherein a portion of the biologically activated solid polymer composite is free of the fine particulate biologically activated ion-exchange polymer salt material.

150. The method of claim 132, further comprising casting, molding, extruding, layering, laminating or painting the fluid or semi-solid polymer and biologically activated ion-exchange polymer salt composite mixture prior to solidifying the thermoset or thermoplastic or photocuring polymer.

151. The method of claim 132, wherein the thermoset, thermoplastic, photocuring or other curable polymer precursors are provided in the form of a polymer lacquer, the lacquer comprising a solvent, the solidifying step comprising evaporating the solvent from the polymer lacquer to form the solid biologically active polymer composite.

152. The method of claim 132, wherein the solidifying step comprises cooling a thermoplastic polymer mixture from a substantially fluid state.

153. A method of regenerating the surface of a biologically stable composite comprising a fine particulate ion-exchange polymer salt ionically associated with an ionic biologically active agent, admixed in a fine particulate form with a secondary polymer and hardened to form a composite, comprising abrading the surface to remove an exhausted outer portion of the composite material wherein the biologically active agent is integrated within a hardened surface of the composite.

154. A method of regenerating surface biological activity of a biologically activated polymer composite comprising a polymer salt ionically associated with a biologically active ionic agent admixed in a fine particulate form with a secondary polymer and thermoset or hardened to form a biologically activated composite material, comprising abrading the surface to remove an exhausted outer portion of the composite material to newly expose biologically active agent at a polished surface of the composite material.

155. The method of claim 153, wherein the abraded surface is polished such that there are no contaminable pores or voids.

156. The method of claim 153, wherein the surface is abraded with abrasive sheets, pastes, and gels.

157. A method of regenerating surface biological activity of a biologically activated polymer composite material comprising a fine particulate polymer salt ionically associated with a biologically active ionic agent admixed in a fine particulate form with a secondary polymer and thermoset or hardened to form a biologically activated composite material, comprising exposing the surface to an ion-exchange liquid that mediates ion-exchange addition of new biologically active ionic to recharge initial polymer salt associations at a partially or fully exhausted surface of the composite material.

158. The method of claim 157, wherein the ion-exchange liquid comprises silver acetate, copper chloride or copper salt.

159. The method of claim 157, wherein exposure of the surface of the partially or fully exhausted composite material to the ion-exchange liquid restores at least 10-50% of initial surface activity of the composite material.

160. A method of activating surface biological activity of a polymer composite comprising a fine particulate polymer salt ionically associated with a biologically active ionic agent admixed in a fine particulate form with a secondary polymer and thermoset or hardened to form a biologically activated composite material, comprising exposing the surface to peroxide.

161. The method of claim 160, wherein exposing the surface to peroxide generates superoxides at the surface.

162. A method of recharging surface biological activity of a polymer composite comprising a fine particulate polymer salt ionically associated with a biologically active ionic agent admixed in a fine particulate form with a secondary polymer and thermoset or hardened to form a biologically activated composite material, comprising exposing the surface to peroxide.

163. The method of claim 162, wherein exposing the surface to peroxide generates superoxides at the surface.
